(19) United States
(12) Patent Application Publication
(10) Pub. No.: US 2012/0184031 A1
(43) Pub. Date: Jul. 19, 2012

(54) ANTISENSE INHIBITION VIA RNASE H-INDEPENDENT REDUCTION IN MRNA

(75) Inventors: Brett P. Monia, Encinitas, CA (US); Susan M. Freier, San Diego, CA (US); Muthiah Manoharan, Weston, MA (US); William A. Gaarde, Carlsbad, CA (US); Richard H. Griffey, Vista, CA (US); Eric E.` Swayze, Carlsbad, CA (US); C. Frank Bennett, Carlsbad, CA (US)

(73) Assignee: Isis Pharmaceuticals, Inc., Carlsbad, CA (US)

(21) Appl. No.: 13/351,511

(22) Filed: Jan. 17, 2012

Related U.S. Application Data
(63) Continuation of application No. 10/948,947, filed on Sep. 24, 2004, now abandoned.
Application No. 10/948,947 is a continuation-in-part of application No. 10/461,163, filed on Jun. 13, 2003, now abandoned.
(60) Provisional application No. 60/392,020, filed on Jun. 26, 2002.

(51) Int. Cl. C12N 5/071

(52) U.S. Cl. 435/366

(57) ABSTRACT

The present invention provides compositions and methods for reducing levels of a preselected mRNA, using antisense compounds targeted to a splice site or a region up to 50 nucleobases upstream of an exon/intron junction on said mRNA. Preferably, said antisense compounds do not elicit RNAse H cleavage of the mRNA.

This application is a continuation of U.S. application Ser. No. 10/948,947, filed Sep. 24, 2004, which is a continuation-in-part of U.S. application Ser. No. 10/461,163, filed Jun. 13, 2003, which claims the benefit of priority to U.S. provisional application Ser. No. 60/392,020, filed Jun. 26, 2002.

### SEQUENCE LISTING

The present application is being filed along with a Sequence Listing in electronic format. The Sequence Listing is provided as a file entitled ISPH0871USC1SEQ.txt, created on Jan. 16, 2012, which is 211 Kb in size. The information in the electronic format of the sequence listing is incorporated herein by reference in its entirety.

### FIELD OF THE INVENTION

The present invention provides compositions and methods for reducing gene expression. In particular, antisense compositions and methods are provided for reducing RNA levels via mechanisms that are believed to be RNAse H-independent. The antisense compounds may be targeted to a splice site or a region up to 50 nucleobases 5′ of an exon/intron junction on the target mRNA.

### BACKGROUND OF THE INVENTION

Newly synthesized eukaryotic mRNA molecules, also known as primary transcripts or pre-mRNA, made in the nucleus, are processed before or during transport to the cytoplasm for translation. A methylated cap structure, consisting of a terminal nucleotide, 7-methylguanylate, is added to the 5′-end of the mRNA in a 5′-5′ linkage with the first nucleotide of the mRNA sequence. An approximately 200-250-base sequence of adenylate residues, referred to as poly(A), is added posttranscriptionally to a site that will become the 3′ terminus of the mRNA, before entry of the mRNA into the cytoplasm. This is a multistep process which involves assembly of a processing complex, then site-specific endonucleolytic cleavage of the precursor transcript, and addition of a poly(A) “tail.” In most mRNAs the polyadenylation signal sequence is a hexamer, AAUAAA, located 10 to 30 nucleotides in the 5′ direction (upstream) from the site of cleavage (5′-CA-3′) in combination with a U or G-U rich element 3′ to the cleavage site. Multiple poly(A) sites may be present on a given transcript, of which only one is used per transcript, but more than one species of mature mRNA transcript can be produced from a given pre-mRNA via use of different poly(A) sites. It has recently been shown that stable mRNA secondary structure can affect the site of polyadenylation of an RNA construct in transfected cells. Klasens et al., Nuc. Acids Res., 1998, 26, 1870-1876. It has also been found that which of multiple polyadenylation sites is used can affect transcript stability. Chu et al., J. Immunol., 1994, 153, 4179-4189.

The next step in mRNA processing is splicing of the mRNA, which occurs in the maturation of 90-95% of mammalian mRNAs. Introns (or intervening sequences) are regions of a primary transcript (or the DNA encoding it) that are not included in the coding sequence of the mature mRNA. Exons are regions of a primary transcript that remain in the mature mRNA when it reaches the cytoplasm. The exons are “spliced” together to form the mature mRNA sequence. Splice junctions are also referred to as “splice sites” with the 5′ side of the junction often called the “5′ splice site,” or “splice donor site” and the 3′ side the “3′ splice site” or “splice acceptor site.” In splicing, the 3′ end of an upstream exon is joined to the 5′ end of the downstream exon. Thus the unspliced RNA (or pre-mRNA) has an exon/intron junction at the 5′ end of an intron and an intron/exon junction at the 3′ end of an intron; after the intron is removed the exons are contiguous at what is sometimes referred to as the exon/exon junction or boundary in the mature mRNA. “Cryptic” splice sites are those which are less often used but may be used when the usual splice site is blocked or unavailable. Alternative splicing, i.e., the splicing together of various combinations of exons, often results in multiple mRNA transcripts from a single gene.

A final step in RNA processing is turnover or degradation of the mRNA. Differential mRNA stabilization is one of several factors in the rate of synthesis of any protein. mRNA degradation rates seem to be related to presence or absence of poly(A) tails and also to the presence of certain sequences in the 3′ end of the mRNA.

For example, many mRNAs with short half-lives contain several A(U)ₙA sequences in their 3′-untranslated regions. When a series of AUUUA sequences was inserted into a gene not normally containing them, the half life of the resulting mRNA decreased by 80%. Shaw and Kamen, Cell, 1986, 46, 659. This may be related to an increase of nucleolytic attack in sequences containing these A(U)ₙA sequences. Other mediators of mRNA stability are also known, such as hormones, translation products (autoregulation/feedback), and low-molecular weight ligands.

Degradation of mRNA can also occur through nonsense-mediated decay. After splicing of an mRNA, exon junction complexes, which are comprised of numerous different proteins, are formed 20-24 nucleotides upstream of exon/exon junctions. It is thought that exon junction complexes contribute to mRNA export to the cytoplasm. Ishigaki et al., 2001, Cell, 19, 6860-6869. As translation proceeds, the ribosome displaces any exon junction complexes in its path. If any exon junction complexes remain after a first round (also referred to as the “pioneer” round) of translation, the mRNA is a target for nonsense-mediated decay. The pioneer round of translation is complete when the ribosome reaches a stop codon, which triggers release factors to interact with any undisplaced exon junction complexes, leading to decapping of the transcript and subsequent mRNA degradation. Typically, mRNA transcripts with termination codons more than about 50 nucleotides 5′ of the final exon have undisplaced complexes, thus rendering the mRNAs targets for nonsense-mediated decay. Lewis et al., 2003, Proc. Natl. Acad. Sci. U.S.A., 100, 189-192.

Antisense compounds have generally been used to interfere with protein expression, either by interfering directly with translation of the target molecule or, more often, by RNAse H-mediated degradation of the target mRNA. Antisense interference with 5′ capping of mRNA and prevention of translation factor binding to the mRNA by oligonucleotide masking of the 5′ cap have been disclosed by Baker et al. (WO 91/17755). Antisense oligonucleotides have been used to modulate or redirect splicing, particularly aberrant splicing or splicing of mutant transcripts, often in cell-free reporter systems. A luciferase reporter plasmid system has been used to test the ability of antisense oligonucleotides targeted to the 5′ splice site, 3′ splice site or branchpoint to inhibit splicing of mutated or wild-type adenovirus pre-mRNA sequences in a luciferase reporter plasmid. Treatment with uniform 2′-O-methyl oligonucleotides caused an increase in luciferase mRNA and concomitant decrease in luciferase pre-mRNA in adenovirus constructs. In other words, target gene expression was increased by antisense treatment. However, when the constructs also contained human β-globin splice site sequences, the luciferase pre-mRNA was increased and the luciferase mRNA was decreased. The authors conclude that antisense oligonucleotides that can support RNAse H cleavage of target mRNA are the best inhibitors of efficiently processed pre-mRNA but that modified oligonucleotides that work by occupancy rather than RNA cleavage may be useful for less efficiently spliced targets. Hodges and Crooke, Mol. Pharmacol., 1995, 48, 905-918.

Kulka et al. reported use of a methylphosphonate antisense oligonucleoside complementary to the acceptor splice junction of herpes simplex virus type 1 immediate early mRNA 4 (1E4) to inhibit growth of this virus. The antisense oligonucleotide, which is believed not to be a substrate for RNAse H, inhibited viral protein synthesis. A 20% reduction in the amount of spliced 1E4 viral mRNA was accompanied by an equivalent increase in the amount of unspliced mRNA. Proc. Natl. Acad. Sci. (USA), 1989, 86, 6868-6872.

Antisense oligonucleotides have been used to target mutations that lead to aberrant splicing in several genetic diseases, in order to redirect splicing to give a desired splice product. Phosphorothioate 2′-O-methyl oligoribonucleotides have been used to target the aberrant 5′ splice site of the mutant β-globin gene found in patients with β-thalassemia, a genetic blood disorder. Aberrant splicing of mutant β-globin mRNA was blocked and normal splicing was restored in vitro in vector constructs containing thalassemic human β-globin pre-mRNAs using 2′-O-methyl-ribo-oligonucleotides targeted to the branch point sequence in the first intron of the mutant human β-globin pre mRNAs. 2′-O-methyl oligonucleotides are used because they are stable to RNAses and form stable hybrids with RNA that are not degraded by RNAse H. Dominski and Kole, Proc. Natl. Acad. Sci. USA, 1993, 90, 8673-8677. A review article by Kole discusses use of antisense oligonucleotides targeted to aberrant splice sites created by genetic mutations such as β-thalassemia or cystic fibrosis. It was hypothesized that blocking a splice site with an antisense oligonucleotide will have similar effect to mutation of the splice site, i.e., redirection of splicing. Kole, Acta Biochimica Polonica, 1997, 44, 231-238. Oligonucleotides targeted to the aberrant β-globin splice site suppressed aberrant splicing and at least partially restored correct splicing in HeLa cells expressing the mutant transcript. Sierakowska et al., Nucleosides & Nucleotides, 1997, 16, 1173-1182; Sierakowska et al., Proc. Natl. Acad. Sci. USA, 1996, 93, 12840-44. U.S. Pat. No. 5,627,274 discloses and WO 94/26887 discloses and claims compositions and methods for combating aberrant splicing in a pre-mRNA molecule containing a mutation, using antisense oligonucleotides which do not activate RNAse H.

Modulation of mutant dystrophin splicing with 2′-O-methyl oligoribonucleotides has been reported both in vitro and in vivo. In dystrophin Kobe, a 52-base pair deletion mutation causes exon 19 to be skipped during splicing. An in vitro minigene splicing system was used to show that a 31-mer 2′-O-methyl oligoribonucleotide complementary to the 5′ half of the deleted sequence in dystrophin Kobe exon 19 inhibited splicing of wild-type pre-mRNA. Takeshima et al., J. Clin. Invest., 1995, 95, 515-520. The same oligonucleotide was used to induce exon skipping from the native dystrophin gene transcript in human cultured lymphoblastoid cells.

Dunckley et al., (Nucleosides & Nucleotides, 1997, 16, 1665-1668) describes in vitro constructs for analysis of splicing around exon 23 of mutated dystrophin in the mdx mouse mutant, a model for Duchenne muscular dystrophy. Plans to analyze these constructs in vitro using 2′ modified oligos targeted to splice sites within and adjacent to mouse dystrophin exon 23 are discussed, though no target sites or sequences are given. 2′-O-methyl oligoribonucleotides were subsequently used to correct dystrophin deficiency in myoblasts from the mdx mouse. An antisense oligonucleotide targeted to the 3′ splice site of murine dystrophin intron 22 caused skipping of the mutant exon and created a novel in-frame dystrophin transcript with a novel internal deletion. This mutated dystrophin was expressed in 1-2% of antisense treated mdx myotubes. Use of other oligonucleotide modifications such as 2′-O-methoxyethyl phosphodiesters are disclosed. Dunckley et al. (Human Mol. Genetics, 1998, 5, 1083-90).

Phosphorothioate oligodeoxynucleotides have been used to selectively suppress the expression of a mutant α2 (I) collagen allele in fibroblasts from a patient with osteogenesis imperfecta, in which a point mutation in the splice donor site produces mRNA with exon 16 deleted. The oligonucleotides were targeted either to the point mutation in the pre-mRNA or to the defectively spliced transcript. In both cases mutant mRNA was decreased by half but the normal transcript is also decreased by 20%. This was concluded to be fully accounted for by an RNAse H-dependent mechanism. Wang and Marini, J. Clin Invest., 1996, 97, 448-454.

A microinjection assay was used to test the antisense effects on SV40 large T antigen (TAg) expression of oligonucleotides containing C-5 propynylpyrimidines, either as 2′-O-allyl phosphodiester oligonucleotides, which do not elicit RNAse H cleavage of the target, or as 2′-deoxy phosphorothioates, which do elicit RNAse H cleavage. Oligonucleotides targeted to the 5′ untranslated region, translation initiation site, 5′ splice junction or polyadenylation signal of the TAg transcript were injected into the nucleus or cytoplasm of cultured cells. The only 2′-O-allyl (non-RNAse H) oligonucleotides which were effective at inhibiting T-antigen were those targeted to the 5′ untranslated region and the 5′ splice junction. The 2′-O-allyl phosphodiester/C-5 propynylpyrimidine oligonucleotides, which do not elicit RNAse H, were 20 fold less potent than the oligodeoxynucleotides which had the ability to recruit RNAse H. The authors concluded that the duplexes formed between the RNA target and the 2′-O-allyl phosphodiester/C-5 propynylpyrimidine oligonucleotides dissociate rapidly in cells. Moulds et al., 1995, Biochem., 34, 5044-53. Biotinylated 2′-O-allyloligoribonucleotides incorporating 2-aminoadenine bases were targeted to the U2 small nuclear RNA (snRNA), a component of the spliceosome, in HeLa nuclear extracts. These inhibited mRNA production with a concomitant accumulation of splicing intermediates. Barabino et al., Nucl. Acids Res., 1992, 20, 4457-4464.

Thus antisense oligonucleotides are used in the art to redirect splicing or to prevent splicing. In neither mechanism is there a net loss of target mRNA in cells (though one splice product may decrease in proportion to the accumulation of another splice product or products, or of unspliced RNA). Generally, oligonucleotides which are not substrates for RNAse H are preferred where redirection of splicing is desired, as the goal is production of a desired mRNA rather than a loss of mRNA as would be expected through use of an oligonucleotide which, when duplexed with RNA, is a substrate for RNAse H cleavage of the RNA.

There is, therefore a continued need for additional compositions and methods for reducing target mRNA levels, thus reducing expression of the corresponding protein product. The present invention provides antisense compounds and methods for such modulation. The compositions and methods of the invention can be used in therapeutics, including prophylaxis, and as research tools.

It has now been found that targeting antisense compounds to a splice site or a region up to 50 nucleobases 5′ of an exon/intron junction of a target mRNA can result in loss or partial loss of the target RNA, even though the antisense compounds are modified in such a way that they are not substrates for RNAse H. While not wishing to be bound by theory, it is believed that such decrease in target RNA is a result of RNA degradation or cleavage, presumably via a non-RNAse H mechanism. Accordingly, antisense compounds which do not elicit RNAse H cleavage are preferred for use in the invention.

### SUMMARY OF THE INVENTION

The present invention provides methods for reducing amounts of a selected wild-type mRNA target within a cell, by binding to the mRNA target an antisense compound which is specifically hybridizable to a region up to 50 nucleobases 5′ of an exon/intron junction on the mRNA target and which preferably does not support RNAse H cleavage of the mRNA target upon binding. It has now been found that in spite of not being a substrate for RNAse H, antisense compounds targeted to the region upstream of exon/intron junctions can cause a decrease in target mRNA levels.

In one aspect of the invention, the antisense compound is an antisense oligonucleotide. Preferably, the antisense compound is targeted to at least a portion of a region up to 50 nucleobases upstream of an exon/intron junction of a target mRNA. More preferably the antisense compound is targeted to at least a portion of a region 20-24 or 30-50 nucleobases upstream of an exon/intron junction. Preferably, the antisense compound contains at least one modification which increases binding affinity for the mRNA target and which increases nuclease resistance of the antisense compound. In one aspect, the antisense compound comprises at least one nucleoside having a 2′ modification of its sugar moiety. Advantageously, every nucleoside of the antisense compound has a 2′ modification of its sugar moiety. Preferably, the 2′ modification is 2′-fluoro or 2′-methoxyethyl (2′-MOE). In another aspect of this preferred embodiment, the antisense compound contains at least one modified backbone linkage other than a phosphorothioate backbone linkage. The antisense compound may also comprise one or more modified backbone linkages other than phosphorothioate backbone linkages. Preferably, the antisense compound also comprises at least one phosphodiester or phosphorothioate backbone linkage. In one aspect of the invention, the modified backbone linkages alternate with phosphodiester and/or phosphorothioate backbone linkages. Advantageously, substantially every backbone linkage is a modified backbone linkage other than a phosphorothioate linkage. Preferably, the modified backbone linkage may be a 3′-methylene phosphonate, locked nucleic acid (LNA), peptide nucleic acid (PNA) or morpholino linkage. In one aspect of this preferred embodiment, the modified backbone linkage is a peptide nucleic acid, wherein said peptide nucleic acid has a cationic tail bound thereto. Preferably, the cationic tail comprises one or more, preferably one to four, lysine or arginine residues. In another aspect of this embodiment, the peptide nucleic acid is conjugated to a protein that binds to exon junction complexes. In addition, the antisense compound may contain at least one modified nucleobase. Preferably, the modified nucleobase is a C-5 propyne or 5-methyl C.

### DETAILED DESCRIPTION OF THE INVENTION

The present invention employs oligomeric antisense compounds, particularly oligonucleotides, for decreasing the levels of a preselected target mRNA, ultimately decreasing the expression of the protein encoded by said target mRNA.

Modulation of mRNA levels is achieved by targeting a splice site or a region up to 50 nucleobases 5′ of an exon/intron junction on the target mRNA with antisense oligonucleotides. Surprisingly, it has now been found that it is not necessary that the oligonucleotides elicit RNAse H cleavage of the target RNA in order to reduce RNA levels. While not wishing to be bound by theory, it is presently believed that inhibition of either normal splicing or pioneer translation may result in degradation of the improperly processed RNA. Thus it is preferred that the oligonucleotides of the invention do not elicit RNAse H cleavage of the target RNA strand. Preferably, the RNA to be targeted is a cellular mRNA and the antisense compound is contacted with said cellular mRNA within a cell.

Data from a variety of molecular targets are provided as illustrations of the invention. As used herein, the terms “target nucleic acid” and “nucleic acid encoding a target” encompass DNA encoding a given molecular target (i.e., a protein or polypeptide), RNA (including pre-mRNA and mRNA) transcribed from such DNA, and also cDNA derived from such RNA. The specific hybridization of an antisense compound with its target nucleic acid interferes with the normal function of the nucleic acid. This modulation of function of a target nucleic acid by compounds which specifically hybridize to it is generally referred to as “antisense”. The functions of DNA to be interfered with include replication, transcription and translation. The overall effect of such interference with target nucleic acid function is modulation of the expression of the target molecule. In the context of the present invention, “modulation” means a quantitative change, either an increase (stimulation) or a decrease (inhibition), for example in the expression of a gene. Inhibition of gene expression through reduction in RNA levels is a preferred form of modulation according to the present invention.

It is preferred to target specific nucleic acids for antisense. “Targeting” an antisense compound to a particular nucleic acid, in the context of this invention, is a multistep process. The process usually begins with the identification of a nucleic acid sequence whose expression is to be modulated. This may be, for example, a cellular gene (or mRNA transcribed from the gene) whose expression is associated with a particular disorder or disease state, or a nucleic acid molecule from an infectious agent. The targeting process also includes determination of a site or sites within this gene for the antisense interaction to occur such that the desired effect, e.g., reduction of RNA levels, will result. In the context of the present invention, splice sites, particularly intron/exon and exon/intron junctions, and regions up to 50 nucleobases upstream of exon/intron junctions, are preferred target sites. Once one or more target sites have been identified, oligonucleotides are chosen which are sufficiently complementary to the target, i.e., hybridize sufficiently well and with sufficient specificity, to give the desired effect. In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds.

“Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

Antisense compounds are commonly used as research reagents and diagnostics. For example, antisense oligonucleotides, which are able to inhibit gene expression with exquisite specificity, are often used by those of ordinary skill to elucidate the function of particular genes. Antisense compounds are also used, for example, to distinguish between functions of various members of a biological pathway. Antisense modulation has, therefore, been harnessed for research use.

The specificity and sensitivity of antisense is also harnessed by those of skill in the art for therapeutic uses. Antisense oligonucleotides have been employed as therapeutic moieties in the treatment of disease states in animals and man. Antisense oligonucleotides have been safely and effectively administered to humans and numerous clinical trials are presently underway. An antisense oligonucleotide drug, Vitravene™, has been approved by the U.S. Food and Drug Administration for the treatment of cytomegalovirus retinitis (CMVR), a cause of blindness, in AIDS patients. It is thus established that oligonucleotides can be useful therapeutic modalities that can be configured to be useful in treatment regimes for treatment of cells, tissues and animals, especially humans.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 80 nucleobases (i.e. from about 8 to about 80 linked nucleosides). Particularly preferred antisense compounds are antisense oligonucleotides, even more preferably those comprising from about 10 to about 50 nucleobases, more preferably from about 13 to about 30 nucleobases. Antisense compounds include ribozymes, external guide sequence (EGS) oligonucleotides (oligozymes), and other short catalytic RNAs or catalytic oligonucleotides which hybridize to the target nucleic acid and modulate its expression.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. In addition, linear structures may also have internal nucleobase complementarity and may therefore fold in a manner as to produce a double stranded structure. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thiono-alkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Most preferred embodiments of the invention are oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506, the contents of which are incorporated herein in their entirety.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylamino-ethoxyethoxy (also known in the art as 2′-O-dimethylamino-ethoxyethyl or 2′-DMAEOE), i.e., 2′-O—CH₂—O—CH₂—N(CH₂)₂, also described in examples hereinbelow.

Other preferred modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

A further preferred modification includes Locked Nucleic Acids (LNAs) in which the 2′-hydroxyl group is linked to the 3′ or 4′ carbon atom of the sugar ring thereby forming a bicyclic sugar moiety. The linkage is preferably a methylene (—CH₂—)ₙ group bridging the 2′ oxygen atom and the 4′ carbon atom wherein n is 1 or 2. LNAs and preparation thereof are described in WO 98/39352 and WO 99/14226. ENAs, similar to LNAs except that the sugar ring is a hexenyl instead of a furanose, as described in WO 01/49687 are also included, as are other heterocyclic bicyclic nucleic acids.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, propynes, e.g., 5-propynyl (—C≡C—CH₃) uracil and cytosine and other alkynyl derivatives of pyrimidine bases disclosed in U.S. Pat. No. 6,235,887, the contents of which are incorporated by reference herein; 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one), or guanidinium G-clamps and analogs. Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyl-adenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and'Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,681,941; 6,028,183 and 6,007,992, certain of which are commonly owned with the instant application, and each of which is herein incorporated by reference, and U.S. Pat. No. 5,750,692, which is commonly owned with the instant application and also herein incorporated by reference.

Another modification of the oligonucleotides of the invention involves chemically linking to the oligonucleotide one or more moieties or conjugates which enhance the activity, cellular distribution or cellular uptake of the oligonucleotide. The compounds of the invention can include conjugate groups covalently bound to functional groups such as primary or secondary hydroxyl groups. Conjugate groups of the invention include inter-calators, reporter molecules, polyamines, polyamides, poly-ethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmaco-dynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/09196, filed Oct. 23, 1992 the entire disclosure of which is incorporated herein by reference. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEES Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Oligonucleotides of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) which is incorporated herein by reference in its entirety.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the instant application, and each of which is herein incorporated by reference.

It is not necessary for all positions in a given compound to be uniformly modified, and in fact more than one of the aforementioned modifications may be incorporated in a single compound or even at a single nucleoside within an oligonucleotide. For example a compound with a modified internucleotide or internucleoside linkage may additionally have modifications of the sugar and/or base. As a further example, a compound with a PNA backbone may have heterocycle modification(s) at one or more positions. The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, increased stability and/or increased binding affinity for the target nucleic acid. An additional region of the oligonucleotide may serve as a substrate for enzymes capable of cleaving RNA:DNA or RNA:RNA hybrids. By way of example, RNase H is a class of cellular endonucleases which cleave the RNA strand of an RNA:DNA duplex. Activation of RNase H, therefore, results in cleavage of the RNA, target, thereby greatly enhancing the efficiency of oligonucleotide inhibition of gene expression. The cleavage of RNA:RNA hybrids can, in like fashion, be accomplished through the actions of endoribonucleases, such as interferon-induced RNAseL which cleaves both cellular and viral RNA. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids, gapped oligonucleotides or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety. Gapped oligonucleotides in which a region of 2′-deoxynucleotides, usually 5 contiguous nucleotides or more, often 10 contiguous deoxynucleotides, is present along with one or two regions of 2′-modified oligonucleotides are often used in antisense technology because uniformly 2′-modified oligonucleotides do not support RNAse H cleavage of the target RNA molecule. Enhanced binding affinity is provided by the 2′ modifications and the deoxy gap region allows RNAse H cleavage of the target. However, in some situations such as modulation of RNA processing as described in the present invention, RNAse H cleavage of the target RNA is not necessary and may be undesired. Consequently, uniformly modified oligonucleotides, i.e., oligonucleotides modified identically at each nucleotide or nucleoside position, are preferred embodiments. Whether or not a given antisense compound is a substrate for RNAse H can be routinely determined using RNAse H assays known in the art. Wu et al., J. Biol. Chem., 1999, 274, 28270-28278; Lima et al., Biochemistry, 1997, 36, 390-398.

A particularly preferred embodiment is an oligonucleotide which is uniformly modified at the 2′ position of the nucleotide sugar, for example with a 2′ MOE, 2′ DMAOE, 2′ guanidinium (U.S. patent application Ser. No. 09/349,040), 2′-O-guanidinium ethyl, 2′ carbamate (U.S. Pat. No. 6,111,085), 2′-dimethylaminoethoxyethoxy (2′ DMAEOE) (U.S. Pat. No. 6,043,352), 2′ aminooxy (U.S. Pat. No. 6,127,533) or 2′ acetamido, particularly N-methyl acetamido (U.S. Pat. No. 6,147,200), modification at each position, or a combination of these. All of these patents are incorporated herein by reference in their entireties.

Other preferred modifications are backbone modifications, including MMI, 3′-methylene phosphonates, morpholino and PNA modifications, which may be uniform or may be alternated with other linkages, particularly phosphodiester or phosphorothioate linkages, as long as RNAse H cleavage is not supported.

In some embodiments, the antisense compound may comprise one or more cationic tails, preferably positively-charged amino acids such as lysine or arginine, conjugated thereto. In a preferred embodiment, the antisense compound comprises one or more peptide nucleic acid linkages with one or more lysine or arginine residues conjugated to the C-terminal end of the molecule. In a preferred embodiment, from 1 to 4 lysine and/or arginine residues are conjugated to each PNA linkage.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The compounds of the invention may be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl)phosphate] derivatives according to the methods disclosed in WO 93/24510 to Gosselin et al., published Dec. 9, 1993 or in WO 94/26764 to Imbach et al.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci., 1977, 66, 1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfoic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The antisense compounds of the present invention can be utilized for diagnostics, therapeutics, prophylaxis and as research reagents and kits. For therapeutics, an animal, preferably a human, suspected of having a disease or disorder which can be treated by modulating the behavior of a cell can be treated by administering antisense compounds in accordance with this invention. The compounds of the invention can be utilized in pharmaceutical compositions by adding an effective amount of an antisense compound to a suitable pharmaceutically acceptable diluent or carrier. Use of the antisense compounds and methods of the invention may also be useful prophylactically, e.g., to prevent or delay infection, inflammation or tumor formation, for example.

The antisense compounds of the invention are useful for research and diagnostics, because these compounds hybridize to nucleic acids encoding a selected mRNA target, enabling sandwich and other assays to easily be constructed to exploit this fact. Hybridization of the antisense oligonucleotides of the invention with a nucleic acid encoding the selected mRNA target can be detected by means known in the art. Such means may include conjugation of an enzyme to the oligonucleotide, radiolabelling of the oligonucleotide or any other suitable detection means. Kits using such detection means for detecting the level of target in a sample may also be prepared.

The present invention also includes pharmaceutical compositions and formulations which include the antisense compounds of the invention. The pharmaceutical compositions of, the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification, including chimeric molecules or molecules which may have a 2′-O-methoxyethyl modification of every nucleotide sugar, are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

Emulsions

The compositions of the present invention may be prepared and formulated as emulsions. Emulsions are typically heterogenous systems of one liquid dispersed in another in the form of droplets usually exceeding 0.1 μm in diameter. (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising of two immiscible liquid phases intimately mixed and dispersed with each other. In general, emulsions may be either water-in-oil (w/o) or of the oil-in-water (o/w) variety. When an aqueous phase is finely divided into and dispersed as minute droplets into a bulk oily phase the resulting composition is called a water-in-oil (w/o) emulsion. Alternatively, when an oily phase is finely divided into and dispersed as minute droplets into a bulk aqueous phase the resulting composition is called an oil-in-water (o/w) emulsion. Emulsions may contain additional components in addition to the dispersed phases and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199.

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of reasons of ease of formulation, efficacy from an absorption and bioavailability standpoint. (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment of the present invention, the compositions of oligonucleotides and nucleic acids are formulated as microemulsions. A microemulsion may be defined as a system of water, oil and amphiphile which is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C₈-C₁₂) mono, di, and tri-glycerides, polyoxyethylated glyceryl fatty acid esters, fatty alcohols, polyglycolized glycerides, saturated polyglycolized C₈-C₁₀ glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile drugs, peptides or oligonucleotides. Microemulsions have also been effective in the transdermal delivery of active components in both cosmetic and pharmaceutical applications. It is expected that the microemulsion compositions and formulations of the present invention will facilitate the increased systemic absorption of oligonucleotides and nucleic acids from the gastrointestinal tract, as well as improve the local cellular uptake of oligonucleotides and nucleic acids within the gastrointestinal tract, vagina, buccal cavity and other areas of administration.

Microemulsions of the present invention may also contain additional components and additives such as sorbitan monostearate (Grill 3), Labrasol, and penetration enhancers to improve the properties of the formulation and to enhance the absorption of the oligonucleotides and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

Liposomes

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

In order to cross intact mammalian skin, lipid vesicles must pass through a series of fine pores, each with a diameter less than 50 nm, under the influence of a suitable transdermal gradient. Therefore, it is desirable to use a liposome which is highly deformable and able to pass through such fine pores.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes. As the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis.

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g. as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al. S.T.P. Pharma. Sci., 1994, 4, 6, 466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765). Various liposomes comprising one or more glycolipids are known in the art. Papahadjopoulos et al. (Ann. N.Y. Acad. Sci., 1987, 507, 64) reported the ability of monosialoganglioside G_{M1}, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes comprising (1) sphingomyelin and (2) the ganglioside G_{M1} or a galactocerebroside sulfate ester. U.S. Pat. No. 5,543,152 discloses liposomes comprising sphingomyelin. Liposomes comprising 1,2-sn-dimyristoylphosphatidylcholine are disclosed in WO 97/13499.

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C₁₂15G, that contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described in U.S. Pat. Nos. 4,426,330 and 4,534,899. Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidyl-ethanolamine (DSPE) and PEG. Liposomes having covalently bound PEG moieties on their external surface are described in European Patent EP 0 445 131 B1 and PCT WO90/04384.

Liposome compositions containing 1-20 mole percent of PE derivatized with PEG, and methods of use thereof, are described in U.S. Pat. Nos. 5,013,556, 5,356,633, 5,213,804 and European Patent 0 496 813 B1. Liposomes comprising a number of other lipid-polymer conjugates are disclosed in WO 91/05545 and U.S. Pat. No. 5,225,212 and in WO 94/20073 Liposomes comprising PEG-modified ceramide lipids are described in WO 96/10391. U.S. Pat. Nos. 5,540,935 and 5,556,948 describe PEG-containing liposomes that can be further derivatized with functional moieties on their surfaces.

A limited number of liposomes comprising nucleic acids are known in the art. WO 96/40062 discloses methods for encapsulating high molecular weight nucleic acids in liposomes. U.S. Pat. No. 5,264,221 discloses protein-bonded liposomes and asserts that the contents of such liposomes may include an antisense RNA. U.S. Pat. No. 5,665,710 describes certain methods of encapsulating oligodeoxynucleotides in liposomes. PCT WO97/04787 discloses liposomes comprising antisense oligonucleotides targeted to the raf gene.

Transfersomes are yet another type of liposomes, and are highly deformable lipid aggregates which are attractive candidates for drug delivery vehicles. Transfersomes may be described as lipid droplets which are so highly deformable that they are easily able to penetrate through pores which are smaller than the droplet. Transfersomes are adaptable to the environment in which they are used, e.g. they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, N.Y., 1988, p. 285).

Penetration Enhancers

In one embodiment, the present invention employs various penetration enhancers to effect the efficient delivery of nucleic acids, particularly oligonucleotides, to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers may be classified as belonging to one of five broad categories, i.e., surfactants, fatty acids, bile salts, chelating agents, and non-chelating non-surfactants (Lee et al., Critical Reviews in Therapeutic Drug Carrier Systems, 1991, p. 92). Each of the above mentioned classes of penetration enhancers are described below in greater detail.

Surfactants: In connection with the present invention, surfactants (or “surface-active agents”) are chemical entities which, when dissolved in an aqueous solution, reduce the surface tension of the solution or the interfacial tension between the aqueous solution and another liquid, with the result that absorption of oligonucleotides through the mucosa is enhanced. In addition to bile salts and fatty acids, these penetration enhancers include, for example, sodium lauryl sulfate, polyoxyethylene-9-lauryl ether and polyoxyethylene-20-cetyl ether) (Lee et al., Critical Reviews in Therapeutic Drug Carrier Systems, 1991, p. 92); and perfluorochemical emulsions, such as FC-43. Takahashi et al., J. Pharm. Pharmacol., 1988, 40, 252).

Fatty acids: Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic, acid, glycerol 1-monocaprate, 1-dodecylazacycloheptan-2-one, acylcarnitines, acylcholines, C₁₋₁₀ alkyl esters thereof (e.g., methyl, isopropyl and t-butyl), and mono- and di-glycerides thereof (i.e., oleate, laurate, caprate, myristate, palmitate, stearate, linoleate, etc.) (Lee et al., Critical Reviews in Therapeutic Drug Carrier Systems, 1991, p. 92; Muranishi, Critical Reviews in Therapeutic Drug Carrier Systems, 1990, 7, 1-33; El Hariri et al., J. Pharm. Pharmacol., 1992, 44, 651-654

Bile salts: The physiological role of bile includes the facilitation of dispersion and absorption of lipids and fat-soluble vitamins (Brunton, Chapter 38 in: Goodman & Gilman's The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating Agents Chelating agents, as used in connection with the present invention, can be defined as compounds that remove metallic ions from solution by forming complexes therewith, with the result that absorption of oligonucleotides through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Chelating agents of the invention include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines) (Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14, 43-51).

Non-chelating non-surfactants: As used herein, non-chelating non-surfactant penetration enhancing compounds can be defined as compounds that demonstrate insignificant activity as chelating agents or as surfactants but that nonetheless enhance absorption of oligonucleotides through the alimentary mucosa (Muranishi, Critical Reviews in Therapeutic Drug Carrier Systems, 1990, 7, 1-33). This class of penetration enhancers include, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621-626).

Agents that enhance uptake of oligonucleotides at the cellular level may also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of oligonucleotides.

Other agents may be utilized to enhance the penetration of the administered nucleic acids, including glycols such as ethylene glycol and propylene glycol, pyrrols such as 2-pyrrol, azones, and terpenes such as limonene and menthone.

Carriers

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate oligonucleotide in hepatic tissue can be reduced when it is coadministered with polyinosinic acid, dextran sulfate, polycytidic acid or 4-acetamido-4′ isothiocyano-stilbene-2,2′-disulfonic acid (Miyao et al., Antisense Res. Dev., 1995, 5, 115-121; Takakura et al., Antisense & Nucl. Acid Drug Dev., 1996, 6, 177-183).

Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipient suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

Other Components

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances which increase the viscosity of the suspension including, for example, sodium carboxymethylcellulose, sorbitol and/or dextran. The suspension may also contain stabilizers.

Certain embodiments of the invention provide pharmaceutical compositions containing (a) one or more antisense compounds and (b) one or more other chemotherapeutic agents which function by a non-antisense mechanism. Examples of such chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES). See, generally, The Merck Manual of Diagnosis and Therapy, 15th Ed., Berkow et al., eds., 1987, Rahway, N.J., pages 1206-1228). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. See, generally, The Merck Manual of Diagnosis and Therapy, 15th Ed., Berkow et al., eds., 1987, Rahway, N.J., pages 2499-2506 and 46-49, respectively). Other non-antisense chemotherapeutic agents are also within the scope of this invention. Two or more combined compounds may be used together or sequentially.

In another related embodiment, compositions of the invention may contain one or more antisense compounds, particularly oligonucleotides, targeted to a first nucleic acid and one or more additional antisense compounds targeted to a second nucleic acid target. Numerous examples of antisense compounds are known in the art. Two or more combined compounds may be used together or sequentially.

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models. In general, dosage is from 0.0001 μg to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.0001 μg to 100 g per kg of body weight, once or more daily, to once every 20 years.

While the present invention has been described with specificity in accordance with certain of its preferred embodiments, the following examples serve only to illustrate the invention and are not intended to limit the same.

### EXAMPLES

### Example 1

Nucleoside Phosphoramidites for Oligonucleotide Synthesis Deoxy and 2′-alkoxy amidites

2′-Deoxy and 2′-methoxy beta-cyanoethyldiisopropyl phosphoramidites were purchased from commercial sources (e.g. Chemgenes, Needham Mass. or Glen Research, Inc. Sterling Va.). Other 2′-O-alkoxy substituted nucleoside amidites are prepared as described in U.S. Pat. No. 5,506,351, herein incorporated by reference. For oligonucleotides synthesized using 2′-alkoxy amidites, optimized synthesis cycles were developed that incorporate multiple steps coupling longer wait times relative to standard synthesis cycles.

The following abbreviations are used in the text: thin layer chromatography (TLC), melting point (MP), high pressure liquid chromatography (HPLC), Nuclear Magnetic Resonance (NMR), argon (Ar), methanol (MeOH), dichloromethane (CH₂Cl₂), triethylamine (TEA), dimethyl formamide (DMF), ethyl acetate (EtOAc), dimethyl sulfoxide (DMSO), tetrahydrofuran (THF).

Oligonucleotides containing 5-methyl-2′-deoxycytidine (5-Me-dC) nucleotides were synthesized according to published methods (Sanghvi, et. al., Nucleic Acids Research, 1993, 21, 3197-3203) using commercially available phosphoramidites (Glen Research, Sterling Va. or ChemGenes, Needham Mass.) or prepared as follows:

### Preparation of 5′-O-Dimethoxytrityl-thymidine intermediate for 5-methyl dC amidite

To a 50 L glass reactor equipped with air stirrer and Ar gas line was added thymidine (1.00 kg, 4.13 mol) in anhydrous pyridine (6 L) at ambient temperature. Dimethoxytrityl (DMT) chloride (1.47 kg, 4.34 mol, 1.05 eq) was added as a solid in four portions over 1 h. After 30 min, TLC indicated approx. 95% product, 2% thymidine, 5% DMT reagent and by-products and 2% 3′,5′-bis DMT product (R_{f} in EtOAc 0.45, 0.05, 0.98, 0.95 respectively). Saturated sodium bicarbonate (4 L) and CH₂Cl₂ were added with stirring (pH of the aqueous layer 7.5). An additional 18 L of water was added, the mixture was stirred, the phases were separated, and the organic layer was transferred to a second 50 L vessel. The aqueous layer was extracted with additional CH₂Cl₂ (2×2 L). The combined organic layer was washed with water (10 L) and then concentrated in a rotary evaporator to approx. 3.6 kg total weight. This was redissolved in CH₂Cl₂ (3.5 L), added to the reactor followed by water (6 L) and hexanes (13 L). The mixture was vigorously stirred and seeded to give a fine white suspended solid starting at the interface. After stirring for 1 h, the suspension was removed by suction through a ½″ diameter teflon tube into a 20 L suction flask, poured onto a 25 cm Coors Buchner funnel, washed with water (2×3 L) and a mixture of hexanes-CH₂Cl₂ (4:1, 2×3 L) and allowed to air dry overnight in pans (1″ deep). This was further dried in a vacuum oven (75° C., 0.1 mm Hg, 48 h) to a constant weight of 2072 g (93%) of a white solid, (mp 122-124° C.). TLC indicated a trace contamination of the bis DMT product. NMR spectroscopy also indicated that 1-2 mole percent pyridine and about 5 mole percent of hexanes was still present.

### Preparation of 5′-O-Dimethoxytrityl-2′-deoxy-5-methylcytidine intermediate for 5-methyl-dC amidite

To a 50 L Schott glass-lined steel reactor equipped with an electric stirrer, reagent addition pump (connected to an addition funnel), heating/cooling system, internal thermometer and an Ar gas line was added 5′-O-dimethoxytrityl-thymidine (3.00 kg, 5.51 mol), anhydrous acetonitrile (25 L) and TEA (12.3 L, 88.4 mol, 16 eq). The mixture was chilled with stirring to −10° C. internal temperature (external −20° C.). Trimethylsilylchloride (2.1 L, 16.5 mol, 3.0 eq) was added over 30 minutes while maintaining the internal temperature below −5° C., followed by a wash of anhydrous acetonitrile (1 L). Note: the reaction is mildly exothermic and copious hydrochloric acid fumes form over the course of the addition. The reaction was allowed to warm to 0° C. and the reaction progress was confirmed by TLC (EtOAc-hexanes 4:1; R_{f} 0.43 to 0.84 of starting material and silyl product, respectively). Upon completion, triazole (3.05 kg, 44 mol, 8.0 eq) was added the reaction was cooled to −20° C. internal temperature (external −30° C.). Phosphorous oxychloride (1035 mL, 11.1 mol, 2.01 eq) was added over 60 min so as to maintain the temperature between −20° C. and −10° C. during the strongly exothermic process, followed by a wash of anhydrous acetonitrile (1 L). The reaction was warmed to 0° C. and stirred for 1 h. TLC indicated a complete conversion to the triazole product (R_{f} 0.83 to 0.34 with the product spot glowing in long wavelength UV light). The reaction mixture was a peach-colored thick suspension, which turned darker red upon warming without apparent decomposition. The reaction was cooled to −15° C. internal temperature and water (5 L) was slowly added at a rate to maintain the temperature below +10° C. in order to quench the reaction and to form a homogenous solution. (Caution: this reaction is initially very strongly exothermic). Approximately one-half of the reaction volume (22 L) was transferred by air pump to another vessel, diluted with EtOAc (12 L) and extracted with water (2×8 L). The combined water layers were back-extracted with EtOAc (6 L). The water layer was discarded and the organic layers were concentrated in a 20 L rotary evaporator to an oily foam. The foam was coevaporated with anhydrous acetonitrile (4 L) to remove EtOAc. (note: dioxane may be used instead of anhydrous acetonitrile if dried to a hard foam). The second half of the reaction was treated in the same way. Each residue was dissolved in dioxane (3 L) and concentrated ammonium hydroxide (750 mL) was added. A homogenous solution formed in a few minutes and the reaction was allowed to stand overnight (although the reaction is complete within 1 h).

TLC indicated a complete reaction (product R_{f} 0.35 in EtOAc-MeOH 4:1). The reaction solution was concentrated on a rotary evaporator to a dense foam. Each foam was slowly redissolved in warm EtOAc (4 L; 50° C.), combined in a 50 L glass reactor vessel, and extracted with water (2×4 L) to remove the triazole by-product. The water was back-extracted with EtOAc (2 L). The organic layers were combined and concentrated to about 8 kg total weight, cooled to 0° C. and seeded with crystalline product. After 24 hours, the first crop was collected on a 25 cm Coors Buchner funnel and washed repeatedly with EtOAc (3×3 L) until a white powder was left and then washed with ethyl ether (2×3 L). The solid was put in pans (1″ deep) and allowed to air dry overnight. The filtrate was concentrated to an oil, then redissolved in EtOAc (2 L), cooled and seeded as before. The second crop was collected and washed as before (with proportional solvents) and the filtrate was first extracted with water (2×1 L) and then concentrated to an oil. The residue was dissolved in EtOAc (1 L) and yielded a third crop which was treated as above except that more washing was required to remove a yellow oily layer.

After air-drying, the three crops were dried in a vacuum oven (50° C., 0.1 mm Hg, 24 h) to a constant weight (1750, 600 and 200 g, respectively) and combined to afford 2550 g (85%) of a white crystalline product (MP 215-217° C.) when TLC and NMR spectroscopy indicated purity. The mother liquor still contained mostly product (as determined by TLC) and a small amount of triazole (as determined by NMR spectroscopy), bis DMT product and unidentified minor impurities. If desired, the mother liquor can be purified by silica gel chromatography using a gradient of MeOH (0-25%) in EtOAc to further increase the yield.

### Preparation of 5′-O-Dimethoxytrityl-2′-deoxy-N4-benzoyl-5-methylcytidine penultimate intermediate for 5-methyl dC amidite

Crystalline 5′-O-dimethoxytrityl-5-methyl-2′-deoxycytidine (2000 g, 3.68 mol) was dissolved in anhydrous DMF (6.0 kg) at ambient temperature in a 50 L glass reactor vessel equipped with an air stirrer and argon line. Benzoic anhydride (Chem Impex not Aldrich, 874 g, 3.86 mol, 1.05 eq) was added and the reaction was stirred at ambient temperature for 8 h. TLC (CH₂Cl₂-EtOAc; CH₂Cl₂-EtOAc 4:1; R_{f} 0.25) indicated approx. 92% complete reaction. An additional amount of benzoic anhydride (44 g, 0.19 mol) was added. After a total of 18 h, TLC indicated approx. 96% reaction completion. The solution was diluted with EtOAc (20 L), TEA (1020 mL, 7.36 mol, ca 2.0 eq) was added with stirring, and the mixture was extracted with water (15 L, then 2×10 L). The aqueous layer was removed (no back-extraction was needed) and the organic layer was concentrated in 2×20 L rotary evaporator flasks until a foam began to form. The residues were coevaporated with acetonitrile (1.5 L each) and dried (0.1 mm Hg, 25° C., 24 h) to 2520 g of a dense foam. High pressure liquid chromatography (HPLC) revealed a contamination of 6.3% of N4,3′-O-dibenzoyl product, but very little other impurities.

The product was purified by Biotage column chromatography (5 kg Biotage) prepared with 65:35:1 hexanes-EtOAc-TEA (4 L). The crude product (800 g), dissolved in CH₂Cl₂ (2 L), was applied to the column. The column was washed with the 65:35:1 solvent mixture (20 kg), then 20:80:1 solvent mixture (10 kg), then 99:1 EtOAc:TEA (17 kg). The fractions containing the product were collected, and any fractions containing the product and impurities were retained to be resubjected to column chromatography. The column was re-equilibrated with the original 65:35:1 solvent mixture (17 kg). A second batch of crude product (840 g) was applied to the column as before. The column was washed with the following solvent gradients: 65:35:1 (9 kg), 55:45:1 (20 kg), 20:80:1 (10 kg), and 99:1 EtOAc:TEA (15 kg). The column was reequilibrated as above, and a third batch of the crude product (850 g) plus impure fractions recycled from the two previous columns (28 g) was purified following the procedure for the second batch. The fractions containing pure product combined and concentrated on a 20 L rotary evaporator, co-evaporated with acetontirile (3 L) and dried (0.1 mm Hg, 48 h, 25° C.) to a constant weight of 2023 g (85%) of white foam and 20 g of slightly contaminated product from the third run. HPLC indicated a purity of 99.8% with the balance as the diBenzoyl product.

### [5′-O-(4,4′-Dimethoxytriphenylmethyl)-2′-deoxy-N⁴-benzoyl-5-methylcytidin-3′-O-yl]-2-cyanoethyl-N,N-diisopropylphosphoramidite (5-methyl dC amidite)

5′-O-(4,4′-Dimethoxytriphenylmethyl)-2′-deoxy-N⁴-benzoyl-5-methylcytidine (998 g, 1.5 mol) was dissolved in anhydrous DMF (2 L). The solution was co-evaporated with toluene (300 ml) at 50° C. under reduced pressure, then cooled to room temperature and 2-cyanoethyl tetraisopropylphosphorodiamidite (680 g, 2.26 mol) and tetrazole (52.5 g, 0.75 mol) were added. The mixture was shaken until all tetrazole was dissolved, N-methylimidazole (15 ml) was added and the mixture was left at room temperature for 5 hours. TEA (300 ml) was added, the mixture was diluted with DMF (2.5 L) and water (600 ml), and extracted with hexane (3×3 L). The mixture was diluted with water (1.2 L) and extracted with a mixture of toluene (7.5 L) and hexane (6 L). The two layers were separated, the upper layer was washed with DMF-water (7:3 v/v, 3×2 L) and water (3×2 L), and the phases were separated. The organic layer was dried (Na₂SO₄), filtered and rotary evaporated. The residue was co-evaporated with acetonitrile (2×2 L) under reduced pressure and dried to a constant weight (25° C., 0.1 mm Hg, 40 h) to afford 1250 g an off-white foam solid (96%).

### 2′-Fluoro amidites

### 2′-Fluorodeoxyadenosine amidites

2′-fluoro oligonucleotides were synthesized as described previously [Kawasaki, et. al., J. Med. Chem., 1993, 36, 831-841] and U.S. Pat. No. 5,670,633, herein incorporated by reference. The preparation of 2′-fluoropyrimidines containing a 5-methyl substitution are described in U.S. Pat. No. 5,861,493. Briefly, the protected nucleoside N6-benzoyl-2′-deoxy-2′-fluoroadenosine was synthesized utilizing commercially available 9-beta-D-arabinofuranosyladenine as starting material and whereby the 2′-alpha-fluoro atom is introduced by a S_{N}2-displacement of a 2′-beta-triflate group. Thus N6-benzoyl-9-beta-D-arabinofuranosyladenine was selectively protected in moderate yield as the 3′,5′-ditetrahydropyranyl (THP) intermediate. Deprotection of the THP and N6-benzoyl groups was accomplished using standard methodologies to obtain the 5′-dimethoxytrityl-(DMT) and 5′-DMT-3′-phosphoramidite intermediates.

### 2′-Fluorodeoxyguanosine

The synthesis of 2′-deoxy-2′-fluoroguanosine was accomplished using tetraisopropyldisiloxanyl (TPDS) protected 9-beta-D-arabinofuranosylguanine as starting material, and conversion to the intermediate isobutyryl-arabinofuranosylguanosine. Alternatively, isobutyryl-arabinofuranosylguanosine was prepared as described by Ross et al., (Nucleosides & Nucleosides, 16, 1645, 1997). Deprotection of the TPDS group was followed by protection of the hydroxyl group with THP to give isobutyryl di-THP protected arabinofuranosylguanine. Selective O-deacylation and triflation was followed by treatment of the crude product with fluoride, then deprotection of the THP groups. Standard methodologies were used to obtain the 5′-DMT- and 5′-DMT-3′-phosphoramidites.

### 2′-Fluorouridine

Synthesis of 2′-deoxy-2′-fluorouridine was accomplished by the modification of a literature procedure in which 2,2′-anhydro-1-beta-D-arabinofuranosyluracil was treated with 70% hydrogen fluoride-pyridine. Standard procedures were used to obtain the 5′-DMT and 5′-DMT-3′ phosphoramidites.

### 2′-Fluorodeoxycytidine

2′-deoxy-2′-fluorocytidine was synthesized via amination of 2′-deoxy-2′-fluorouridine, followed by selective protection to give N4-benzoyl-2′-deoxy-2′-fluorocytidine. Standard procedures were used to obtain the 5′-DMT and 5′-DMT-3′ phosphoramidites.

### 2′-O-(2-Methoxyethyl) modified amidites

2′-O-Methoxyethyl-substituted nucleoside amidites (otherwise known as MOE amidites) are prepared as follows, or alternatively, as per the methods of Martin, P., (Helvetica Chimica Acta, 1995, 78, 486-504).

### Preparation of 2′-O-(2-methoxyethyl)-5-methyluridine intermediate

2,2′-Anhydro-5-methyl-uridine (2000 g, 8.32 mol), tris(2-methoxyethyl)borate (2504 g, 10.60 mol), sodium bicarbonate (60 g, 0.70 mol) and anhydrous 2-methoxyethanol (5 L) were combined in a 12 L three necked flask and heated to 130° C. (internal temp) at atmospheric pressure, under an argon atmosphere with stirring for 21 h. TLC indicated a complete reaction. The solvent was removed under reduced pressure until a sticky gum formed (50-85° C. bath temp and 100-11 mm Hg) and the residue was redissolved in water (3 L) and heated to boiling for 30 min in order the hydrolyze the borate esters. The water was removed under reduced pressure until a foam began to form and then the process was repeated. HPLC indicated about 77% product, 15% dimer (5′ of product attached to 2′ of starting material) and unknown derivatives, and the balance was a single unresolved early eluting peak.

The gum was redissolved in brine (3 L), and the flask was rinsed with additional brine (3 L). The combined aqueous solutions were extracted with chloroform (20 L) in a heavier-than continuous extractor for 70 h. The chloroform layer was concentrated by rotary evaporation in a 20 L flask to a sticky foam (2400 g). This was coevaporated with MeOH (400 mL) and EtOAc (8 L) at 75° C. and 0.65 atm until the foam dissolved at which point the vacuum was lowered to about 0.5 atm. After 2.5 L of distillate was collected a precipitate began to form and the flask was removed from the rotary evaporator and stirred until the suspension reached ambient temperature. EtOAc (2 L) was added and the slurry was filtered on a 25 cm table top Buchner funnel and the product was washed with EtOAc (3×2 L). The bright white solid was air dried in pans for 24 h then further dried in a vacuum oven (50° C., 0.1 mm Hg, 24 h) to afford 1649 g of a white crystalline solid (mp 115.5-116.5° C.).

The brine layer in the 20 L continuous extractor was further extracted for 72 h with recycled chloroform. The chloroform was concentrated to 120 g of oil and this was combined with the mother liquor from the above filtration (225 g), dissolved in brine (250 mL) and extracted once with chloroform (250 mL). The brine solution was continuously extracted and the product was crystallized as described above to afford an additional 178 g of crystalline product containing about 2% of thymine. The combined yield was 1827 g (69.4%). HPLC indicated about 99.5% purity with the balance being the dimer.

### Preparation of 5′-O-DMT-2′-O-(2-methoxyethyl)-5-methyluridine penultimate intermediate

In a 50 L glass-lined steel reactor, 2′-O-(2-methoxyethyl)-5-methyl-uridine (MOE-T, 1500 g, 4.738 mol), lutidine (1015 g, 9.476 mol) were dissolved in anhydrous acetonitrile (15 L). The solution was stirred rapidly and chilled to −10° C. (internal temperature). Dimethoxytriphenylmethyl chloride (1765.7 g, 5.21 mol) was added as a solid in one portion. The reaction was allowed to warm to −2° C. over 1 h. (Note: The reaction was monitored closely by TLC (EtOAc) to determine when to stop the reaction so as to not generate the undesired bis-DMT substituted side product). The reaction was allowed to warm from −2 to 3° C. over 25 min. then quenched by adding MeOH (300 mL) followed after 10 min by toluene (16 L) and water (16 L). The solution was transferred to a clear 50 L vessel with a bottom outlet, vigorously stirred for 1 minute, and the layers separated. The aqueous layer was removed and the organic layer was washed successively with 10% aqueous citric acid (8 L) and water (12 L). The product was then extracted into the aqueous phase by washing the toluene solution with aqueous sodium hydroxide (0.5N, 16 L and 8 L). The combined aqueous layer was overlayed with toluene (12 L) and solid citric acid (8 moles, 1270 g) was added with vigorous stirring to lower the pH of the aqueous layer to 5.5 and extract the product into the toluene. The organic layer was washed with water (10 L) and TLC of the organic layer indicated a trace of DMT-O-Me, bis DMT and dimer DMT.

The toluene solution was applied to a silica gel column (6 L sintered glass funnel containing approx. 2 kg of silica gel slurried with toluene (2 L) and TEA (25 mL)) and the fractions were eluted with toluene (12 L) and EtOAc (3×4 L) using vacuum applied to a filter flask placed below the column. The first EtOAc fraction containing both the desired product and impurities were resubjected to column chromatography as above. The clean fractions were combined, rotary evaporated to a foam, coevaporated with acetonitrile (6 L) and dried in a vacuum oven (0.1 mm Hg, 40 h, 40° C.) to afford 2850 g of a white crisp foam. NMR spectroscopy indicated a 0.25 mole % remainder of acetonitrile (calculates to be approx. 47 g) to give a true dry weight of 2803 g (96%). HPLC indicated that the product was 99.41% pure, with the remainder being 0.06 DMT-O-Me, 0.10 unknown, 0.44 bis DMT, and no detectable dimer DMT or 3′-O-DMT.

### Preparation of [5′-O-(4,4′-Dimethoxytriphenylmethyl)-2′-O-(2-methoxyethyl)-5-methyluridin-3′-O-yl]-2-cyanoethyl-N,N-diisopropylphosphoramidite T amidite)

5′-O-(4,4′-Dimethoxytriphenylmethyl)-2′-O-(2-methoxyethyl)-5-methyluridine (1237 g, 2.0 mol) was dissolved in anhydrous DMF (2.5 L). The solution was co-evaporated with toluene (200 ml) at 50° C. under reduced pressure, then cooled to room temperature and 2-cyanoethyl tetraisopropylphosphorodiamidite (900 g, 3.0 mol) and tetrazole (70 g, 1.0 mol) were added. The mixture was shaken until all tetrazole was dissolved, N-methylimidazole (20 ml) was added and the solution was left at room temperature for 5 hours. TEA (300 ml) was added, the mixture was diluted with DMF (3.5 L) and water (600 ml) and extracted with hexane (3×3 L). The mixture was diluted with water (1.6 L) and extracted with the mixture of toluene (12 L) and hexanes (9 L). The upper layer was washed with DMF-water (7:3 v/v, 3×3 L) and water (3×3 L). The organic layer was dried (Na₂SO₄), filtered and evaporated. The residue was co-evaporated with acetonitrile (2×2 L) under reduced pressure and dried in a vacuum oven (25° C., 0.1 mm Hg, 40 h) to afford 1526 g of an off-white foamy solid (95%).

### Preparation of 5′-O-Dimethoxytrityl-2′-O-(2-methoxyethyl)-5-methylcytidine intermediate

To a 50 L Schott glass-lined steel reactor equipped with an electric stirrer, reagent addition pump (connected to an addition funnel), heating/cooling system, internal thermometer and argon gas line was added 5′-O-dimethoxytrityl-2′-O-(2-methoxyethyl)-5-methyl-uridine (2.616 kg, 4.23 mol, purified by base extraction only and no scrub column), anhydrous acetonitrile (20 L), and TEA (9.5 L, 67.7 mol, 16 eq). The mixture was chilled with stirring to −10° C. internal temperature (external −20° C.) Trimethylsilylchloride (1.60 L, 12.7 mol, 3.0 eq) was added over 30 min. while maintaining the internal temperature below −5° C., followed by a wash of anhydrous acetonitrile (1 L). (Note: the reaction is mildly exothermic and copious hydrochloric acid fumes form over the course of the addition). The reaction was allowed to warm to 0° C. and the reaction progress was confirmed by TLC (EtOAc, R_{f} 0.68 and 0.87 for starting material and silyl product, respectively). Upon completion, triazole (2.34 kg, 33.8 mol, 8.0 eq) was added the reaction was cooled to −20° C. internal temperature (external −30° C.). Phosphorous oxychloride (793 mL, 8.51 mol, 2.01 eq) was added slowly over 60 min so as to maintain the temperature between −20° C. and −10° C. (note: strongly exothermic), followed by a wash of anhydrous acetonitrile (1 L). The reaction was warmed to 0° C. and stirred for 1 h, at which point it was an off-white thick suspension. TLC indicated a complete conversion to the triazole product (EtOAc, R_{f} 0.87 to 0.75 with the product spot glowing in long wavelength UV light). The reaction was cooled to −15° C. and water (5 L) was slowly added at a rate to maintain the temperature below +10° C. in order to quench the reaction and to form a homogenous solution. (Caution: this reaction is initially very strongly exothermic). Approximately one-half of the reaction volume (22 L) was transferred by air pump to another vessel, diluted with EtOAc (12 L) and extracted with water (2×8 L). The second half of the reaction was treated in the same way. The combined aqueous layers were back-extracted with EtOAc (8 L) The organic layers were combined and concentrated in a 20 L rotary evaporator to an oily foam. The foam was coevaporated with anhydrous acetonitrile (4 L) to remove EtOAc. (note: dioxane may be used instead of anhydrous acetonitrile if dried to a hard foam). The residue was dissolved in dioxane (2 L) and concentrated ammonium hydroxide (750 mL) was added. A homogenous solution formed in a few minutes and the reaction was allowed to stand overnight

TLC indicated a complete reaction (CH₂Cl₂-acetone-MeOH, 20:5:3, R_{f} 0.51). The reaction solution was concentrated on a rotary evaporator to a dense foam and slowly redissolved in warm CH₂Cl₂ (4 L, 40° C.) and transferred to a 20 L glass extraction vessel equipped with a air-powered stirrer. The organic layer was extracted with water (2×6 L) to remove the triazole by-product. (Note: In the first extraction an emulsion formed which took about 2 h to resolve). The water layer was back-extracted with CH₂Cl₂ (2×2 L), which in turn was washed with water (3 L). The combined organic layer was concentrated in 2×20 L flasks to a gum and then recrystallized from EtOAc seeded with crystalline product. After sitting overnight, the first crop was collected on a 25 cm Coors Buchner funnel and washed repeatedly with EtOAc until a white free-flowing powder was left (about 3×3 L). The filtrate was concentrated to an oil recrystallized from EtOAc, and collected as above. The solid was air-dried in pans for 48 h, then further dried in a vacuum oven (50° C., 0.1 mm Hg, 17 h) to afford 2248 g of a bright white, dense solid (86%). An HPLC analysis indicated both crops to be 99.4% pure and NMR spectroscopy indicated only a faint trace of EtOAc remained.

### Preparation of 5′-O-dimethoxytrityl-2′-O-(2-methoxyethyl)-N4-benzoyl-5-methyl-cytidine penultimate intermediate

Crystalline 5′-O-dimethoxytrityl-2′-O-(2-methoxyethyl)-5-methyl-cytidine (1000 g, 1.62 mol) was suspended in anhydrous DMF (3 kg) at ambient temperature and stirred under an Ar atmosphere. Benzoic anhydride (439.3 g, 1.94 mol) was added in one portion. The solution clarified after 5 hours and was stirred for 16 h. HPLC indicated 0.45% starting material remained (as well as 0.32% N4,3′-O-bis Benzoyl). An additional amount of benzoic anhydride (6.0 g, 0.0265 mol) was added and after 17 h, HPLC indicated no starting material was present. TEA (450 mL, 3.24 mol) and toluene (6 L) were added with stirring for 1 minute. The solution was washed with water (4×4 L), and brine (2×4 L). The organic layer was partially evaporated on a 20 L rotary evaporator to remove 4 L of toluene and traces of water. HPLC indicated that the bis benzoyl side product was present as a 6% impurity. The residue was diluted with toluene (7 L) and anhydrous DMSO (200 mL, 2.82 mol) and sodium hydride (60% in oil, 70 g, 1.75 mol) was added in one portion with stirring at ambient temperature over 1 h. The reaction was quenched by slowly adding then washing with aqueous citric acid (10%, 100 mL over 10 min, then 2×4 L), followed by aqueous sodium bicarbonate (2%, 2 L), water (2×4 L) and brine (4 L). The organic layer was concentrated on a 20 L rotary evaporator to about 2 L total volume. The residue was purified by silica gel column chromatography (6 L Buchner funnel containing 1.5 kg of silica gel wetted with a solution of EtOAc-hexanes-TEA (70:29:1)). The product was eluted with the same solvent (30 L) followed by straight EtOAc (6 L). The fractions containing the product were combined, concentrated on a rotary evaporator to a foam and then dried in a vacuum oven (50° C., 0.2 mm Hg, 8 h) to afford 1155 g of a crisp, white foam (98%). HPLC indicated a purity of >99.7%.

### Preparation of [5′-O-(4,4′-Dimethoxytriphenylmethyl)-2′-O-(2-methoxyethyl)-N⁴-benzoyl-5-methylcytidin-3′-O-yl]-2-cyanoethyl-N,N-diisopropylphosphoramidite (MOE 5-Me-C amidite)

5′-O-(4,4′-Dimethoxytriphenylmethyl)-2′-O-(2-methoxyethyl)-N⁴-benzoyl-5-methylcytidine (1082 g, 1.5 mol) was dissolved in anhydrous DMF (2 L) and co-evaporated with toluene (300 ml) at 50° C. under reduced pressure. The mixture was cooled to room temperature and 2-cyanoethyl tetraisopropylphosphorodiamidite (680 g, 2.26 mol) and tetrazole (52.5 g, 0.75 mol) were added. The mixture was shaken until all tetrazole was dissolved, N-methylimidazole (30 ml) was added, and the mixture was left at room temperature for 5 hours. TEA (300 ml) was added, the mixture was diluted with DMF (1 L) and water (400 ml) and extracted with hexane (3×3 L). The mixture was diluted with water (1.2 L) and extracted with a mixture of toluene (9 L) and hexanes (6 L). The two layers were separated and the upper layer was washed with DMF-water (60:40 v/v, 3×3 L) and water (3×2 L). The organic layer was dried (Na₂SO₄), filtered and evaporated. The residue was co-evaporated with acetonitrile (2×2 L) under reduced pressure and dried in a vacuum oven (25° C., 0.1 mm Hg, 40 h) to afford 1336 g of an off-white foam (97%).

### Preparation of [5′-O-(4,4′-Dimethoxytriphenylmethyl)-2′-O-(2-methoxyethyl)-N⁶-benzoyladenosin-3′-O-yl]-2-cyanoethyl-N,N-diisopropylphosphoramidite (MOE A amidite)

5′-O-(4,4′-Dimethoxytriphenylmethyl)-2′-O-(2-methoxyethyl)-N⁶-benzoyladenosine (purchased from Reliable Biopharmaceutical, St. Lois, Mo.), 1098 g, 1.5 mol) was dissolved in anhydrous DMF (3 L) and co-evaporated with toluene (300 ml) at 50° C. The mixture was cooled to room temperature and 2-cyanoethyl tetraisopropylphosphorodiamidite (680 g, 2.26 mol) and tetrazole (78.8 g, 1.24 mol) were added. The mixture was shaken until all tetrazole was dissolved, N-methylimidazole (30 ml) was added, and mixture was left at room temperature for 5 hours. TEA (300 ml) was added, the mixture was diluted with DMF (1 L) and water (400 ml) and extracted with hexanes (3×3 L). The mixture was diluted with water (1.4 L) and extracted with the mixture of toluene (9 L) and hexanes (6 L). The two layers were separated and the upper layer was washed with DMF-water (60:40, v/v, 3×3 L) and water (3×2 L). The organic layer was dried (Na₂SO₄), filtered and evaporated to a sticky foam. The residue was co-evaporated with acetonitrile (2.5 L) under reduced pressure and dried in a vacuum oven (25° C., 0.1 mm Hg, 40 h) to afford 1350 g of an off-white foam solid (96%).

### Preparation of [5′-O-(4,4′-Dimethoxytriphenylmethyl)-2′-O-(2-methoxyethyl)-N⁴-isobutyrylguanosin-3′-O-yl]-2-cyanoethyl-N,N-diisopropylphosphoramidite G amidite)

5′-O-(4,4′-Dimethoxytriphenylmethyl)-2′-O-(2-methoxyethyl)-N⁴-isobutyrlguanosine (purchased from Reliable Biopharmaceutical, St. Louis, Mo., 1426 g, 2.0 mol) was dissolved in anhydrous DMF (2 L). The solution was co-evaporated with toluene (200 ml) at 50° C., cooled to room temperature and 2-cyanoethyl tetraisopropylphosphorodiamidite (900 g, 3.0 mol) and tetrazole (68 g, 0.97 mol) were added. The mixture was shaken until all tetrazole was dissolved, N-methylimidazole (30 ml) was added, and the mixture was left at room temperature for 5 hours. TEA (300 ml) was added, the mixture was diluted with DMF (2 L) and water (600 ml) and extracted with hexanes (3×3 L). The mixture was diluted with water (2 L) and extracted with a mixture of toluene (10 L) and hexanes (5 L). The two layers were separated and the upper layer was washed with DMF-water (60:40, v/v, 3×3 L). EtOAc (4 L) was added and the solution was washed with water (3×4 L). The organic layer was dried (Na₂SO₄), filtered and evaporated to approx. 4 kg. Hexane (4 L) was added, the mixture was shaken for 10 min, and the supernatant liquid was decanted. The residue was co-evaporated with acetonitrile (2×2 L) under reduced pressure and dried in a vacuum oven (25° C., 0.1 mm Hg, 40 h) to afford 1660 g of an off-white foamy solid (91%).

### 2′-O-(Aminooxyethyl) nucleoside amidites and 2′-O-(dimethylaminooxyethyl) nucleoside amidites

### 2′-(Dimethylaminooxyethoxy) nucleoside amidites

2′-(Dimethylaminooxyethoxy) nucleoside amidites (also known in the art as 2′-O-(dimethylaminooxyethyl) nucleoside amidites) are prepared as described in the following paragraphs. Adenosine, cytidine and guanosine nucleoside amidites are prepared similarly to the thymidine (5-methyluridine) except the exocyclic amines are protected with a benzoyl moiety in the case of adenosine and cytidine and with isobutyryl in the case of guanosine.

### 5′-O-tert-Butyldiphenylsilyl-O²-2′-anhydro-5-methyluridine

O²-2′-anhydro-5-methyluridine (Pro. Bio. Sint., Varese, Italy, 100.0 g, 0.416 mmol), dimethylaminopyridine (0.66 g, 0.013 eq, 0.0054 mmol) were dissolved in dry pyridine (500 ml) at ambient temperature under an argon atmosphere and with mechanical stirring. tert-Butyldiphenylchlorosilane (125.8 g, 119.0 mL, 1.1 eq, 0.458 mmol) was added in one portion. The reaction was stirred for 16 h at ambient temperature. TLC (R_{f} 0.22, EtOAc) indicated a complete reaction. The solution was concentrated under reduced pressure to a thick oil. This was partitioned between CH₂Cl₂ (1 L) and saturated sodium bicarbonate (2×1 L) and brine (1 L). The organic layer was dried over sodium sulfate, filtered, and concentrated under reduced pressure to a thick oil. The oil was dissolved in a 1:1 mixture of EtOAc and ethyl ether (600 mL) and cooling the solution to −10° C. afforded a white crystalline solid which was collected by filtration, washed with ethyl ether (3×2 00 mL) and dried (40° C., 1 mm Hg, 24 h) to afford 149 g of white solid (74.8%). TLC and NMR spectroscopy were consistent with pure product.

### 5′-O-tert-Butyldiphenylsilyl-2′-O-(2-hydroxyethyl)-5-methyluridine

In the fume hood, ethylene glycol (350 mL, excess) was added cautiously with manual stirring to a 2 L stainless steel pressure reactor containing borane in tetrahydrofuran (1.0 M, 2.0 eq, 622 mL). (Caution: evolves hydrogen gas). 5′-O-tert-Butyldiphenylsilyl-O²-2′-anhydro-5-methyluridine (149 g, 0.311 mol) and sodium bicarbonate (0.074 g, 0.003 eq) were added with manual stirring. The reactor was sealed and heated in an oil bath until an internal temperature of 160° C. was reached and then maintained for 16 h (pressure <100 psig). The reaction vessel was cooled to ambient temperature and opened. TLC (EtOAc, R_{f} 0.67 for desired product and R_{f} 0.82 for ara-T side product) indicated about 70% conversion to the product. The solution was concentrated under reduced pressure (10 to 1 mm Hg) in a warm water bath (40-100° C.) with the more extreme conditions used to remove the ethylene glycol. (Alternatively, once the THF has evaporated the solution can be diluted with water and the product extracted into EtOAc). The residue was purified by column chromatography (2 kg silica gel, EtOAc-hexanes gradient 1:1 to 4:1). The appropriate fractions were combined, evaporated and dried to afford 84 g of a white crisp foam (50%), contaminated starting material (17.4 g, 12% recovery) and pure reusable starting material (20 g, 13% recovery). TLC and NMR spectroscopy were consistent with 99% pure product.

### 2′-O-([2-phthalimidoxy)ethyl]-5′-t-butyldiphenylsilyl-5-methyluridine

5′-O-tert-Butyldiphenylsilyl-2′-O-(2-hydroxyethyl)-5-methyluridine (20 g, 36.98 mmol) was mixed with triphenylphosphine (11.63 g, 44.36 mmol) and N-hydroxyphthalimide (7.24 g, 44.36 mmol) and dried over P₂O₅ under high vacuum for two days at 40° C. The reaction mixture was flushed with argon and dissolved in dry THF (369.8 mL, Aldrich, sure seal bottle). Diethyl-azodicarboxylate (6.98 mL, 44.36 mmol) was added dropwise to the reaction mixture with the rate of addition maintained such that the resulting deep red coloration is just discharged before adding the next drop. The reaction mixture was stirred for 4 hrs., after which time TLC (EtOAc:hexane, 60:40) indicated that the reaction was complete. The solvent was evaporated in vacuuo and the residue purified by flash column chromatography (eluted with 60:40 EtOAc:hexane), to yield 2′-O-([2-phthalimidoxy)ethyl]-5′-t-butyldiphenylsilyl-5-methyluridine as white foam (21.819 g, 86%) upon rotary evaporation.

### 5′-O-tert-butyldiphenylsilyl-2′-O-[(2-formadoximinooxy)ethyl]-5-methyluridine

2′-O-([2-phthalimidoxy)ethyl]-5′-t-butyldiphenylsilyl-5-methyluridine (3.1 g, 4.5 mmol) was dissolved in dry CH₂Cl₂ (4.5 mL) and methylhydrazine (300 mL, 4.64 mmol) was added dropwise at −10° C. to 0° C. After 1 h the mixture was filtered, the filtrate washed with ice cold CH₂Cl₂, and the combined organic phase was washed with water and brine and dried (anhydrous Na₂SO₄). The solution was filtered and evaporated to afford 2′-O-(aminooxyethyl) thymidine, which was then dissolved in MeOH (67.5 mL). Formaldehyde (20% aqueous solution, w/w, 1.1 eq.) was added and the resulting mixture was stirred for 1 h. The solvent was removed under vacuum and the residue was purified by column chromatography to yield 5′-O-tert-butyldiphenylsilyl-2′-O-[(2-formadoximinooxy)ethyl]-5-methyluridine as white foam (1.95 g, 78%) upon rotary evaporation.

### 5′-O-tert-Butyldiphenylsilyl-2′-O—[N,N dimethylaminooxyethyl]-5-methyluridine

5′-O-tert-butyldiphenylsilyl-2′-O-[(2-formadoximinooxy)ethyl]-5-methyluridine (1.77 g, 3.12 mmol) was dissolved in a solution of 1M pyridinium p-toluenesulfonate (PPTS) in dry MeOH (30.6 mL) and cooled to 10° C. under inert atmosphere. Sodium cyanoborohydride (0.39 g, 6.13 mmol) was added and the reaction mixture was stirred. After 10 minutes the reaction was warmed to room temperature and stirred for 2 h. while the progress of the reaction was monitored by TLC (5% MeOH in CH₂Cl₂). Aqueous NaHCO₃ solution (5%, 10 mL) was added and the product was extracted with EtOAc (2×20 mL). The organic phase was dried over anhydrous Na₂SO₄, filtered, and evaporated to dryness. This entire procedure was repeated with the resulting residue, with the exception that formaldehyde (20% w/w, 30 mL, 3.37 mol) was added upon dissolution of the residue in the PPTS/MeOH solution. After the extraction and evaporation, the residue was purified by flash column chromatography and (eluted with 5% MeOH in CH₂Cl₂) to afford 5′-O-tert-butyldiphenylsilyl-2′-O—[N,N-dimethylaminooxyethyl]-5-methyluridine as a white foam (14.6 g, 80%) upon rotary evaporation.

### 2′-O-(dimethylaminooxyethyl)-5-methyluridine

Triethylamine trihydrofluoride (3.91 mL, 24.0 mmol) was dissolved in dry THF and TEA (1.67 mL, 12 mmol, dry, stored over KOH) and added to 5′-O-tert-butyldiphenylsilyl-2′-O-[N,N-dimethylaminooxyethyl]-5-methyluridine (1.40 g, 2.4 mmol). The reaction was stirred at room temperature for 24 hrs and monitored by TLC (5% MeOH in CH₂Cl₂). The solvent was removed under vacuum and the residue purified by flash column chromatography (eluted with 10% MeOH in CH₂Cl₂) to afford 2′-O-(dimethylaminooxyethyl)-5-methyluridine (766 mg, 92.5%) upon rotary evaporation of the solvent.

5′-O-DMT-2′-O-(dimethylaminooxyethyl)-5-methyluridine

2′-O-(dimethylaminooxyethyl)-5-methyluridine (750 mg, 2.17 mmol) was dried over P₂O₅ under high vacuum overnight at 40° C., co-evaporated with anhydrous pyridine (20 mL), and dissolved in pyridine (11 mL) under argon atmosphere. 4-dimethylaminopyridine (26.5 mg, 2.60 mmol) and 4,4′-dimethoxytrityl chloride (880 mg, 2.60 mmol) were added to the pyridine solution and the reaction mixture was stirred at room temperature until all of the starting material had reacted. Pyridine was removed under vacuum and the residue was purified by column chromatography (eluted with 10% MeOH in CH₂Cl₂ containing a few drops of pyridine) to yield 5′-O-DMT-2′-O-(dimethylamino-oxyethyl)-5-methyluridine (1.13 g, 80%) upon rotary evaporation.

### 5′-O-DMT-2′-O-(2-N,N-dimethylaminooxyethyl)-5-methyluridine-3′-[(2-cyanoethyl)-N,N-diisopropylphosphoramidite]

5′-O-DMT-2′-O-(dimethylaminooxyethyl)-5-methyluridine (1.08 g, 1.67 mmol) was co-evaporated with toluene (20 mL), N,N-diisopropylamine tetrazonide (0.29 g, 1.67 mmol) was added and the mixture was dried over P₂O₅ under high vacuum overnight at 40° C. This was dissolved in anhydrous acetonitrile (8.4 mL) and 2-cyanoethyl-N,N,N¹,N¹-tetraisopropylphosphoramidite (2.12 mL, 6.08 mmol) was added. The reaction mixture was stirred at ambient temperature for 4 h under inert atmosphere. The progress of the reaction was monitored by TLC (hexane:EtOAc 1:1). The solvent was evaporated, then the residue was dissolved in EtOAc (70 mL) and washed with 5% aqueous NaHCO₃ (40 mL). The EtOAc layer was dried over anhydrous Na₂SO₄, filtered, and concentrated. The residue obtained was purified by column chromatography (EtOAc as eluent) to afford 5′-O-DMT-2′-O-(2-N,N-dimethylaminooxyethyl)-5-methyluridine-3′-[(2-cyanoethyl)-N,N-diisopropylphosphoramidite] as a foam (1.04 g, 74.9%) upon rotary evaporation.

### 2′-(Aminooxyethoxy) nucleoside amidites

2′-(Aminooxyethoxy) nucleoside amidites (also known in the art as 2′-O-(aminooxyethyl) nucleoside amidites) are prepared as described in the following paragraphs. Adenosine, cytidine and thymidine nucleoside amidites are prepared similarly.

### N²-isobutyryl-6-O-diphenylcarbamoyl-2′-O-(2-ethylacetyl)-5′-O-(4,4′-dimethoxytrityl)guanosine-3′-[(2-cyanoethyl)-N,N-diisopropylphosphoramidite]

The 2′-O-aminooxyethyl guanosine analog may be obtained by selective 2′-O-alkylation of diaminopurine riboside. Multigram quantities of diaminopurine riboside may be purchased from Schering AG (Berlin) to provide 2′-O-(2-ethylacetyl) diaminopurine riboside along with a minor amount of the 3′-O-isomer. 2′-O-(2-ethylacetyl) diaminopurine riboside may be resolved and converted to 2′-O-(2-ethylacetyl)guanosine by treatment with adenosine deaminase. (McGee, D. P. C., Cook, P. D., Guinosso, C. J., WO 94/02501 A1 940203.) Standard protection procedures should afford 2′-O-(2-ethylacetyl)-5′-O-(4,4′-dimethoxytrityl)guanosine and 2-N-isobutyryl-6-O-diphenylcarbamoyl-2′-O-(2-ethylacetyl)-5′-O-(4,4′-dimethoxytrityl)guanosine which may be reduced to provide 2-N-isobutyryl-6-O-diphenylcarbamoyl-2′-O-(2-hydroxyethyl)-5′-O-(4,4′-dimethoxytrityl)guanosine. As before the hydroxyl group may be displaced by N-hydroxyphthalimide via a Mitsunobu reaction, and the protected nucleoside may be phosphitylated as usual to yield 2-N-isobutyryl-6-O-diphenylcarbamoyl-2′-O-([2-phthalmidoxy]ethyl)-5′-O-(4,4′-dimethoxytrityl)guanosine-3′-[(2-cyanoethyl)-N,N-diisopropylphosphoramidite].

### 2′-dimethylaminoethoxyethoxy (2′-DMAEOE) nucleoside amidites

2′-dimethylaminoethoxyethoxy nucleoside amidites (also known in the art as 2′-O-dimethylaminoethoxyethyl, i.e., 2′-O—CH₂—O—CH₂—N(CH₂)₂, or 2′-DMAEOE nucleoside amidites) are prepared as follows. Other nucleoside amidites are prepared similarly.

### 2′-O-[2(2-N,N-dimethylaminoethoxy)ethyl]-5-methyl uridine

2[2-(Dimethylamino)ethoxy]ethanol (Aldrich, 6.66 g, 50 mmol) was slowly added to a solution of borane in tetrahydrofuran (1 M, 10 mL, 10 mmol) with stirring in a 100 mL bomb. (Caution: Hydrogen gas evolves as the solid dissolves). O²-,2′-anhydro-5-methyluridine (1.2 g, 5 mmol), and sodium bicarbonate (2.5 mg) were added and the bomb was sealed, placed in an oil bath and heated to 155° C. for 26 h. then cooled to room temperature. The crude solution was concentrated, the residue was diluted with water (200 mL) and extracted with hexanes (200 mL). The product was extracted from the aqueous layer with EtOAc (3×200 mL) and the combined organic layers were washed once with water, dried over anhydrous sodium sulfate, filtered and concentrated. The residue was purified by silica gel column chromatography (eluted with 5:100:2 MeOH/CH₂Cl₂/TEA) as the eluent. The appropriate fractions were combined and evaporated to afford the product as a white solid.

### 5′-O-dimethoxytrityl-2′-O-[2(2-N,N-dimethyl-aminoethoxy)ethyl)]-5-methyl uridine

To 0.5 g (1.3 mmol) of 2′-O-[2(2-N,N-dimethylamino-ethoxy)ethyl)]-5-methyl uridine in anhydrous pyridine (8 mL), was added TEA (0.36 mL) and dimethoxytrityl chloride (DMT-Cl, 0.87 g, 2 eq.) and the reaction was stirred for 1 h. The reaction mixture was poured into water (200 mL) and extracted with CH₂Cl₂ (2×200 mL). The combined CH₂Cl₂ layers were washed with saturated NaHCO₃ solution, followed by saturated NaCl solution, dried over anhydrous sodium sulfate, filtered and evaporated. The residue was purified by silica gel column chromatography (eluted with 5:100:1 MeOH/CH₂Cl₂/TEA) to afford the product.

### 5′-O-Dimethoxytrityl-2′-O-[2(2-N,N-dimethylaminoethoxy)ethyl)]-5-methyl uridine-3′-O-(cyanoethyl-N,N-diisopropyl)phosphoramidite

Diisopropylaminotetrazolide (0.6 g) and 2-cyanoethoxy-N,N-diisopropyl phosphoramidite (1.1 mL, 2 eq.) were added to a solution of 5′-O-dimethoxytrityl-2′-O-[2(2-N,N-dimethylaminoethoxy)ethyl)]-5-methyluridine (2.17 g, 3 mmol) dissolved in CH₂Cl₂ (20 mL) under an atmosphere of argon. The reaction mixture was stirred overnight and the solvent evaporated. The resulting residue was purified by silica gel column chromatography with EtOAc as the eluent to afford the title compound.

### Example 2

### Oligonucleotide Synthesis

Unsubstituted and substituted phosphodiester (P═O) oligonucleotides are synthesized on an automated DNA synthesizer (Applied Biosystems model 380B) using standard phosphoramidite chemistry with oxidation by iodine.

Phosphorothioates (P═S) are synthesized as for the phosphodiester oligonucleotides except the standard oxidation bottle was replaced by 0.2 M solution of 3H-1,2-benzodithiole-3-one 1,1-dioxide in acetonitrile for the stepwise thiation of the phosphite linkages. The thiation wait step was increased to 68 sec and was followed by the capping step. After cleavage from the CPG column and deblocking in concentrated ammonium hydroxide at 55° C. (18 h), the oligonucleotides were purified by precipitating twice with 2.5 volumes of ethanol from a 0.5 M NaCl solution.

Phosphinate oligonucleotides are prepared as described in U.S. Pat. No. 5,508,270, herein incorporated by reference.

Alkyl phosphonate oligonucleotides are prepared as described in U.S. Pat. No. 4,469,863, herein incorporated by reference.

3′-Deoxy-3′-methylene phosphonate oligonucleotides are prepared as described in U.S. Pat. No. 5,610,289 or 5,625,050, herein incorporated by reference.

Phosphoramidite oligonucleotides are prepared as described in U.S. Pat. No. 5,256,775 or 5,366,878, herein incorporated by reference.

Alkylphosphonothioate oligonucleotides are prepared as described in published PCT applications PCT/US94/00902 and PCT/US93/06976 (published as WO 94/17093 and WO 94/02499, respectively), herein incorporated by reference. 3′-Deoxy-3′-amino phosphoramidate oligonucleotides are prepared as described in U.S. Pat. No. 5,476,925, herein incorporated by reference.

Phosphotriester oligonucleotides are prepared as described in U.S. Pat. No. 5,023,243, herein incorporated by reference.

Borano phosphate oligonucleotides are prepared as described in U.S. Pat. Nos. 5,130,302 and 5,177,198, both herein incorporated by reference.

### Example 3

### Oligonucleoside Synthesis

Methylenemethylimino linked oligonucleosides, also identified as MMI linked oligonucleosides, methylenedimethylhydrazo linked oligonucleosides, also identified as MDH linked oligonucleosides, and methylenecarbonylamino linked oligonucleosides, also identified as amide-3 linked oligonucleosides, and methyleneaminocarbonyl linked oligonucleosides, also identified as amide-4 linked oligonucleosides, as well as mixed backbone compounds having, for instance, alternating MMI and P═O or P═S linkages are prepared as described in U.S. Pat. Nos. 5,378,825, 5,386,023, 5,489,677, 5,602,240 and 5,610,289, all of which are herein incorporated by reference.

Formacetal and thioformacetal linked oligonucleosides are prepared as described in U.S. Pat. Nos. 5,264,562 and 5,264,564, herein incorporated by reference.

Ethylene oxide linked oligonucleosides are prepared as described in U.S. Pat. No. 5,223,618, herein incorporated by reference.

### Example 4

### PNA Synthesis

Peptide nucleic acids (PNAs) are prepared in accordance with any of the various procedures referred to in Peptide Nucleic Acids (PNA): Synthesis, Properties and Potential Applications, Bioorganic & Medicinal Chemistry, 1996, 4, 5-23. They may also be prepared in accordance with U.S. Pat. Nos. 5,539,082, 5,700,922, and 5,719,262, herein incorporated by reference.

### Example 5

### Oligonucleotide Isolation

After cleavage from the controlled pore glass column (Applied Biosystems) and deblocking in concentrated ammonium hydroxide at 55° C. for 18 hours, the oligonucleotides or oligonucleosides are purified by precipitation twice out of 0.5 M NaCl with 2.5 volumes ethanol. Synthesized oligonucleotides were analyzed by polyacrylamide gel electrophoresis on denaturing gels and judged to be at least 85% full length material. The relative amounts of phosphorothioate and phosphodiester linkages obtained in synthesis were periodically checked by ³¹P nuclear magnetic resonance spectroscopy, and for some studies oligonucleotides were purified by HPLC, as described by Chiang et al., J. Biol. Chem. 1991, 266, 18162-18171. Results obtained with HPLC-purified material were similar to those obtained with non-HPLC purified material.

### Example 6

### Oligonucleotide Synthesis—96 Well Plate Format

Oligonucleotides were synthesized via solid phase P(III) phosphoramidite chemistry on an automated synthesizer capable of assembling 96 sequences simultaneously in a standard 96 well format. Phosphodiester internucleotide linkages were afforded by oxidation with aqueous iodine. Phosphorothioate internucleotide linkages were generated by sulfurization utilizing 3,H-1,2 benzodithiole-3-one 1,1 dioxide (Beaucage Reagent) in anhydrous acetonitrile. Standard base-protected beta-cyanoethyldiisopropyl phosphoramidites were purchased from commercial vendors (e.g. PE-Applied Biosystems, Foster City, Calif., or Pharmacia, Piscataway, N.J.). Non-standard nucleosides are synthesized as per known literature or patented methods. They are utilized as base protected beta-cyanoethyldiisopropyl phosphoramidites.

Oligonucleotides were cleaved from support and deprotected with concentrated NH₄OH at elevated temperature (55-60° C.) for 12-16 hours and the released product then dried in vacuo. The dried product was then re-suspended in sterile water to afford a master plate from which all analytical and test plate samples are then diluted utilizing robotic pipettors.

### Example 7

### Oligonucleotide Analysis—96 Well Plate Format

The concentration of oligonucleotide in each well was assessed by dilution of samples and UV absorption spectroscopy. The full-length integrity of the individual products was evaluated by capillary electrophoresis (CE) in either the 96 well format (Beckman P/ACE™ MDQ) or, for individually prepared samples, on a commercial CE apparatus (e.g., Beckman P/ACE™ 5000, ABI 270). Base and backbone composition was confirmed by mass analysis of the compounds utilizing electrospray-mass spectroscopy. All assay test plates were diluted from the master plate using single and multi-channel robotic pipettors. Plates were judged to be acceptable if at least 85% of the compounds on the plate were at least 85% full length.

### Example 8

### Cell Culture and Oligonucleotide Treatment

The effect of antisense compounds on target nucleic acid expression can be tested in any of a variety of cell types provided that the target nucleic acid is present at measurable levels. Target RNA levels can be routinely determined using, for example, PCR or Northern blot analysis. The following 6 cell types are provided for illustrative purposes, but other cell types can be routinely used, provided that the target is expressed in the cell type chosen. This can be readily determined by methods routine in the art, for example Northern blot analysis, Ribonuclease protection assays, or RT-PCR.

### T-24 Cells:

The human transitional cell bladder carcinoma cell line T-24 was obtained from the American Type Culture Collection (ATCC) (Manassas, Va.). T-24 cells were routinely cultured in complete McCoy's 5A basal media (Gibco/Life Technologies, Gaithersburg, Md.) supplemented with 10% fetal calf serum (Gibco/Life Technologies, Gaithersburg, Md.), penicillin 100 units per mL, and streptomycin 100 micrograms per mL (Gibco/Life Technologies, Gaithersburg, Md.). Cells were routinely passaged by trypsinization and dilution when they reached 90% confluence. Cells were seeded into 96-well plates (Falcon-Primaria #3872) at a density of 7000 cells/well for use in RT-PCR analysis.

### A549 Cells:

The human lung carcinoma cell line A549 was obtained from the American Type Culture Collection (ATCC) (Manassas, Va.). A549 cells were routinely cultured in DMEM basal media (Gibco/Life Technologies, Gaithersburg, Md.) supplemented with 10% fetal calf serum (Gibco/Life Technologies, Gaithersburg, Md.), penicillin 100 units per mL, and streptomycin 100 micrograms per mL (Gibco/Life Technologies, Gaithersburg, Md.). Cells were routinely passaged by trypsinization and dilution when they reached 90% confluence.

### NHDF Cells:

Human neonatal dermal fibroblast (NHDF) were obtained from the Clonetics Corporation (Walkersville Md.). NHDFs were routinely maintained in Fibroblast Growth Medium (Clonetics Corporation, Walkersville Md.) supplemented as recommended by the supplier. Cells were maintained for up to 10 passages as recommended by the supplier.

### HEK Cells:

Human embryonic keratinocytes (HEK) were obtained from the Clonetics Corporation (Walkersville Md.). HEKs were routinely maintained in Keratinocyte Growth Medium (Clonetics Corporation, Walkersville Md.) formulated as recommended by the supplier. Cells were routinely maintained for up to 10 passages as recommended by the supplier.

### b.END Cells:

The mouse brain endothelial cell line b.END was obtained from Dr. Werner Risau at the Max Plank Instititute (Bad Nauheim, Germany). b.END cells are routinely cultured in DMEM, high glucose (Invitrogen Life Technologies, Carlsbad, Calif.) supplemented with 10% fetal bovine serum (Invitrogen Life Technologies, Carlsbad, Calif.). Cells are routinely passaged by trypsinization and dilution when they reach 90% confluence. Cells are seeded into 96-well plates (Falcon-Primaria #3872) at a density of 3000 cells/well for treatment with the oligomeric compounds of the invention.

### Primary Mouse Macrophages:

Macrophages were isolated as follows. Female C57Bl/6 mice (Charles River Laboratories, Wilmington, Mass.) were injected intraperitoneally with 1 ml 3% thioglycollate broth (Sigma-Aldrich, St. Louis, Mo.), and peritoneal macrophage cells were isolated by peritoneal lavage 4 days later. The cells were plated in 96-well plates at 40,000 cells/well for one hour in serum-free RPMI adjusted to contain 10 mM HEPES (Invitrogen Life Technologies, Carlsbad, Calif.), allowed to adhere, then non-adherent cells were washed away and the media was replaced with RPMI containing 10 mM HEPES, 10% FBS and penicillin/streptomycin (“complete” RPMI; Invitrogen Life Technologies, Carlsbad, Calif.).

Treatment with Antisense Compounds:

Cells are treated with oligonucleotide, generally when they reach 80% confluency. For cells grown in 96-well plates, wells are washed once with 200 μL OPTI-MEM™-1 reduced-serum medium (Gibco BRL) and then treated with 130 μL of OPTI-MEM™-1 containing 3.75 μg/mL LIPOFECTIN™ (Gibco BRL) and the desired concentration of oligonucleotide. After 4-7 hours of treatment, the medium is replaced with fresh medium. Cells are harvested 16-24 hours after oligonucleotide treatment.

For Northern blotting or other analysis, cells may be seeded onto 100 mm or other standard tissue culture plates and treated similarly, using appropriate volumes of medium and oligonucleotide.

### Example 9

### Analysis of Oligonucleotide Inhibition of Gene Expression

Antisense modulation of gene expression can be assayed in a variety of ways known in the art. For example, RNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+ mRNA. Methods of RNA isolation are taught in, for example, Ausubel, F. M. et al., Current Protocols in Molecular Biology, Volume 1, pp. 4.1.1-4.2.9 and 4.5.1-4.5.3, John Wiley & Sons, Inc., 1993. Northern blot analysis is routine in the art and is taught in, for example, Ausubel, F. M. et al., Current Protocols in Molecular Biology, Volume 1, pp. 4.2.1-4.2.9, John Wiley & Sons, Inc., 1996. Real-time quantitative (PCR) can be conveniently accomplished using the commercially available ABI PRISM™ 7700 Sequence Detection System, available from PE-Applied Biosystems, Foster City, Calif. and used according to manufacturer's instructions.

Protein levels can be quantitated in a variety of ways well known in the art, such as immunoprecipitation, Western blot analysis (immunoblotting), ELISA or fluorescence-activated cell sorting (FACS). Antibodies directed to the target protein can be identified and obtained from a variety of sources, such as the MSRS catalog of antibodies (Aerie Corporation, Birmingham, Mich.), or can be prepared via conventional antibody generation methods. Methods for preparation of polyclonal antisera are taught in, for example, Ausubel, F. M. et al., Current Protocols in Molecular Biology, Volume 2, pp. 11.12.1-11.12.9, John Wiley & Sons, Inc., 1997. Preparation of monoclonal antibodies is taught in, for example, Ausubel, F. M. et al., Current Protocols in Molecular Biology, Volume 2, pp. 11.4.1-11.11.5, John Wiley & Sons, Inc., 1997.

Immunoprecipitation methods are standard in the art and can be found at, for example, Ausubel, F. M. et al., Current Protocols in Molecular Biology, Volume 2, pp. 10.16.1-10.16.11, John Wiley & Sons, Inc., 1998. Western blot (immunoblot) analysis is standard in the art and can be found at, for example, Ausubel, F. M. et al., Current Protocols in Molecular Biology, Volume 2, pp. 10.8.1-10.8.21, John Wiley & Sons, Inc., 1997. Enzyme-linked immunosorbent assays (ELISA) are standard in the art and can be found at, for example, Ausubel, F. M. et al., Current Protocols in Molecular Biology, Volume 2, pp. 11.2.1-11.2.22, John Wiley & Sons, Inc., 1991.

### Example 10

Poly(A)+ mRNA Isolation

Poly(A)+ mRNA is isolated according to Miura et al., Clin. Chem., 1996, 42, 1758-1764. Other methods for poly(A)+ mRNA isolation are taught in, for example, Ausubel, F. M. et al., Current Protocols in Molecular Biology, Volume 1, pp. 4.5.1-4.5.3, John Wiley & Sons, Inc., 1993. Briefly, for cells grown on 96-well plates, growth medium is removed from the cells and each well is washed with 200 μL cold PBS. 60 μL lysis buffer (10 mM Tris-HCl, pH 7.6, 1 mM EDTA, 0.5 M NaCl, 0.5% NP-40, 20 mM vanadyl-ribonucleoside complex) was added to each well, the plate is gently agitated and then incubated at room temperature for five minutes. 55 μL of lysate is transferred to Oligo d(T) coated 96-well plates (AGCT Inc., Irvine Calif.). Plates are incubated for 60 minutes at room temperature, washed 3 times with 200 μL of wash buffer (10 mM Tris-HCl pH 7.6, 1 mM EDTA, 0.3 M NaCl). After the final wash, the plate is blotted on paper towels to remove excess wash buffer and then air-dried for 5 minutes. 60 μL of elution buffer (5 mM Tris-HCl pH 7.6), preheated to 70° C. is added to each well, the plate is incubated on a 90° C. hot plate for 5 minutes, and the eluate is then transferred to a fresh 96-well plate.

Cells grown on 100 mm or other standard plates may be treated similarly, using appropriate volumes of all solutions.

### Example 11

### Total RNA Isolation

Total RNA is isolated using an RNEASY 96™ kit and buffers purchased from Qiagen Inc. (Valencia Calif.) following the manufacturer's recommended procedures. Briefly, for cells grown on 96-well plates, growth medium is removed from the cells and each well is washed with 200 μL cold PBS. 100 μL Buffer RLT is added to each well and the plate vigorously agitated for 20 seconds. 100 μL of 70% ethanol is then added to each well and the contents mixed by pipetting three times up and down. The samples are then transferred to the RNEASY 96™ well plate attached to a QIAVAC™ manifold fitted with a waste collection tray and attached to a vacuum source. Vacuum is applied for 15 seconds. 1 mL of Buffer RW1 is added to each well of the RNEASY 96™ plate and the vacuum again applied for 15 seconds. 1 mL of Buffer RPE is then added to each well of the RNEASY 96™ plate and the vacuum applied for a period of 15 seconds. The Buffer RPE wash is then repeated and the vacuum is applied for an additional 10 minutes. The plate is then removed from the QIAVAC™ manifold and blotted dry on paper towels. The plate is then re-attached to the QIAVAC™ manifold fitted with a collection tube rack containing 1.2 mL collection tubes. RNA is then eluted by pipetting 60 μL water into each well, incubating 1 minute, and then applying the vacuum for 30 seconds. The elution step is repeated with an additional 60 μl water.

The repetitive pipetting and elution steps may be automated using a QIAGEN Bio-Robot 9604 (Qiagen, Inc., Valencia Calif.). Essentially, after lysing of the cells on the culture plate, the plate is transferred to the robot deck where the pipetting, DNase treatment and elution steps are carried out.

### Example 12

### Real-Time Quantitative PCR Analysis of Target mRNA Levels

Quantitation of target mRNA levels is accomplished by real-time quantitative PCR using the ABI PRISM™ 7700 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR, in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., JOE, FAM, or VIC, obtained from either Operon Technologies Inc., Alameda, Calif. or PE-Applied Biosystems, Foster City, Calif.) is attached to the 5′ end of the probe and a quencher dye (e.g., TAMRA, obtained from either Operon Technologies Inc., Alameda, Calif. or PE-Applied Biosystems, Foster City, Calif.) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM™ 7700 Sequence Detection System. In each assay, a series of parallel reactions containing serial dilutions of mRNA from untreated control samples generates a standard curve that is used to quantitate the percent inhibition after antisense oligonucleotide treatment of test samples.

Prior to quantitative PCR analysis, primer-probe sets specific to the target gene being measured may be evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

PCR reagents are obtained from PE-Applied Biosystems, Foster City, Calif. RT-PCR reactions were carried out by adding 25 μL PCR cocktail (1× TAQMAN™ buffer A, 5.5 mM MgCl₂, 300 μM each of dATP, dCTP and dGTP, 600 μM of dUTP, 100 nM each of forward primer, reverse primer, and probe, 20 Units RNAse inhibitor, 1.25 Units AMPLITAQ GOLD™, and 12.5 Units MuLV reverse transcriptase) to 96 well plates containing 25 μL total RNA solution. The RT reaction is carried out by incubation for 30 minutes at 48° C. Following a 10 minute incubation at 95° C. to activate the AMPLITAQ GOLD™, 40 cycles of a two-step PCR protocol are carried out: 95° C. for 15 seconds (denaturation) followed by 60° C. for 1.5 minutes (annealing/extension).

Gene target quantities obtained by real time RT-PCR are normalized using either the expression level of GAPDH, a gene whose expression is constant, or by quantifying total RNA using RiboGreen™ (Molecular Probes, Inc. Eugene, Oreg.). GAPDH expression is quantified by real time RT-PCR, by being run simultaneously with the target, multiplexing, or separately. Total RNA is quantified using RiboGreen™ RNA quantification reagent from Molecular Probes. Methods of RNA quantification by RiboGreen™ are taught in Jones, L. J., et al, Analytical Biochemistry, 1998, 265, 368-374.

In this assay, 175 μL of RiboGreen™ working reagent (RiboGreen™ reagent diluted 1:2865 in 10 mM Tris-HCl, 1 mM EDTA, pH 7.5) is pipetted into a 96-well plate containing 25 uL purified, cellular RNA. The plate is read in a CytoFluor 4000 (PE Applied Biosystems) with excitation at 480 nm and emission at 520 nm.

### Example 13

### Northern Blot Analysis of Target mRNA Levels

Eighteen hours after antisense treatment, cell monolayers are washed twice with cold PBS and lysed in 1 mL RNAZOL™ (TEL-TEST “B” Inc., Friendswood, Tex.). Total RNA is prepared following manufacturer's recommended protocols. Twenty micrograms of total RNA is fractionated by electrophoresis through 1.2% agarose gels containing 1.1% formaldehyde using a MOPS buffer system (AMRESCO, Inc. Solon, Ohio). RNA is transferred from the gel to HYBOND™-N+ nylon membranes (Amersham Pharmacia Biotech, Piscataway, N.J.) by overnight capillary transfer using a Northern/Southern Transfer buffer system (TEL-TEST “B” Inc., Friendswood, Tex.). RNA transfer is confirmed by UV visualization. Membranes are fixed by UV cross-linking using a STRATALINKER™ UV Crosslinker 2400 (Stratagene, Inc, La Jolla, Calif.) and then probed using QUICKHYB™ hybridization solution (Stratagene, La Jolla, Calif.) using manufacturer's recommendations for stringent conditions.

Hybridized membranes are visualized and quantitated using a PHOSPHORIMAGER™ and IMAGEQUANT™ Software V3.3 (Molecular Dynamics, Sunnyvale, Calif.). Data are normalized to GAPDH levels in untreated controls.

### Example 14

### Reduction of Human C-Raf mRNA Levels by Treatment with Uniformly 2′-MOE Modified Phosphorothioate Antisense Oligonucleotides Targeted to mRNA Splice Sites

In accordance with the present invention, a series of oligonucleotides were designed to target different regions of the human c-raf RNA, using published sequences. The oligonucleotides are shown in Table 1. “Target site” indicates the first (5′-most) nucleotide number on the particular target sequence to which the oligonucleotide binds. The human c-raf target sequence (provided herein as SEQ ID NO: 1) is a concatenation of human c-raf genomic sequence contigs from Genbank accession numbers AC026153.10 and AC018500.2. All compounds in Table 1 except as indicated are uniformly modified, i.e., composed of 2′-methoxyethyl (2′-MOE) nucleotides at each position. The internucleoside (backbone) linkages are phosphorothioate (P═S) throughout the oligonucleotide. All cytidine residues are 5-methylcytidines. The compounds were analyzed for their effect on c-raf mRNA levels in T24 cells. LIPOFECTIN/OptiMEM mixture was prepared by mixing 185 ml OptiMEM and 2.22 ml LIPOFECTIN and vortexing for 15 minutes at room temperature. 6 ml LIPOFECTIN/OptiMEM was aliquotted into 15 ml tubes and oligonucleotide was added to give 400 nM oligonucleotide. The mixture was vortexed for 15 minutes at room temperature. T24 cells were washed in PBS and oligonucleotide mixture was added (200 μl/well for 96 well plated, 5 ml/dish if done in 10 cm dishes). Cells were incubated for 4 hours at 37° C., 5% CO₂. Oligonucleotide mixture was aspirated and replaced with growth medium (GM) with 1% fetal calf serum. Cells were incubated at 37° C., 5% CO₂ overnight. Plates were washed 1× with PBS and RNA was isolated by the Qiagen RNEASY protocol. Quantitative RT-PCR was carried out as described in other examples herein. Data are shown as percent of untreated control and are averages from multiple experiments. If present, “N.D.” indicates “no data”.

TABLE 1
Reduction of human c-raf mRNA levels by uniformly modified
2′-MOE phosphorothioate oligonucleotides

| | | | | | % reduction | |
|---|---|---|---|---|---|---|
| | | TARGET | TARGET | | in mRNA | |
| ISIS # | REGION | SEQ ID NO | SITE | SEQUENCE | levels | SEQ ID NO. |
| 154127 | Transcription | 1 | 8345 | GGTGCTCGTCCTCCCGACCT | 0 | 2 |
| | start site | | | | | |
| 154128 | Exon 1/Intron 1 | 1 | 8699 | TGCCACCTACCTGAGGGAGC | 0 | 3 |
| | junction | | | | | |
| 154129 | Intron 1/Exon 2 | 1 | 20510 | ATTCTTAAACCTGGTAAGAA | 8 | 4 |
| | junction | | | | | |
| 154130 | Exon 2/Intron 2 | 1 | 20743 | GTTCACATACCACTGTTCTT | 0 | 5 |
| | junction | | | | | |
| 154131 | Intron 2/Exon 3 | 1 | 27195 | GCACATTGACCTACAAACAA | 0 | 6 |
| | junction | | | | | |
| 154132 | Exon 3/Intron 3 | 1 | 27308 | GAGCTCTTACCCTTTGTGTT | 2 | 7 |
| | junction | | | | | |
| 154133 | Exon 4/Intron 4 | 1 | 30025 | TGCAACTTACAAAGTTGTGT | 18 | 8 |
| | junction | | | | | |
| 154134 | Intron 4/Exon 5 | 1 | 30334 | TCTTCCGAGCCTACAACAAG | 0 | 9 |
| | junction | | | | | |
| 154135 | Exon 5/Intron 5 | 1 | 30492 | AATGCCTTACAAGAGTTGTC | 0 | 10 |
| | junction | | | | | |
| 154136 | Intron 6/Exon 7 | 1 | 34981 | GTGCTGAGAACTAGGAGGAG | 4 | 11 |
| | junction | | | | | |
| 154137 | Exon 7/Intron 7 | 1 | 35135 | GCCCTATTACCTCAATCATC | 0 | 12 |
| | junction | | | | | |
| 154138 | Intron 7/Exon 8 | 1 | 38855 | GAATTGCATCCTGAAACAGA | 26 | 13 |
| | junction | | | | | |
| 154139 | Exon 8/Intron 8 | 1 | 38883 | GGAAAAGTACCTGATTCGCT | 61 | 14 |
| | junction | | | | | |
| 154140 | Intron 8/exon 9 | 1 | 38991 | GAAGGTGAGGCTTAATAGAC | 19 | 15 |
| | junction | | | | | |
| 154141 | Intron 9/Exon | 1 | 39462 | CACGAGGCCTCTGAAACAAG | 0 | 16 |
| | 10 junction | | | | | |
| 154142 | Exon 10/Intron | 1 | 39580 | CCAAGCTTACCGTGCCATTT | 59 | 17 |
| | 10 junction | | | | | |
| 154143 | Intron 10/Exon | 1 | 47482 | GCAACATCTCCTGCAAAATT | 0 | 18 |
| | 11 junction | | | | | |
| 154144 | Exon 11/Intron | 1 | 47567 | TTCTACTCACCGCAGAACAG | 0 | 19 |
| | 11 junction | | | | | |
| 154145 | Intron 12/Exon | 1 | 51633 | ATGCAAATAGCTGTGAAGGG | 0 | 20 |
| | 13 junction | | | | | |
| 154146 | Exon 13/Intron | 1 | 51680 | CAAAGGATACTGTTGGATTT | 71 | 21 |
| | 13 junction | | | | | |
| 154147 | Intron 13/Exon | 1 | 53471 | AGAAATATATCTCAATGCTT | 0 | 22 |
| | 14 junction | | | | | |
| 154148 | Exon 14/Intron | 1 | 53590 | AGATTCTCACCATCCAGAGG | 0 | 23 |
| | 14 junction | | | | | |
| 154149 | Exon 15/Intron | 1 | 54149 | ACAGACTTACCTGATCTCGG | 0 | 24 |
| | 15 junction | | | | | |
| 154150 | Intron 15/Exon | 1 | 54289 | TGAAGATGATCTAAGGGAAA | 0 | 25 |
| | 16 junction | | | | | |
| 13650 | c-raf 3′ UTR | 1 | 55175 | TCCCGCCTGTGACATGCATT | 75 | 26 |
| | MOE gapmer | | | 2′ MOE at positions | | |
| | | | | 1-6 and 15-20, 2′ | | |
| | | | | deoxy at positions 7- | | |
| | | | | 14 | | |
| 147979 | c-raf 3′ UTR | 1 | 55175 | TCCCGCCTGTGACATGCATT | 79 | 26 |
| | MOE gapmer | | | 2′ MOE at positions | | |
| | | | | 1-6 and 15-20, 2′ | | |
| | | | | deoxy at positions 7- | | |
| | | | | 14; FITC label | | |

ISIS 13650 and 147979 are chimeric oligonucleotides (“gapmers”) 20 nucleotides in length targeted to human c-raf, composed of a central “gap” region consisting of ten 2′-deoxynucleotides, which is flanked on both sides (5′ and 3′ directions) by five-nucleotide “wings”. The wings are composed of 2′-methoxyethyl (2′-MOE) nucleotides. The internucleoside (backbone) linkages are phosphorothioate (P═S) throughout the oligonucleotide.

As shown in Table 1, it was surprisingly found that a number of uniformly modified oligonucleotides caused reduction of c-raf target RNA levels. ISIS 154139, 154142 and 154146 (SEQ ID NO: 14, 17 and 21) demonstrated at least 50% reduction of human c-raf RNA levels in this assay and are therefore preferred. These oligonucleotides are believed to be unable to elicit RNAse H cleavage of the target mRNA.

### Example 15

### Analysis of C-Raf Protein Levels

Cells were treated with oligonucleotides as described in the previous example, then after oligonucleotide was replaced with growth medium, cells were incubated at 37° C., 5% CO₂ for 48 hours. The GM was transferred to a 15 ml conical tube. Plates were washed with PBS. 5 ml PBS was transferred to the tube with GM, centrifuged at 1500 rpm for 10 minutes, and cell lysate from dish was added to pellet. 0.25 ml RIPA lysis buffer (1% NP-40, 0.5% Na deoxycholate, 0.1% SDS in PBS) with inhibitors was added, and cells were scraped and the resulting lysate was added to above cell pellet. Lysate was transferred to a 1.5 ml Eppendorf tube and centrifuged at 14,000 rpm for 15 minutes at 4° C. The supernatant was transferred to new Eppendorf tubes and total protein was quantitated using the BioRad (Hercules Calif.) DC Protein assay.

Western blot analysis (immunoblot analysis) of c-raf protein levels was carried out using standard methods. Cells are harvested, suspended in Laemmli buffer (100 μl/well), boiled for 5 minutes and loaded on a 10% SDS-PAGE gel. Gels are run for 1.5 hours at 150 V, and transferred to membrane (2 hr, 50V) for western blotting. Appropriate primary antibody directed to the target protein is used, with a radiolabelled or fluorescently labeled secondary antibody directed against the primary antibody species. Bands are visualized using a PHOSPHORIMAGER™ (Molecular Dynamics, Sunnyvale Calif.). Results are shown in Table 2, expressed as percent of control.

TABLE 2
Reduction of human c-raf protein levels by uniformly
modified 2′-MOE phosphorothioate oligonucleotides

| | | % reduction in | SEQ ID |
|---|---|---|---|
| ISIS # | REGION | protein | NO |
| 154127 | Transcription start | 14 | 2 |
| | site | | |
| 154128 | Exon 1/Intron 1 | 23 | 3 |
| | junction | | |
| 154129 | Intron 1/Exon 2 | 8 | 4 |
| | junction | | |
| 154130 | Exon 2/Intron 2 | 7 | 5 |
| | junction | | |
| 154131 | Intron 2/Exon 3 | 45 | 6 |
| | junction | | |
| 154132 | Exon 3/Intron 3 | 72 | 7 |
| | junction | | |
| 154133 | Exon 4/Intron 4 | 31 | 8 |
| | junction | | |
| 154134 | Intron 4/Exon 5 | 0 | 9 |
| | junction | | |
| 154135 | Exon 5/Intron 5 | 0 | 10 |
| | junction | | |
| 154136 | Intron 6/Exon 7 | 37 | 11 |
| | junction | | |
| 154137 | Exon 7/Intron 7 | 13 | 12 |
| | junction | | |
| 154138 | Intron 7/Exon 8 | 54 | 13 |
| | junction | | |
| 154139 | Exon 8/Intron 8 | 95 | 14 |
| | junction | | |
| 154140 | Intron 8/exon 9 | 48 | 15 |
| | junction | | |
| 154141 | Intron 9/Exon 10 | 0 | 16 |
| | junction | | |
| 154142 | Exon 10/Intron 10 | 73 | 17 |
| | junction | | |
| 154143 | Intron 10/Exon 11 | 11 | 18 |
| | junction | | |
| 154144 | Exon 11/Intron 11 | 39 | 19 |
| | junction | | |
| 154145 | Intron 12/Exon 13 | 31 | 20 |
| | junction | | |
| 154146 | Exon 13/Intron 13 | 69 | 21 |
| | junction | | |
| 154147 | Intron 13/Exon 14 | 35 | 22 |
| | junction | | |
| 154148 | Exon 14/Intron 14 | 46 | 23 |
| | junction | | |
| 154149 | Exon 15/Intron 15 | 52 | 24 |
| | junction | | |
| 154150 | Intron 15/Exon 16 | 16 | 25 |
| | junction | | |
| 13650 | c-raf 3′ UTR MOE | 64 | 26 |
| | gapmer | | |
| 147979 | c-raf 3′ UTR MOE | 58 | 26 |
| | gapmer; FITC | | |

From Table 2 it can be observed that antisense compounds which caused RNA reduction (Table 1) also caused reduction in the corresponding protein.

### Example 16

### Reduction of C-Raf mRNA and Protein Levels is Dose-Dependent

ISIS 154142 (SEQ ID NO: 17) was tested at various doses to determine whether the reduction it caused in c-raf RNA and protein levels was dose-dependent. For comparison, ISIS 154132 (SEQ ID NO: 7), which did not show reduction of target RNA levels, was also tested. Oligonucleotide treatment of T24 cells was as described in previous examples, using oligonucleotide concentrations of 0, 25, 100 and 400 nM. ISIS 154132 did not show a dose-dependent reduction in c-raf mRNA (reductions of approximately 0, 22%, 2 and 21% at concentrations of 0, 25, 100 and 400 nM, respectively) though reduction of c-raf protein by this oligonucleotide was dose-dependent (protein reduction at 0, 25, 100 and 400 nM oligo treatment was approximately 0, 21, 74 and 82%. In contrast, ISIS 154142 showed a dose-dependent inhibition of both RNA and protein. For mRNA, reduction at 0, 25, 100 and 400 nM oligo treatment was approximately 0, 49, 75 and 69%. For protein, reduction at 0, 25, 100 and 400 nM oligo treatment was approximately 0, 35, 67 and 76%.

### Example 17

### Reduction of Human JNK1 mRNA Levels by Treatment with Uniformly 2′-MOE Modified Phosphorothioate Antisense Oligonucleotides

In accordance with the present invention, a series of oligonucleotides were designed to target different regions of the human JNK1 RNA, using published sequences (residues 48001-84000 from Genbank accession no. AC016397.5, which are provided herein as SEQ ID NO. 27. The oligonucleotides are shown in Table 3. “Target site” indicates the first (5′-most) nucleotide number on the particular target sequence to which the oligonucleotide binds. All compounds in Table 3 except as indicated are uniformly modified, i.e., composed of 2′-methoxyethyl (2′-MOE) nucleotides at each position. The internucleoside (backbone) linkages are phosphorothioate (P═S) throughout the oligonucleotide. All cytidine residues are 5-methylcytidines. The compounds were analyzed for their effect on JNK mRNA and protein levels in A549 cells by quantitative real-time PCR as described in other examples herein. Oligonucleotide treatment was as described in Example 14 above. Data are shown as percent of untreated control and are averages from multiple experiments. If present, “N.D.” indicates “no data”.

TABLE 3
Reduction of human JNK1 mRNA levels in A549 cells by uniformly
modified 2′-MOE phosphorothioate oligonucleotides

| | | TARGET | TARGET | | % reduction | SEQ ID |
|---|---|---|---|---|---|---|
| ISIS # | REGION | SEQ ID NO | SITE | SEQUENCE | in mRNA | NO. |
| 154151 | Intron 1/Exon 2 | 27 | 4640 | ATAAGCTGCGCTGTAATAAG | 0 | 28 |
| | junction | | | | | |
| 154152 | Intron 2/Exon 3 | 27 | 9667 | GGCCAATTATCTATAATAAA | 11 | 29 |
| | junction | | | | | |
| 154153 | Exon 3/Intron 3 | 27 | 9726 | TTACACTTACACATCTTGAA | 16 | 30 |
| | junction | | | | | |
| 154154 | Intron 3/Exon 4 | 27 | 9818 | GACTATGTAACTTTATGAGT | 28 | 31 |
| | junction | | | | | |
| 154155 | Exon 4/Intron 4 | 27 | 9957 | TTCTACTAACCCGATGAATA | 49 | 32 |
| | junction | | | | | |
| 154156 | Intron 4/Exon 5 | 27 | 19943 | GCTTTAAGTCCTTCAGAAAA | 53 | 33 |
| | junction | | | | | |
| 154157 | Exon 5/Intron 5 | 27 | 20109 | GTGTGCTGACCGTTTTCCTT | 38 | 34 |
| | junction | | | | | |
| 154158 | Intron 5/Exon 6 | 27 | 23876 | CATAAATCCACTATATGTTT | 0 | 35 |
| | junction | | | | | |
| 154159 | Exon 6/Intron 6 | 27 | 23948 | ACAAGGATACAGTCCCTTCC | 0 | 36 |
| | junction | | | | | |
| 154160 | Intron 6/Exon 7 | 27 | 25676 | TGATCAATATCTAATATCAA | 0 | 37 |
| | junction | | | | | |
| 154161 | Exon 7/ Intron 7 | 27 | 25859 | TAAAAAGTACCTTTAAGTTT | 2 | 38 |
| | junction | | | | | |
| 154162 | Intron 7/Exon 8 | 27 | 26168 | GCCTGACTGGCTGCAAACAT | 5 | 39 |
| | junction | | | | | |
| 154163 | Exon 8/Intron 8 | 27 | 26293 | AATAACTTACAGCTTCTGCT | 3 | 40 |
| | junction | | | | | |
| 154164 | Intron 8/Exon 9 | 27 | 26868 | TTGGTGGTGGCTGAAAAACA | 30 | 41 |
| | junction | | | | | |
| 154165 | Exon 9/Intron 9 | 27 | 26932 | ACGAATGTACCTTTCCACTC | 59 | 42 |
| | junction | | | | | |
| 154166 | Intron 9/Exon 10 | 27 | 30981 | TATATCAATTCTGTAAAAGA | 1 | 43 |
| | junction | | | | | |
| 154167 | Exon 10/Intron 10 | 27 | 31059 | TGTAACCAACCTAAAGGAGA | 0 | 44 |
| | junction | | | | | |
| 154168 | Intron 10/Exon 11 | 27 | 34667 | TGCACCTGTGCTATGAGAAA | 0 | 45 |
| | junction | | | | | |
| 15346 | Coding region | 27 | 218 | CTCTCTGTAGGCCCGCTTGG | 92 | 46 |
| | | | | JNK1 MOE Gapmer | | |
| 18076 | Scrambled control | | | CTTTCCGTTGGACCCCTGGG | 8 | 47 |
| | for 15346 | | | Scrambled MOE Gapmer | | |

ISIS 15346 and 18076 are chimeric oligonucleotides (“gapmers”) 20 nucleotides in length targeted to human JNK1, composed of a central “gap” region consisting of ten 2′-deoxynucleotides, which is flanked on both sides (5′ and 3′ directions) by five-nucleotide “wings”. The wings are composed of 2′-methoxyethyl (2′-MOE) nucleotides. The internucleoside (backbone) linkages are phosphorothioate (P═S) throughout the oligonucleotide.

As shown in Table 3, it was surprisingly found that several uniform 2′MOE antisense oligonucleotides were able to reduce target RNA levels. Of these, ISIS 145155, 154156 and 154165 (SEQ ID NO; 32, 33 and 42) demonstrated at least 40% reduction of human JNK1 RNA levels in this assay and are preferred. Oligonucleotides with these modifications have been demonstrated to be unable to elicit RNAse H cleavage of their complementary target mRNA.

### Example 18

### Analysis of Human JNK1 Protein Levels

Western blot analysis (immunoblot analysis) of JNK1 protein levels was carried out using standard methods as described in previous examples. Results are shown in Table 4, expressed as percent of control.

TABLE 4
Reduction of human JNK1 protein levels by uniformly
modified 2′-MOE phosphorothioate oligonucleotides

| | | % reduction in | SEQ ID |
|---|---|---|---|
| ISIS # | REGION | JNK1 protein | NO |
| 154151 | Intron 1/Exon 2 | 22 | 28 |
| | junction | | |
| 154152 | Intron 2/Exon 3 | 47 | 29 |
| | junction | | |
| 154153 | Exon 3/Intron 3 | 35 | 30 |
| | junction | | |
| 154154 | Intron 3/Exon 4 | 33 | 31 |
| | junction | | |
| 154155 | Exon 4/Intron 4 | 51 | 32 |
| | junction | | |
| 154156 | Intron 4/Exon 5 | 61 | 33 |
| | junction | | |
| 154157 | Exon 5/Intron 5 | 60 | 34 |
| | junction | | |
| 154158 | Intron 5/Exon 6 | 0 | 35 |
| | junction | | |
| 154159 | Exon 6/Intron 6 | 0 | 36 |
| | junction | | |
| 154160 | Intron 6/Exon 7 | 3 | 37 |
| | junction | | |
| 154161 | Exon 7/Intron 7 | 51 | 38 |
| | junction | | |
| 154162 | Intron 7/Exon 8 | 21 | 39 |
| | junction | | |
| 154163 | Exon 8/Intron 8 | 35 | 40 |
| | junction | | |
| 154164 | Intron 8/Exon 9 | 30 | 41 |
| | junction | | |
| 154165 | Exon 9/Intron 9 | 72 | 42 |
| | junction | | |
| 154166 | Intron 9/Exon 10 | 46 | 43 |
| | junction | | |
| 154167 | Exon 10/Intron 10 | 70 | 44 |
| | junction | | |
| 154168 | Intron 10/Exon 11 | 26 | 45 |
| | junction | | |
| 15346 | Coding region | 60 | 46 |
| 18076 | Scrambled control | 16 | 47 |
| | for 15346 | | |

From Table 4 it can be observed that antisense compounds which caused JNK1 mRNA reduction (Table 3) also caused reduction in the corresponding JNK1 protein.

### Example 19

### Reduction of Rat Collapsin Response Mediator Protein 2 (CRMP-2) mRNA Levels by Treatment with Uniformly 2′-MOE Modified Phosphorothioate Antisense Oligonucleotides Targeted to CRMP-2 mRNA Splice Sites

In accordance with the present invention, a series of oligonucleotides were designed to target different regions of the rat collapsin response mediator protein 2 (CRMP-2) RNA, using published sequences. Genbank accession no. 246882.1 is provided herein as SEQ ID NO: 48. Partial genomic sequence for exons 1-14 with two nucleotides of flanking intron sequences (on one or both ends) are provided herein as SEQ ID NO: 49-62. The oligonucleotides are shown in Table 5 as SEQ ID NO: 63-97. “Target site” indicates the first (5′-most) nucleotide number on the particular target sequence to which the oligonucleotide binds. All compounds in Table 5 except as indicated are uniformly modified, having a 2′-MOE nucleotide at each position. The internucleoside linkages are phosphorothioate (P═S) throughout the oligonucleotide. All cytidine residues are 5-methylcytidines. The compounds were analyzed for their effect on CRMP-2 mRNA levels in PC-12 cells (American Type Culture Collection, Manassas Va.) by quantitative real-time PCR as described in other examples herein. Data are shown as percent of untreated control and are averages from multiple experiments. If present, “N.D.” indicates “no data”.

TABLE 5
Inhibition of rat collapsin response mediator protein 2 mRNA levels
by uniformly modified 2′-MOE phosphorothioate oligonucleotides

| | TARGET | | | | % | |
|---|---|---|---|---|---|---|
| | SEQ ID | TARGET | | | decrease | SEQ |
| ISIS # | NO | SITE | REGION | SEQUENCE | in RNA | ID NO |
| 155057 | 48 | 1 | 5′ UTR | AAGAGACAGATGCAATCCTC | 0 | 63 |
| 155058 | 48 | 33 | 5′ UTR | CTGGTCTTGCTATTAGGAGA | 0 | 64 |
| 155059 | 48 | 42 | 5′ UTR | ATCCCTTAGCTGGTCTTGCT | 0 | 65 |
| 155060 | 48 | 63 | 5′ UTR | TATTTGTAGGAAAAAGGTAC | 0 | 66 |
| 155061 | 48 | 89 | 5′ UTR | CTTGGTTTAAAATATATATA | 12 | 67 |
| 155062 | 48 | 117 | 5′ UTR | TTAAAGCAAAGAGAGCCGGA | 4 | 68 |
| 155063 | 48 | 141 | 5′ UTR | GGAAGTAATTTCAAGAGGAC | 0 | 69 |
| 155064 | 48 | 170 | Start codon | CTGATAAGACATCTCTCCGG | 0 | 70 |
| 155065 | 48 | 2888 | PolyA signal | TTGGTGACTTAATCAGGACC | 0 | 71 |
| 155066 | 49 | 199 | Exon 1/Intron 1 | ACCGTGATGCGTGGAATATT | 6 | 72 |
| | | | junction | | | |
| 155067 | 50 | 1 | Intron 1/ Exon 2 | GATCAGAAGACGATCGCTCT | 4 | 73 |
| | | | junction | | | |
| 155068 | 50 | 74 | Exon 2/Intron 2 | ACTTGATCAACCCATCTTCC | 0 | 74 |
| | | | junction | | | |
| 155069 | 51 | 1 | Intron 2/Exon 3 | AGGTTTTCTCCTATTTGCCT | 0 | 75 |
| | | | junction | | | |
| 155070 | 51 | 170 | Exon 3/Intron 3 | ACTGATCATGGTGGTTCCTC | 0 | 76 |
| | | | junction | | | |
| 155071 | 52 | 1 | Intron 3/Exon 4 | CAGGAACAACATGGTCGACT | 0 | 77 |
| | | | junction | | | |
| 155072 | 52 | 150 | Exon 4/Intron 4 | ACCGTGGTCCTTCACCAGAG | 0 | 78 |
| | | | junction | | | |
| 155073 | 53 | 1 | Intron 4/Exon 5 | CGAGGAAGGAGTTTACCCCT | 22 | 79 |
| | | | junction | | | |
| 155074 | 53 | 47 | Exon 5/Intron 5 | ACCTGGGAATCCGTCAGCTG | 9 | 80 |
| | | | junction | | | |
| 155075 | 54 | 1 | Intron 5/Exon 6 | GCTCAGTACTTCATAGATCT | 0 | 81 |
| | | | junction | | | |
| 155076 | 54 | 66 | Exon 6/Intron 6 | ACCTCTGCAATGATGTCACC | 0 | 82 |
| | | | junction | | | |
| 155077 | 55 | 1 | Intron 6/Exon 7 | CAGGATCCTCTGCTGTTCCT | 0 | 83 |
| | | | junction | | | |
| 155078 | 55 | 54 | Exon 7/Intron 7 | ACCTCCTCTGGCCGGCTCAG | 0 | 84 |
| | | | junction | | | |
| 155079 | 56 | 1 | Intron 7/Exon 8 | CACAGCTTCAGCCTCGACCT | 18 | 85 |
| | | | junction | | | |
| 155080 | 56 | 106 | Exon 8/Intron 8 | ACCCTTCTTCCGTGCCTGGG | 13 | 86 |
| | | | junction | | | |
| 155081 | 57 | 1 | Intron 8/Exon 9 | CACCATACACCACAGTTCCT | 2 | 87 |
| | | | junction | | | |
| 155082 | 57 | 142 | Exon 9/Intron 9 | ACCAGGACAGCAACGAGTTG | 8 | 88 |
| | | | junction | | | |
| 155083 | 58 | 1 | Intron 9/Exon 10 | GTGACCTGGAGGTCTCCACT | 13 | 89 |
| | | | junction | | | |
| 155084 | 58 | 127 | Exon 10/Intron 10 | ACCACAGCTTTATCCCAAAT | 64 | 90 |
| | | | junction | | | |
| 155085 | 59 | 1 | Intron 10/Exon 11 | GTCCATCTTCCCAGTGACCT | 31 | 91 |
| | | | junction | | | |
| 155086 | 59 | 156 | Exon 11/Intron 11 | ACACTGTTGTGCGTCTTGGC | 6 | 92 |
| | | | junction | | | |
| 155087 | 60 | 1 | Intron 11/Exon 12 | GATGTTGTACTCAAGAGCCT | 19 | 93 |
| | | | junction | | | |
| 155088 | 60 | 165 | Exon 12/Intron 12 | ACCCTGCTCCTTGCCTTGAT | 0 | 94 |
| | | | junction | | | |
| 155089 | 61 | 1 | Intron 12/Exon 13 | CCCCCTCAGCTCAGCCAGCT | 20 | 95 |
| | | | junction | | | |
| 155090 | 61 | 151 | Exon 13/Intron 13 | ACCAGACAAGCTGAAACCAG | 18 | 96 |
| | | | junction | | | |
| 155091 | 62 | 1 | Intron 13/Exon 14 | TGTCGTCAATCTGAGCACCT | 46 | 97 |
| | | | junction | | | |
| 183304 | 55 | 54 | Exon 7/Intron 7 | ACCTCCTCTGGCCGGCTCAG | 52 | 84 |
| | | | junction | 2′-MOE gapmer | | |
| 183305 | 59 | 1 | Intron 10/Exon 11 | GTCCATCTTCCCAGTGACCT | 50 | 91 |
| | | | junction | 2′-MOE gapmer | | |

ISIS 183304 and 183305 (SEQ ID NO: 84 and 91) are lead chimeric oligonucleotides (“gapmers”) 20 nucleotides in length targeted to rat collapsin response mediator protein 2, composed of a central “gap” region consisting of ten 2′-deoxynucleotides, which is flanked on both sides (5′ and 3′ directions) by five-nucleotide “wings”. The wings are composed of 2′-methoxyethyl (2′-MOE) nucleotides. The internucleoside (backbone) linkages are phosphorothioate (P═S) throughout the oligonucleotide.

As shown in Table 5, SEQ ID NO: 90, 91 and 97 demonstrated at least 30% reduction of rat CRMP-2 mRNA levels in this assay and are therefore preferred.

ISIS 155084 (SEQ ID NO: 90), targeted to the exon 10-intron 10 junction of rat CRMP-2, was most active for reducing CRMP-2 mRNA levels in this assay. A dose-response experiment using RT-PCR to measure reduction of CRMP-2 RNA levels in PC-12 cells after treatment with ISIS 155084 showed that reduction of the target RNA was dose-dependent with an IC50 of less than 100 nM. Cells were harvested at 48 hours after treatment for measurement of CRMP-2 protein levels by western blot analysis. A dose-dependent reduction of CRMP-2 protein was demonstrated in cells treated with ISIS 155084.

A dose response experiment was also done with ISIS 155084 in C6 rat glioblastoma cells. Cells were electroporated at 200V for 6 msec, one pulse, and RNA was harvested for RT-PCR at 24 hours after treatment. Again reduction of the target RNA was shown to be dose-dependent, with an IC50 of 1 μM. It should be noted that higher oligonucleotide doses are typically required to see activity (target RNA reduction) in electroporation experiments.

### Example 20

### Reduction of Rat Collapsin Response Mediator Protein 2 (CRMP-2) mRNA Levels by Treatment with Uniformly 2′-MOE Modified Phosphorothioate Antisense Oligonucleotides Targeted to CRMP-2 mRNA Splice Sites—Northern Blot Analysis

The compounds shown in Table 5 are analyzed for their effect on CRMP-2 mRNA levels in PC-12 cells (American Type Culture Collection, Manassas Va.) by Northern blot analysis as described in Examples 13. Data are shown in Table 6 as percent of untreated control and are averages from multiple experiments. If present, “N.D.” indicates “no data”.

TABLE 6
Inhibition of rat collapsin response mediator protein 2 mRNA levels
by uniformly modified 2′-MOE phosphorothioate oligonucleotides -
Northern blot analysis

| | TARGET | | | | % | |
|---|---|---|---|---|---|---|
| | SEQ ID | TARGET | | | decrease | SEQ |
| ISIS # | NO | SITE | REGION | SEQUENCE | in RNA | ID NO |
| 155057 | 48 | 1 | 5′ UTR | AAGAGACAGATGCAATCCTC | 0 | 63 |
| 155058 | 48 | 33 | 5′ UTR | CTGGTCTTGCTATTAGGAGA | 0 | 64 |
| 155059 | 48 | 42 | 5′ UTR | ATCCCTTAGCTGGTCTTGCT | 0 | 65 |
| 155060 | 48 | 63 | 5′ UTR | TATTTGTAGGAAAAAGGTAC | 0 | 66 |
| 155061 | 48 | 89 | 5′ UTR | CTTGGTTTAAAATATATATA | 12 | 67 |
| 155062 | 48 | 117 | 5′ UTR | TTAAAGCAAAGAGAGCCGGA | 4 | 68 |
| 155063 | 48 | 141 | 5′ UTR | GGAAGTAATTTCAAGAGGAC | 0 | 69 |
| 155064 | 48 | 170 | Start codon | CTGATAAGACATCTCTCCGG | 0 | 70 |
| 155065 | 48 | 2888 | PolyA signal | TTGGTGACTTAATCAGGACC | 0 | 71 |
| 155066 | 49 | 199 | Exon 1/Intron 1 | ACCGTGATGCGTGGAATATT | 6 | 72 |
| | | | junction | | | |
| 155067 | 50 | 1 | Intron 1/ Exon 2 | GATCAGAAGACGATCGCTCT | 4 | 73 |
| | | | junction | | | |
| 155068 | 50 | 74 | Exon 2/Intron 2 | ACTTGATCAACCCATCTTCC | 0 | 74 |
| | | | junction | | | |
| 155069 | 51 | 1 | Intron 2/Exon 3 | AGGTTTTCTCCTATTTGCCT | 0 | 75 |
| | | | junction | | | |
| 155070 | 51 | 170 | Exon 3/Intron 3 | ACTGATCATGGTGGTTCCTC | 0 | 76 |
| | | | junction | | | |
| 155071 | 52 | 1 | Intron 3/Exon 4 | CAGGAACAACATGGTCGACT | 0 | 77 |
| | | | junction | | | |
| 155072 | 52 | 150 | Exon 4/Intron 4 | ACCGTGGTCCTTCACCAGAG | 0 | 78 |
| | | | junction | | | |
| 155073 | 53 | 1 | Intron 4/Exon 5 | CGAGGAAGGAGTTTACCCCT | 22 | 79 |
| | | | junction | | | |
| 155074 | 53 | 47 | Exon 5/Intron 5 | ACCTGGGAATCCGTCAGCTG | 9 | 80 |
| | | | junction | | | |
| 155075 | 54 | 1 | Intron 5/Exon 6 | GCTCAGTACTTCATAGATCT | 0 | 81 |
| | | | junction | | | |
| 155076 | 54 | 66 | Exon 6/Intron 6 | ACCTCTGCAATGATGTCACC | 0 | 82 |
| | | | junction | | | |
| 155077 | 55 | 1 | Intron 6/Exon 7 | CAGGATCCTCTGCTGTTCCT | 0 | 83 |
| | | | junction | | | |
| 155078 | 55 | 54 | Exon 7/Intron 7 | ACCTCCTCTGGCCGGCTCAG | 0 | 84 |
| | | | junction | | | |
| 155079 | 56 | 1 | Intron 7/Exon 8 | CACAGCTTCAGCCTCGACCT | 18 | 85 |
| | | | junction | | | |
| 155080 | 56 | 106 | Exon 8/Intron 8 | ACCCTTCTTCCGTGCCTGGG | 13 | 86 |
| | | | junction | | | |
| 155081 | 57 | 1 | Intron 8/Exon 9 | CACCATACACCACAGTTCCT | 2 | 87 |
| | | | junction | | | |
| 155082 | 57 | 142 | Exon 9/Intron 9 | ACCAGGACAGCAACGAGTTG | 8 | 88 |
| | | | junction | | | |
| 155083 | 58 | 1 | Intron 9/Exon 10 | GTGACCTGGAGGTCTCCACT | 13 | 89 |
| | | | junction | | | |
| 155084 | 58 | 127 | Exon 10/Intron | ACCACAGCTTTATCCCAAAT | 64 | 90 |
| | | | 10 junction | | | |
| 155085 | 59 | 1 | Intron 10/Exon | GTCCATCTTCCCAGTGACCT | 31 | 91 |
| | | | 11 junction | | | |
| 155086 | 59 | 156 | Exon 11/Intron | ACACTGTTGTGCGTCTTGGC | 6 | 92 |
| | | | 11 junction | | | |
| 155087 | 60 | 1 | Intron 11/Exon | GATGTTGTACTCAAGAGCCT | 19 | 93 |
| | | | 12 junction | | | |
| 155088 | 60 | 165 | Exon 12/Intron | ACCCTGCTCCTTGCCTTGAT | 0 | 94 |
| | | | 12 junction | | | |
| 155089 | 61 | 1 | Intron 12/Exon | CCCCCTCAGCTCAGCCAGCT | 20 | 95 |
| | | | 13 junction | | | |
| 155090 | 61 | 151 | Exon 13/Intron | ACCAGACAAGCTGAAACCAG | 18 | 96 |
| | | | 13 junction | | | |
| 155091 | 62 | 1 | Intron 13/Exon | TGTCGTCAATCTGAGCACCT | 46 | 97 |
| | | | 14 junction | | | |
| 183304 | 55 | 54 | Exon 7/Intron 7 | ACCTCCTCTGGCCGGCTCAG | 52 | 84 |
| | | | junction | 2′-MOE gapmer | | |
| 183305 | 59 | 1 | Intron 10/Exon | GTCCATCTTCCCAGTGACCT | 50 | 91 |
| | | | 11 junction | 2′-MOE gapmer | | |

As shown in Table 6, SEQ ID NO: 90, 91 and 97 demonstrate at least 30% reduction of rat CRMP-2 mRNA levels in this assay and are therefore preferred. Accumulation of CRMP-2 pre-mRNA is not observed.

### Example 21

### RNase H Assay

In order to determine which antisense compounds are capable of eliciting RNAse H cleavage of their complementary target RNA, an RNAse H assay may be used. One such assay, using cloned and expressed human RNAse H, is described by Wu et al., (1999) J. Biol. Chem. 274, 28270-28278. Similar assays using E. coli RNAse H are well known in the art. For example, Lima et al., 1997, Biochemistry 36, 390-398.

### Example 22

### Reduction of Mouse PTEN mRNA Levels by Treatment with Uniformly 2′-MOE Modified Phosphorothioate Antisense Oligonucleotides

In accordance with the present invention, a series of oligonucleotides were designed to target sequences upstream (5′) of exon/intron junctions of the mouse PTEN RNA, using published sequences. The oligonucleotides, shown in Table 7, have target sites 30 nucleotides upstream of exon/intron junctions. “Target site” indicates the first (5′-most) nucleotide number on the particular target sequence to which the oligonucleotide binds. The mouse PTEN target sequence (provided herein as SEQ ID NO: 98) is a concatenation of mouse PTEN genomic sequence contigs from Genbank accession number AC060781.2. All compounds in Table 7, except as indicated, are uniformly modified, i.e., composed of 2′-methoxyethyl (2′-MOE) nucleotides at each position. The internucleoside (backbone) linkages are phosphorothioate (P═S) throughout the oligonucleotide. All cytidine residues are 5-methylcytidines. The compounds were analyzed for their effect on mouse PTEN levels in b.END cells. LIPOFECTIN/OptiMEM mixture, at a ratio of 2.5 μl LIPOFECTIN to 1 ml OptiMEM, was prepared by mixing and incubating at room temperature for 30 min. 1200 μl of LIPOFECTIN/OptiMEM mixture was aliquotted into 12 wells of a deep well block and oligonucleotide was added to give a concentration of 200 nM. After thorough mixing, 600 μl of the 200 nM oligonucleotide mixture was transferred and diluted into 600 μl of OptiMEM to give an oligonucleotide concentration of 100 nM. The diluted sample was thoroughly mixed by pipetting. The cells were washed with 100 μl of OptiMEM and 130 μl of oligonucleotide mixture was added to each well of a 96 well plate. Cells were incubated for 4 hours at 37° C., 5% CO₂. Oligonucleotide mixture was decanted and replaced with growth medium (GM) with 10% fetal bovine serum. Cells were incubated at 37° C., 5% CO₂ overnight. Plates were washed 1× with PBS and RNA was isolated by the Qiagen RNEASY protocol. Quantitative RT-PCR was carried out as described in other examples herein. Data are shown as percent of untreated control and are averages from multiple experiments. If present, “N.D.” indicates “no data”.

TABLE 7
Reduction of mouse PTEN mRNA levels in b.END cells by 100 nM or 200 nM
uniformly modified 2′-MOE phosphorothioate oligonucleotides

| | TARGET | | | | % decrease | % decrease | |
|---|---|---|---|---|---|---|---|
| | SEQ ID | TARGET | | | in RNA | in RNA | SEQ |
| ISIS # | NO | SITE | REGION | SEQUENCE | (100 nM) | (200 nM) | ID NO |
| 339270 | 98 | 7717 | Exon 1 | AGGGGAGAGAGCAACTCTCC | 3 | 0 | 100 |
| 339271 | 98 | 10534 | Exon 2 | ATCAATATTGTTCCTGTATA | 18 | 0 | 101 |
| 339272 | 98 | 23592 | Exon 3 | CTTGTAATGGTTTTTATGCT | 14 | 0 | 102 |
| 339273 | 98 | 29113 | Exon 4 | AATTTGGCGGTGTCATAATG | 15 | 20 | 103 |
| 339274 | 98 | 31098 | Exon 5 | TGGTCCTTACTTCCCCATAA | 17 | 15 | 104 |
| 339275 | 98 | 34688 | Exon 6 | CCACTGAACATTGGAATAGT | 7 | 0 | 105 |
| 339276 | 98 | 38433 | Exon 7 | TCTTGTTCTGTTTGTGGAAG | 8 | 0 | 106 |
| 339277 | 98 | 40910 | Exon 8 | GAGAGAAGTATCGGTTGGCC | 7 | 0 | 107 |
| 339278 | 98 | 43537 | Exon 9 | AGGACAGCAGCCAATCTCTC | 2 | 0 | 108 |
| 116847 | 99 | 2097 | human PTEN | CTGCTAGCCTCTGGATTTGA | 87 | 87 | 109 |
| | | | Exon 10 | 2′ MOE at positions 1-5 | | | |
| | | | MOE gapmer | and 16-20, 2′deoxy at | | | |
| | | | | positions 6-15 | | | |
| 129700 | Control | | Scrambled | TAGTGCGGACCTACCCACGA | 21 | 42 | 110 |
| | | | Control | 2′ MOE at positions 1-5 | | | |
| | | | MOE gapmer | and 16-20, 2′deoxy at | | | |
| | | | | positions 6-15 | | | |
| 129695 | Control | | Scrambled | TTCTACCTCGCGCGATTTAC | 19 | 12 | 111 |
| | | | Control | 2′ MOE at positions 1-5 | | | |
| | | | MOE gapmer | and 16-20, 2′deoxy at | | | |
| | | | | positions 6-15 | | | |

ISIS 116847, 129700 and 129695 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE) nucleotides. The internucleoside (backbone) linkages are phosphorothioate (P═S) throughout the oligonucleotide. ISIS 116847 is targeted to human PTEN (provided herein as SEQ ID NO: 99) and ISIS 129700 and 129695 are universal scrambled control oligonucleotides.

As shown in Table 7, a number of uniformly modified oligonucleotides caused reduction of PTEN target RNA levels. At a concentration of 100 nM, ISIS 339271, 339273 and 339274 (SEQ ID NO: 102, 103 and 104) demonstrated at least 15% reduction of mouse PTEN RNA levels in this assay and are therefore preferred.

### Example 23

### Reduction of Mouse CD40 mRNA Levels by Treatment with Uniformly Modified PNA Antisense Oligonucleotides

In accordance with the present invention, an oligonucleotide was designed to target the sequence upstream (5′) of an exon/intron junction of the mouse CD40 RNA using published sequences from Genbank accession number M83312.1 (provided herein as SEQ ID NO: 112). The oligonucleotide, shown in Table 8 and designated ISIS 208529 (SEQ ID NO: 114), has a target site 15 nucleotides upstream of the exon 6/intron 6 junction of mouse CD40. “Target site” indicates the first (5′-most) nucleotide number on the particular target sequence to which the oligonucleotide binds. ISIS 208529 is uniformly modified with PNA replacing each sugar and phosphate linker and additionally contains a 3′ Lysine side chain. The control oligonucleotide (ISIS 256664) is targeted to the 5′UTR of cytokine-inducible SH2-containing protein (provided herein as SEQ ID NO: 113). ISIS 256664 (SEQ ID NO: 115) is composed of 2′-deoxyribose at each sugar residue, a phosphate backbone, a 5′Fluoroscein and 3′TAMRA. The compounds were analyzed for their effect on mouse CD40 levels in primary macrophages.

Primary thioglycollate-elicited macrophages were isolated by peritoneal lavage from 6-8 week old female C57Bl/6 mice that had been injected with 1 mL 3% thioglycollate broth 4 days previously. PNA oligonucleotides were delivered at a concentration of 1.1 μM, 3.3 μM or 10 μM to unpurified peritoneal cells by a single 6 ms pulse, 90V, on a BTX square wave electroporator in 1 mm cuvettes. After electroporation, the cells were plated for 1 hour in serum-free RPMI 1640 (supplemented with 10 mM HEPES) at 37° C., 5% CO₂ to allow the macrophages to attach. Non-adherent cells were then washed away and the media was replaced with complete RPMI 1640 (10% FBS, 10 mM HEPES). Following overnight incubation at 37° C., cells were washed 1× with PBS and RNA was isolated by the Qiagen RNEASY protocol. Quantitative RT-PCR was carried out as described in other examples herein. Data are shown as percent of untreated control and are averages from multiple experiments. If present, “N.D.” indicates “no data”.

TABLE 8
Reduction of mouse CD40 mRNA levels in primary
macrophages by uniformly modified PNA oligonucleotides

| | TARGET | | | | % decrease | % decrease | % decrease | |
|---|---|---|---|---|---|---|---|---|
| | SEQ ID | TARGET | | | in RNA | in RNA | in RNA | SEQ |
| ISIS # | NO | SITE | REGION | SEQUENCE | (1.1 μM) | (3.3 μM) | (10 μm) | ID NO |
| 208529 | 112 | 553 | Exon 6 | CACAGATGACATTAG | 29 | 44 | 63 | 114 |
| 256664 | 113 | 115 | 5′UTR | TTCCATCCCGCCGAACTCC | 0 | 0 | 0 | 115 |

As shown in Table 8, treatment with ISIS 208529 resulted in a dose-dependent decrease in levels of CD40 mRNA in primary macrophages. Thus, antisense oligonucleotides modified with PNA, which are not able to recruit RNAse H for cleavage of target RNA, are able to reduce target mRNA levels in a sequence-specific manner.

What is claimed is:

1-26. (canceled)

27. A method of decreasing the amount of a preselected human cellular mRNA or corresponding protein in a cell comprising:
contacting the cell expressing the preselected cellular mRNA with an oligomeric compound comprising a modified oligonucleotide consisting of 12 to 30 linked nucleosides having a nucleobase sequence that is specifically hybridizable to a target region of the preselected mRNA selected from the group consisting of an intron/exon junction, an exon/intron junction and a region 1 to 50 nucleobases 5′ of an exon/intron junction,
wherein each nucleoside of the modified oligonucleotide comprises a modified sugar moiety comprising a modification at the 2′-position,
wherein said oligomeric compound is not a substrate for RNAse H when bound to said preselected mRNA, and
wherein the amount of the preselected mRNA or corresponding protein is reduced.

28. The method of claim 27, wherein the target region is selected from the group consisting of a region 1 to 15 nucleobases 5′ of an exon/intron junction, 20 to 24 nucleobases 5′ of an exon/intron junction, and 30 to 50 nucleobases 5′ of an exon/intron junction.

29. The method of claim 27, wherein said 2′ sugar modification is a substituted or unsubstituted 2′-O-alkyl, substituted or unsubstituted 2′-O-alkyl-O-alkyl, 2′-acetamido, 2′-guanidinium, 2′-carbamate, 2′-fluoro or 2′-aminooxy modification.

30. The method of claim 29, wherein said substituted or unsubstituted 2′-O-alkyl modification is a 2′-O-methyl modification.

31. The method of claim 29, wherein said substituted or unsubstituted 2′-O-alkyl-O-alkyl modification is a 2′-O-methoxyethyl, 2′-dimethylaminooxyethoxy, or 2′-dimethylaminoethoxyethoxy modification.

32. The method of claim 27, wherein the modified sugar moiety is 2′-O-methoxyethyl.

33. The method of claim 27, wherein said modified oligonucleotide comprises at least one modified backbone linkage.

34. The method of claim 33, wherein said modified backbone linkage is a phosphorothioate, 3′-methylene phosphonate, methylene (methylimino), morpholino, locked nucleic acid, or peptide nucleic acid linkage.

35. The method of claim 33, wherein each modified internucleoside linkage is phosphorothioate.

36. The method of claim 33, wherein the modified backbone linkage is peptide nucleic acid.

37. The method of claim 35, wherein said peptide nucleic acid is bound to a cationic tail.

38. The method of claim 36, wherein said cationic tail comprises one to four lysine or arginine residues.

39. The method of claim 33, wherein said modified oligonucleotide comprises a modified backbone linkage at every linkage.

40. The method of claim 33, wherein said modified backbone linkages alternate with phosphodiester and phosphorothioate backbone linkages.

41. The method of claim 27, wherein each nucleoside of the antisense oligonucleotide comprises a 2′-O-methoxyethyl sugar moiety and each internucleoside linkage is a phosphorothioate linkage.

42. The method of claim 27, wherein said modified oligonucleotide comprises at least one modified nucleobase.

43. The method of claim 41, wherein said modified nucleobase is a 5′methylcytosine or a C-5 propyne.

44. The method of claim 41, wherein each cytosine in said modified oligonucleotide is a 5-methylcytosine.

45. The method of claim 40, wherein each cytosine in said modified oligonucleotide is a 5-methylcytosine.


SEQUENCE LISTING (115 sequences)

SEQ ID NO: 1 - DNA, 76698 bp, H. sapiens
  antisense oligonucleotide
cgcggaattccagacctcaggtgatccacccacctcggcctcccaaggtgctgggattacaggcgtgagccaccatgcctggccgattgttccaatgtatatgcaccccagtaatttatgagagagcccaggtcttaatttttaattgttttccaagatggctgtactaggctttcctgcaaatgacaccatagcatatattgtggttgccaccccagcaaccaggccctcaccctccatcatgggctgcccattatggcatgagggggattgacactgggccaggtatcttttgcccctctaggattccccttcatcattctctccatgccgtctgccccaggaaggcgatctccaacctcagagacctgcttgctgtttcccaaacttatgctaatcacacctctatgcctttgcccatactgttcccacctcttgccctgcactccttcccttctcagtctggaacattctgaagttgtcctcacaggattaacaagaattttggacaaaaatatattaatagttataattaagcattacttaggctgcactttgacccactttcttgtaactgaaaattacagggcactagatactgaccatttgcatccccattgttcctacagataggtttttttttttttttttgacaaggtctcactctgtcacccaggctggagtgcagtggtacaatcatggctcactgcagtcttgacctcccacactcaagcaatcctcccgcctcaacttcctgagtagcccagtctacaggtgtaggctaccacacctcgctaatttttaaatttttttgtagagacaggggtctccctatgttgcccagaatggtcttgaactcttgggctaagaggtcctcccacctcagcctcccaaagtgctaggattacaagtgtgagccgccaccacacctggcctatagatcagctttctgatgctagaataataagccttttatttaagataggtagaatctctgacattagaatcataaggtttttgtttaagaatttcttaagatgttttttagatcctgaattccagcaagacagctgacctcaaatagtctgaagacccactgacccctacagaggaatggaatcagcatgagaatacagtttcttcatctccctgttccatgactttgccctgtgccctttgagcaatcaaggatctccacactttggctgattcccaaacccctgaaaaccctagccccaaactctgtggagacggatttgaggtttcctcccatctcctggttcagcatccctagaaataaacctctttcactgctgcaatgtggtgaattgacttgccacgtgcaccggataaaggacctattatggttacaattccactcatcctttaagatagcttatatgttgtctctggtcactgcctccctcctcttggtgcccctcgcacagttatccatgagagcacatttgcgtcacctgctggggcaactgtttgtttacatggctctgtctctcccagcacccagcccaggccagccccacacttcaaagtccctgcagggcaggatggcatggaaaggtcacaggtttgggagtcagactgaatatgactccaccctctgtcctcagcctcatctgctcccccagttttctgtgctctaaccacactggcctgcactcctgtctcacttcatggcccttatacatgctgttccaactgcttagaatgctcttcctctggctctttttcatcctttcgtgcccagcttaactatcacctcctgagacaggccttccttgactactgaatctaaaggcacaccctcttcccattctgtcattctccagcaattcccttcattgatttgccacaaccctaattatcatattattcatttacttgtttgctgcttgtctcccctgctagagcttaaagtccttgagtacatacagggactttgccttgtttactgctataggcccagctctaacacagggcctggcatatattaagtattaaaaaaatttaattttagctttttttttttttttgtgaacggagtttcgctcttgttgcccaggctggagtgcaatggcacgatctcgactcaccgcaacctctgcctcccgggttcaagcgattctcctgcctcagcctccctagtagctgggattacaggcatgtgcctccatatctggataattttgtacttttagcagagatggggtttctccatgttggtcaggctagtctcgaactcccgaactcaggtgatccacccgcctcggcctcccaaagtcctgggattacaggcatgagccactgcaagcggccaattttagcttttttcagacaagctggagtgcagtggcatgatcatagctgactgcagcctctaattcctgggctcagctgatcctcctgcctcagcctcccaggaagctagaactacaggaatgtgccaccacccctggctaattttaaaaatttttgatagaaatggagtctcacgatgtagtccaggctggtctcaaactcctggtctcaagtggttctctcactttggcctcctgaattgctgggattacaggtgtgagccaccagtccaccaagaaatttttattaactgaatgaggaatgaacaaacaaaatagatccaaatccttgctccactacttaccaccagatttgtgtcttaggacaaattacttaccctctcctcatgtgaagatgaggcctctcatgggttgtgtattggaaactgtaaaaatgcctgatacgtgaagacattccataaatggccgttattttttctttccttcatctgaaaaatgtaccctttttgccaagcataaagaccttactgtacatctttactttttcttttcttttttgttttttgagatggagtctcgctctgtagcccaggctggagtacagtggtgtgatcttggctcactgcaagccccgcctcctgggttcacgccattctcctgcctcagcctccggagtagctgggactacaggcatccgccaccacgcccagctaattttttgtattttgtttagtagagacggggtttcactgtgttagccaggatggtctcgatctcctgacctcatgatccacccgcctcggcctcccaaagtgctgggattacaggcgtgagccaccatgcctggccaacggtacatcttttttttttttttttttttttgagacagggtctccctctgtcgcccaggctggagtgcagtggcacaatcttggctcactgcaacctccaactccccggttcaagcaattcttgtgcctcagcctacagagtagctgggactacaagcatgcgccaccatgcccagctaatttttgtatttttagtagagatgggattttgtcatgttggccaggctggtcttaaactcctgacctcagatgatctgcctgcctcagcctcccaaagtgttgggattacaagcgtgagccactgcgcccggcctattttcctcctctgatctgacatcatgggcatgtctattcttccttcaaaccatttcagactcattccttcctcctattactcttctgagacctttcctaataactttagcacacttgacctctcctaccaccaaaccagaggtatctaaagtaggggatatgcaacccagcatgtaacacacatgttttagcacacacgatgcccaaaaaatggaaacagcccaaatgtccaccaacagatgaatggataaacaaaatgtggcataaacttacaatgggatattattcagccatgaaaatgaataaagtactgacacatgctaccatgtggatgaaccttgaaaacattatgccaggtgaaagaagtcagtcacaaaaggccacatattgtgtgagtccatttttatgtaatatccagaatagaaaaatccatagtgacagaatgcatattggtgattgccagacgttcaggggatggggaagaaactgcttgatgggtaaggggttttactttggagtaatggaaatgttttggaactaggggtggtggctgtaaaagactgaatgtactaaatgccactaaatgttcagtttaaaatggttcatttcacctcaataaattttttaaaaaatgaagtagccattcttccaggtgagctgaaaagtttgaatgaggcacaggctccttaaatttcttttttttttttttttttttttttgagacggagtctcgctctgtcgcccaggctggagtgcagtggcgcgatctcggctcactgcaagctccgcctcccgggttcacgccattctcctgcctcagcctcccgagtagctgggactacaggcgcccgccactacgcccggctaattttttgtatttttagtagagacggggtttcaccgtgttagccgggatggtctcgatctcctgacctcgtgatccgcccgcctcggcctcccaaagtgctgggattacaggcgtgagccaccttaaatttctaagatgtaaagtgctgggcaaatatcagctggggatgctgaaggaaggaataatcagaaggtcagcaagtgtggcttcgaaactctgcctcaagtaataatgataatgataattagagatagttataatattgacttctttggtttccttgtaaaccagtgttattttagaaaaagagggagatagctctagtaattacagctaacacttctacaatgcttaatatgaggaaggcactgttccaagtactttacgtctaaaacttactaaatccttacaactctaagaggtagtatcatcacatttccattatagatgagggaatggaagaattgagaagtttaaatgagttctccaagtcacagataaggaaatggcagagtccaaatttgaacccaggcaagtcagactctaggcactgaagtctcaaccaccaggctctgcactaagtgctctccaggttttatctcatttaatcctgcaaggaaagtgttattattcccattttattttatttattatttatttatttatttattgagacggagtttcacccttgttgcccaagccaaagtgcaatggcacaatctccgctcgctgcaacttctgcctcccaggttcaagcagttctcctgcctcagcctcccgagtagctgagattacaggccaccatgcccggctaattttgtatttttagtagacatggggtttctccatgttggtcaggctggtctcgaactcccaacctcaggtgatctgcctgcctcagcttcccaaagtgctgggattacaggcatgagccaccgtgcctggcctattattcccattttaaaaatccccctcatgctatccacattccacaccttctagtctttcttttttttttttttttttttgagacggagtttcgctctgtcgcccaggcagacggagtgcagtggcgccatcttggctcactgtaagctctgcctcctgggttcacgccattctcctgcctcagccttccgagtagccgggactacaggcacccgccaccacacccggctaattttttgtatttttagtagagatgggatttcaccgtgttagccaggatggtctcgatctcctgacctcgtgatccgcctgccttggcctcccaaagtgctgggattacaggcgtgagccaccgcgcccggcttttttaaaaatttttttattttttttatttttagtagagaccgggtttcaccgtgttagccaggagggtctctatttcttgaccttgtgatctgcctgcctcggcctcccaaagggctgggattacaagcgtgagcgaccgcgcctggccagtctttctcctacatttatttttacgttggtccacatactcctgtcattctcactttgcttcacttttcctttcttcttcttttttaagagacgggggcttgctatgttgtccaggctggagtgcagtgaggcaatcatagcttatgccatccccaactccaagtgatcctccagcctcagcctcctccctagctggattacaggagcatgtcaccatgcacactaattttcttttcttttttttttttggtagagatggggtctcatgttgctcaggctggtcttcaacatctgggctgaagtgaccccccttccttggcctctcaaagtgctgggattagaggctttggccaccacatccaacctgaattttattatttatattttcttttaatctcccattactagatggcagggattttgattactgttaattttccaatatccaaaataatgtgtggtacctaataggctctcaatatcgaaaagtaatagtgcacatggcattctgtagtattaggtaggtatcttgtgttcctgtgtttgcgtaaataagatcatacattatgttctgcttttttaacttaatggctttttttttcctttttttgcgacagagtctggctctgtcacctaggctggagtgcagtggcgctatctcggctcactgcaacctctgcctactgggttcaagtgattctcctgcctcagcctcctgagtagctgggattacagacgcgcaccaccacacctggccaatttttttttttttttttttaggcggagtctcactctgttgtccaggctggagtgcagtggcgcgatctcagctcactgcaagctccgcctcccgggttcatgccattctcctgcctcagcctcctgagtagctgggactacaggggcccgccaccacacccggctaatcttttgtatttttagtagagacggggttttactgtgttagccaggatggtctcgatctcctgacttcgtgatctgcccgcctcggcctcccaaagtgctgggattacatgtgtgagccaccgcacccggcctatttgttttgtattttttagcagagacaggtttcaccatgttggccaggctggtctcaaactcatgacctcaagtgatctgcccgcctcggcctcccaaagtgctgggattacaggcatgagccaccacgcccagccatgtcttttttttttttttttttgagacaagagtttcgctcttgttgcccaggctggagtgcaatgacgcgatttcggctcaccgcaatctccgcctcctgggtacaagcaattctcctgccttagcctcccgagtagatgggatgacaggcatgcaccaccatgcccagctaatttggtatttttatttttttatatttatttattttttcgagacggagtctcgctctgtcgcccaggctggagtgtaatggtgcgatctgggctcactgcaacctctgcctcccgggttcaagcgattctcctgtctcagcctcctgagtagctgggattacaggcgcccgccaccacgcccggctaatttttgtatttttagtagagacggggtttctccatgttggtcaggctggtctcgaactcccgacctcaggtgatccgcctgcctcggccttccaaagtgctgggattacaggagtaatcccaaaaaaagcgccgggccctttttttgttgttttttaaattcagtaactatctagttcattcttggatggatgacaacccagattggatgtgtagcagcgttctcttaaccagtttcctattaatcttcatttcatccccagtgtttctccagaatgcaaataatatggcattaaatatcttcacacatagctttttgtgtatgtgtatacttatttctctagaattagtgtctagaagtgaaactgccgggaggaaggatatatacttttaacatgtccaagttccactgtgatagcgctgcgagggcacacaacaggtttcaatataccttggaccaaaccggatattatcagtttttttaacttgttgctaatgtgatgggggaaaaatgaactcggaatttacacacaaggaaaagaccgtttaaggttcagggactgtccacatagctgtcaagtggcggagccgtgatttggtattaaagtgcccggagaggacgcgtcaaagttggacactgtgccctgtgtcctgaggcacgtctggtgatcgctgggccttgcaatgctgggcaggcaggccttcctctccccttctaggcctctggccactcctggctggccgaaagccggttcttctcgattaccgagtgcctctcctgaaagcaagtcagcgtcgcctaacctcttcagcttcgaaatggcggccaccagatcgctaggccacgccccgggggcggggcctgagttcaggccagagcgatggatgcccgagccaagttagaagtcgactgccagtagggctcgcgcagaatcggagagccggtggcgtcgcaggtcgggaggacgagcaccgagtcgagggctcgctcgtctgggccgcccgagagtcttaatcgcgggcgcttgggccgccatcttagatggcgggagtaagaggaaaacgattgtgaggcgggaacggctttctgctgccttttttgggccccgaaaagggtcagctggccgggctttggggcgcgtgccctgaggcgcggagcgcgtttgctacgatgcgggggctgctcggggctccgtcccctgggctggggacgcgccgaatgtgaccgcctcccgctccctcacccgccgcggggaggaggagcgggcgagaagctgccgccgaacgacaggacgttggggcggcctggctccctcaggtaggtggcaggaccgggtcgtggatgccgggggagccgggcggcggggctgagggatcggcttccagggcgaccgggcctgggtggcgctgatggagcggccccgcggctgccgggcagagggcttgggccaggccgttgtcaccctggggtagcgttgggcgggggccccggagtccggtgtcatggccggcgagccgagttcccacatcccactcaaatttccttgtgtttggcggaaacgtgccaacgccacccttatgccatgcgcattcctcatatttggcagtgggaaaatccgcccagagctgccccatatctgttgtcacttggatgggccaattccttttctcttgggccgccgaatgtgggacccgggcttgcaccctttctcagggtacttcagtcaagtgacacccttttagagacgacgtgaggaatcgggtaagagaggaggaaactggccagtgccctaccacaaaggcacaggggcctcttcttgggtatcaggactagccttgggtatcaggactctgggttattaatgaaaggtttgggatacttatagaggattggcctcaggacgctttggaatgaagagccagggctgtcttttgtgtgacgcgagagccgccgggacgcttcagctctgcagctgctgaggctctgcgagcgagtcgatgcccaagagagaggggtttggacgtcgtgagaggcgaggcggccgtgttcattcattgttctcgttctagggctctgggtgtgcccctggtattcattctgtggtgggaagaaggaatggaacttagtgtatccttgagatgtgaacgggttctagggggtcacttaatctaagtggaaaatgaattcaaggcacgttcattgagcgtttctgcttgcctggtcctctgtgggctgagtggagagactctgccctccctgcgctcctaaggcgtgaaaacaatgcagtgtgataagaattggcttatcaagtgttatggggatttagaacagttagttttgcttggggaggagttgaggaagcttctacactcgaggagacttctgagtcgagttttgaaacacctgtgagtaagtgctcatcgggtgaggaggagctcagggaacagctggtacaaaggcttagagccatgtgggagttgggatgagtttggggagcagcaaattgcctggggtgcaggaaggaaatggtgagagatgagagtaaaataaaagttgctagaattgtgagggggctgtctttgttgtagatagtgaactagttgaatttggattattgtacatgggttgccgagtcttcattcttgctgataattttctccctttgttgatgttgaagctgatagtgattgaacatatttagtttaacttagttaatgacttttaaatttttttttattttttcagaacaatgcaaactttttttttttttttttttttttttttttttttaaaggaacaggatctcactctgtcgcccaggctagagtgcagtggcatgatcatagctcggttgcagcctctaactcctgggcttaagcagttctcctgcctttgcctcctgagtagctgggactacagacaggtgccaccacacatggctaattaaaaaaaaaatagtagagatggagtctggcagtgttgcctaggctggtctcaaactcctgggctcaggcgatcctcctgcttccacctctccctcccaacgtgcttgctgggattacaggggtgagccactggccaggcagaactttttttttttttaaataatagagagggggtcacactatgttggccaggctggtcttgaactcttgggctcaagtgatcctccagcttcagcctcttaaagtgctgaaattacaggtgtgatccactgtgcctggctagcagaacatttttgataagtgttttatatcaaatgttttgacttacacagtggtgaatgaattgaactcatatattcctggggattcttgcaaaaaattctcttaaagttatacttgctcacaaaaatgttaactttataaatgtagaacactctcctactaatttttattttattattctattgttttttatttttttgcgacggagtctcactctgttgcccaggctggcgtgcaatgatgcgatctcggctcactgcaacctctgcctccttggttcaagcagttctcctgcctcaccctcctgagtagctgggtaggcacactccaccacgcccggctgatttttgtatttttagtagagatggggttttgtcgtgttggccaggctggtctcgaactcctgaccgcaagagatctgcccacctcggcctcccacggcctcgctgggattacaggcatgagccactgtgcctggcctaaattttaaatataagtaatgtactccccagtcttacagaaattggacgactatagaaaacaaacatcaaaaaaagtgtagaatgtgagtatttttagtttaataagtgtattttataaactatttatttgtattgacttctcggataacaacctgttataaaatctttatccccataaacataattttcctaaaatagctataatattgtgattaatgtttatgctaaagtgactattatggaattaacagacttcagttgcagtttctaaatcttgctttggttgtgatgattatataccactgaagaacattcaggattattttggcttgtttttacccttatcactcaagggctaagctgtttaaaatgcaacataaacatttgacccagttgaatgctgggatacttggaaaaataaacctgttactgtttctgtactaaaggcttatcttttaaagatatgtggtgtttttttagcgcagtggtgcgatcttggctcactgcgacctctgcctcctgggtttaagcattctcctgcctcagcctcctgagtagctgggactacaggcgcctgccaccacgcctagccaacttttatgtttttagtagagacgggatttcaccatattagccaggctggtcttgaactcctgaccttgtgatctacccgccttggccttgcaaagtgctgggattacaggcgtgagccactgtgcctggctgatatgtggtgttttgtgattataaattgtagtggagttccttagttttgttaaagtcttgtcagtagttgtaaaaacatcagccagttgtggtggctcaggcctgtaagcccagcactttgggaggccgaggctggtgaattgctagagctcaggagtttgagaccagcctgggcaacatggtgaaaacctgtccctacaaaaaatacacacacacaaaaagaaaaaaatcagcagggtatggtgtagtatgcctgtagtcccagctgcttgggaggctgaggtgaaaggctcacctgagcccagggagattgaggctgcagtgagccatgttcatgccactgtactccagtgttggtgatggagtgagaccctgtctcaaaaaaaaaaagtgtgccttcaatagaaggcttgaacgtattttatgggatttggtttagctgaaaaaaacagtgagaagcagattaagctggtaatttctgacaaaaagtatctaaaagatgaagtgaagaatgttaaacatcaagtattatattacagttgctcttagactagtagcttttagtttataacatgtcatttgtttgctctgaagattaagcaagttcatacttcttggaagttaaatttgacttttccagaagcactggattatttacgaaataaaaaatataattgataactttaaactactatttcaggtagtctattactagtaaatgtatgattctacatttaaatttcaggtaaatctttgttagtaacctactgcctaaaaaaatgttacatgagggagtacttttgtttgcatgttaggatcataataggccatacataataatcttgagcttgggaggagcttgttagccaaacagcatgccttaatgttgacttgcagaagacaattttaaatattgcctttgaaaggcagtggataatgtgacagtgagggggtttatgaaaccataaaattgagctttttgacttagtttttgtttttaagttgttcagatcttgggagtcatttcttcaaaacaaatgactatgaggtggaaaattacttaccttgaataaattaattggaaaatcagagaacactgggtttatttaggatgaggttgtttggtatgtgtatgggagggtagaattcctaattgctcatctgactgggttcaaaatgtaatactagatatttgtgttgcaattcagttggtacttttggtatagggctaacttatcttgcgtgtaattttttttttttttttttgagatgaaatctggtgctgttgcccaggctggagtgcagtggtgtgatcttggctcactacaacctccgtctcccaggttcaagggattctcatgcctcagcctcccgagtagctgggattacaggcgccggccaccttgcctggctaatttttgtatttttagtagagacgaggtttcaccatgttggccaggctggtcttgaactcctgacctcaagtgatccacctgcctcggcttcccaaagtgctggcattacaggctcgctcaggcatcttgccttgtaattctcatgatagtaatggctatttttttcttgccttagagttgtaagtaaaaattccttaattacacattaaggtttgatctttaattttacaatgtttgagtcattttgttacttcttttctcccagaatgacttgcgtagctctaaatgattttagttaatttcacatctgtttgcctttcttctaaaatgacccctagaatctcagcttaactaaggaaaatgtcaagtgggtgttgtttctttgttagtggttttggcctagactatctaaagtttggcaaattactcacaaagtatgttaattggcatcacattccaatcagtgtacatagcattttttgaggaacacttgacacacggttttatttttagaccagattctaaggggttttactgggtggggcttaacaatcctaaagctagtttacggttttaaaatctttatgatttagaggttgtttacattttttgttaataaatgggaagcagcaggcagtggcagtcaattttgtttgtttctttttttgttttttttgagacggagtttcgttcttgttgcccaggctggagtgcagtggcatgatctttcctcaccacagcctctgcctcctgggttcaagcgattctcctgcctcagcctcctgagtagctgggattacaggcatgcgccaccacacctggctaattttgtatttttagtagagacagggtttcactgtgttggtcatgctggtcttgaactccctaactcaggtgatctgcctgcctcagcctcccaaagtgctgggattacaggcgtgagccaccacgcccagccctcacataacttttatgatattatgttcttataattgttccattattaattataattaatctctcactgtgcctaatttatatgttaaacttgatcatgggtatgtatgtacaggaaaaaacatagtgtatacagtatagtatactgttcttgctttcaggcattcattggtagtcttggaacatattccaagtggatatggaagcactactatgtgatggaatgttactcagtaataaaaagaaggatgtactggtgtatactacaacattggaaacatattaagtaaaagaaaccatgcaggaaagaccacatattgaattattccatttatatgtaatgtccagaataggaaaatccttagtgacagaaagtagatcaggggctgagggatgtagggaatggtcagtgactgtgatagggttttctttttgcttttgacagcggtctgcattcataattgctaatacttggaagcaaccaagatgtccctcagcaggcgaatggaaaaactggtacatccagacaagggactattgttcagtgccaaaaagaagcaagataccaagccatgaaagacatggaggaaacttaaatgcatatcactgagtggaagaagccaatctaaaaaggctgtatacggtatgactcccaactatatgaaactgtggaaaaggcaaaactgctgagacaggaaaaagatcagtggttgacggaagggagggatacataggcagagtacagagaatttttagggcggtgaaactactgtaatatgtcattatacatttgtcaaaacccatagagtaagcctgggcaaaatagcaagaccccatctctaccaaaaatttttaaacctagccaggcacttgtcctccaaaagcccacttggccctcttcaagtatattttactttcttttccttcctgctctgaagctttttataacctttcatgctgctggaaaacttgcctcagtttctttatcttgcctatgcccctcatccaattccttcttctgaggaggcaaaaatgagggtcgtgcagcctgcacggatcacttgccggaaactcgacacccgcacgcaaaataattcggggtgcgctcactannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaagnaaaaggttaggaaacattaacttagcctgcctcttttttttttttttttttttttgagacagagtctcgctctgtcgcccaggctggttggagtgcagtggcatgatctcggctcactgcaagctccgcctcctgggttcatgccattctcctgcctcagcctcctgagtagctgggactacaggtgcccaccaccacgcccggctaattttttgtatttttagtagaggggtttcaccctgttagccaggatggtctccatctcctgacctcgtgatccatctgcctcggcctccctaagtgctgggattacaggcgtgagcccccgcacccaacccttagcctgcctcttaagctgtaagtggtcttgatatggagatagaaaataaaatactatgaatgacaaataatctaaaacttgaattaaataaagtaggtgtatttttattttgtcactttttattaaaagttattgcagtatattctctactgagtaccagcactatattttgagtgcctgcaagacttagaattcattgtaaaattactgttcttggactgaggttacattttagtcttatcagtggattcttcaccaatcgattggaatcagtcaattccaatacagtcttcccccacagttgaatatagaataaaatctattgcaagctgggtgcaggggcacaagtgtggcaggagtgcttgagcctaggagttcaagaccagcctgggcaacatagtgagacctcatctcaattgaaaatatatatctatataaaaaataaaatttattacagttcatcttgctggaaaacaaaatactgtttttgtaattaaaattttttttttaaatttagaaatggggtcttgctgtgttgaccaggctggtcttgaactcttggcctcaagctgtcctcccatctgggcctcccaaagtgctgggattacaggtgtgaacaactgcgcccggctgacaaagtattttttaaagatgtaccactaaatggagatttgattcacatttgatagtttttgacaggtcttttctatttaaaaacattactgtttttgtagcattattctggcttttcccttaatttagtaaatatttgagtgcctttgtattccagatactgagcaagattggcagggttctgcccttatggagcagaaggaaggtagggggactgactaaaacttgaaaactgtctaacataagtaccatgcagaaaatgaaacagtattaattggcagaaggagagcaggctattttggctagtgtggttagggaaagcctctctaaagagatgtctcttgggtggagacaagatgtgaaaaaaccagcttgcctgtttttggggtttcagccttgcaggtgaagagaaacacgaagttcagaagtcttgaggcacaaagtctggcatgttacgaaagaaggcctttagacgccttgtcagggagtttagattttattctgagttttaaaacgggagtgacacaatgagttgcattttaagcctgttcaggctgttacatggattattaggagctgtatcatttcaggctagtgagatgctcagatgagtctgccttctgtctcttccgtcatctatttctctcttatctggtcttaagctcctccatcttttcctttttagttggaaaaaaactcaaagatctagaaaaaagaggagctgtatgtactcctaaaaagggacctcatagtaacctggggatagagttatgtaggagtgagtcagggctcaggttgaggctttagaggcaggaggcagcgagatcttgttctgtcatcccctcttacagaaataaaatatgccgataaaagtttatagtgtaatagtaaaatataaaaacaaaaagtaagtaatgtagaaaataaaaacccttcacagtcctgctgaaatgattactgttaacactttaattctagagttccccatccatttatttatttctagatttccctctttgtagattaatattaaagggttcagacttgttcattttttgttgtcttggatatcttttcccacctctgtatatatggatctactttatttatcacgtggatattaacatggtttatttaattccctattgttaggtatttggtctttaccacagtttttcaagggtatgaatagtgctgcaaggaatatgcttacacatgtttttatacacttgtcttaggcttctgtaggacaaatttctggagtagaatactaggtcattctttaagaacatttcaaacttttaatagatattaccgtattctttcccaaaaagaatgtacaaagactgtatgagaataactccatgttgtgatcttaagttgtctctaaacctctttggttttcttagctgtcatctaagaatactaagtatctaacctccctcttgatttgggcatgtgatgtgatttagcatatagtggatattcagttagaaacttttggttgaaaacaaggtttggattctgtggtctttaattctaggccatttcagctctgactaaaatgatttgagtgttagtgttatatatgggaaggtaagggctatggagtcagtgcagcccagttcagaatcccagtttgccacttacaagctgtgtgtgtgagaattttctcaactgtaaaatggggacataattcctacctagagtaatactgtaagtattaaggtggataatgattggaatgtatgctgtgtatcctgcctcataatagtaagcttttagtaaatggtagctactgttaataataaaacaagtttctgaaggaggaaggcttgaaaagatgggattccttatcaacctcaaagttttctaaaggaggaaaccctaccccccttacttctgcatggtttctgaccatgaactgaactctgaactctgaatgaactgaactctgaactctgaatgaactgaactctgaactctgaatgttatggtagaaaattcatggactttaaatttaaacagataaagaatctggttattttacccactgctggggtgttcttgggcaagtagcatgacttctgtgtccaaaaaagaaagggtttgcagtgactgaacctgtaatcccagtactttgggaggctaaggagagtggattgcctgagctcaggagttcaagaccagcctgggcaacatagtgagagcctttctcaacaaaaaaaactgttcttaaaaattagctgggcatggtgatgcacgtctgtggtcccagctatgtgggaagctgaggtaggagaatcatttgagcctggaaaattgaagctgcagtgagctgtgatcatgtcactgcaccccagcctgggcaacagagcaagaccctgtctcagaaaataaattaattaaaaagaaagtgtggatggaggaagggattaaaaatctggctgggcacggtggctcatgcctgtaatcccaggcgtgatttgggaggccgaggcggacagatcacgaggtcaagagattgagaccatcctggccaacatggccaaccccatctctactaaaaatacaaaaatcagtcgggcgtggtggtgcatgcctgtaatcccggctactcgggaggctgaggcaggagaatcgcttgaacctgggaggttcagtgagccaagatcgcgccactacactccagcctggcaatagagtgagactctgtctcaaaagaaaagaaaagaaaagaaaatctttggggttcttacacaaattaaatgagataatttattattattattttttttgagatggagtcttgctctgtcccccaggctggagtgcagtggtgcgatctcagctcaccgcaagctctgcctcccgggttcacgccattcccctgcctcagcctcctgagtagctgggactacaggcgcccgccaccatgcctggctaattttttgtatttttagtagagacagggtatccctgtgttagctaggatggtctcgatctcctgaccttgtgatccgcccatctcggcctcccaaagtgctgggattacaggtatgagccaccatgcccggcttgagataatttataaagtgcctaaaatacatcctagaaatattagtttttcttccttgaagtcataaattatggcttacactttttttcaggtatttctcatagtactaatgtgttgctcacactcaagggtagtagttgcttaggaagaagagaaatgtagttgaaaaagtaatagactagaagtcttgagacctgggctcatgttccaagttggcttttttttttttttttgggagatggagtctcgctcttgtcccccagcctggagtgcaatgacacgatatcgactcactgcaacctccacctcctgggttcaagtgatttctcctgcctcagcctccctagtagctgggatgacagacacccaccaccatgcctggctaatttttgtattttaagtagtgacagcattttaccatgttagccaggctggtcttgaactcctggcctcaagtgatgcgctggcctcggcctcccaaagtgctgggattacaggcatgagccactgtgcctggtcccttgctaaatgttttgttttgttttgttttgtttttgaggtggagtcttgctctgtcacccaggctggagtgcggtggcatgatctccgctcactgcaagctccgcctcccaggttcccgccattctcctgcctcagcctcccgagtagctgggactacaggcgcccgccaccacgcccggctaattttttgtatttttagtagagatggggtttcaccgtgttagccaggatggtctccatctcctgacctcgtgatgcacccacctcggcctcccaaagtgctgggattacaggcgtgagccaccgtgccccgcagttgcttgctaaatcttttaactgctggtcccattttcctcatctatgaaatatttaatggaagtgtactattaaagaaacttttctttgctgatgaatgcaggaggtatcattaaaaacccacatagtgctattttcataattactctttatgtattgtgttcttgggttgaatacttttgttctagagttacaattatttgtgtttcttaccaggtttaagaattgtttaagctgcatcaatggagcacatacagggagcttggaagacgatcagcaatggttttggattcaaagatgccgtgtttgatggctccagctgcatctctcctacaatagttcagcagtttggctatcagcgccgggcatcagatgatggcaaactcacagatccttctaagacaagcaacactatccgtgttttcttgccgaacaagcaaagaacagtggtatgtgaacattctacttaggaaatttagctatttatctgcctgtggagcacattaaggatcatgttcaacttaaagacaggcaaaatattcattgtcatttagggtctttatttttttttttctaactgcagatttatttttttatattgctgttccttccacaccccctattttttcctacctcttggccttccttctgttactcttgcctggaatgtcttcctttgtgccacttcatccaaacaaatagtacattcttatgggtatatttcaaagacttttctttgagaagtctctaggcctttccaactacttattttagaagacattttatttcttctattaaaatattcacctaaagctttttgactattacaatcaagtataaagaagaaagtaaagttacatagaaaagattatttttgtatatttcataggcccaggaccagtctggaggcagcttagaaatcatagaatcttctttttcagggcactgacaccagccacttagttctgcgtagtttattttttcagtgccagtgacaggttcatattggcatcatggcaggacactgccactaggttttctgatagaaaatttctttttctttttcttttcttttttttttttttgagacggattctcactctgtcacccaggctggagtgcagctcactgcaacctctgcctcctgggttgaagtgattctcctgcctcagcctcccaaatagctgggactacaggcacacaccgccacgcctggctgatttttgttttttgtatttttagtagagacggggtttcaccatgttaaccaggctggtctcaaactcctgacctcaggtaatccacctgcctcggcctcccaaactgctgggattaccaacatgagacaccacgcccagcctgataacaaaacttcaatttttctaagaatttagctctcaaaaagttttctggctgggtgtggtgatttatacctgtaatcccagcactttgggagaccgaggtgggcagattgcttgagctcaggagttcgagaccagtcgggcaacgtggcaaaccccatctctacaaaaaaaaattcaaaaaagtaggcctggtgcagtggcttacgcctgtaatcctagcactttgggaggctgaggccagctcattacttgaggtcaggagttcgagacaagcctggccaacatggtgaaaccccatctctactaaaattgcaaaaattacagccaggcatggtgttgcacgtttgtaatcccagctacttgggaggctgaggcaggagaatcactcgaacccgggaggcagaggttgcagtgggccaggattgcgccactgcactccagcctgggcgaaagggtgagactatattaaaaaaagaaataacaacaaaaatgtagccgggcgtggtggcacacgtctgtagtcccagctactcggtactcgggaggctgaggtgggaggatggcttgagcccaggaggcaaaggttgcagtgagctgagattgcaccacttcaccccagcctgggtgacagagagccagaccccttctcaaagaaaagaaaaacaaaaaaagttttctactattatggataaaacaaacaaaaccaaccacctggccaaaacagaaaagtgaaattgcattggttttgcttggtggaacttttgagaaaacttgggttcaaaacttccatgcctcttcctttcccatcctctgttctttgtgtaaaatcaatgcattgtgtttattccatatagtcaggtgaagcaaggttctgaggtggggaaccccagtccagagttttctgtttgcttctaacagttccactcttcccaatttgttaataaattgtttatactttttttgtgaacctaaggagcctcccaagtgtagtgttgaatacttaggtgcattttgaactgaaggcaaaactcaaaagtctaactttaattaaagtttgagtaagtttatctttgtctctcttcctaaaaatgaaaattttatggctggcaaaataagcagtaataatcccctatatctgaacaatggtcttccatttgcaaagtaattttgcctactgtttctcattaattttctttgtgaccttaaattgagaagtcagataggaagtgtgtgttatgagaagctgaagaccatttggtgcttcttcaaagtgttattgagactatcttctccatccccatctgctaccagtttgcccagaaggctgggaaacttaatttggcatagtgattaagtgtatgaacctttaaaacaagaaaatcccaatttaaatcctcattgccttttattagctgtatcatttagacaagttctgtacttttttgatcctctttcctgacctttatgaaatgaggcttgtacttagcacagtggctgattcataagtgaagtggtagctattattattattattatatgtattttttttagatggaggctctcactgtcacccaggctggagtgcagtggcccaatctcggctcactgcaacctctacctcccaggttcaagcgattctccttgcctcagcctcccaagtagctgggattgcaggcacccgccaccacgcctggctaatttgtttgtatttttagtagagacagggtttaccatgttggcaaggctggtctcaaactcctgaccttctgatccgcctgcctcgtcttcccaaagtgctgggattacagacatgagccactgcacccggctgctatgattatttcttagctttttatacatctatgtagtccttgatcccctccatttgagcacagctggtggttggaagccaagcttgacttctcctacagcttatgagaaggttgtagcctaggttagttttgcctgtttctttgggtaaagatgaactaactgtggaagaactagctgctttcaccaggcacgcagcttgaggaaagcggtagaagagggaagagttgcttagctaggccagcaccatcagtcagctctttttactcctccccaggttgctttacttcctgaacccagaatgactctcataatcactcagtgggttctagaaattatttaactgatttcagcatgtatccatggagggctgtaaagaggagaatgagacagaggacgcgtatctgatttaaataattttagatgtgataattaggtttttgaatgtttcttggaatttttattttctaaatgtgtgcctctttgacttcctcctgctgctgttgctgctattgctgctgctgctactgcttctaattattattagataagtgattgactggagccgaggaccactactattagagtcagctgaccagcaggttaaaatacagattcattctgtatgaatgggctttattccatactaactgaatcagaatccttggatgtgttggacaggtagatggaatctatattttctcaagcttctctgaggattctaatgccagctacatttgggaatctgactggattagatgatatttaaagaactgtccagcttgcagtatgatttagtgaagactgataatgtaacagatatcactttatagcttagaaaacattgctatacagtatttgatgcaggtcatgattccgttaggtatgtttattactctttgttttcctcattcttagtgtcttagtagttcacatcagtatagcttacttgttttgtttctgaaaagctggaagttggtgggtatcactgcgtcaagaaacttttaaaataaacttattttggaacattaaaaaatatatacaggctgggtgcagaggctcttccctgtaatcccagcactttgggaggctgaggtggaaggattgcttgagcccaggagtttgagaccagcctgggcaatatagtgagatcttgtctctacaaaaaaaaaaaacattagctagaagtggtgctgcccacctgtggtcccagccgaggctgaggcagggggatcacttaaactggggtggtaaagggtacatgtgtcatgatcatgccattgtattccagcctagatgacagagcaagattctgtctcagtatatataatatagattttacacacacacacacacgcacgcacgtagagaaaataacaaatctgatgtaccccttacccactttcaacacttagctaaccatggcaggcctgcttaatctgttttcatccactcctttcccagtgttttgacacaaatcccaggtatcatttgtctgaactattttggtatgtacaagaaacttttaaagaatgctaattttatttatttttaaataggtaaagcattcatatgagccaaaagtcttgggtgacccctgcccctgtatccccatttcttttcctcagaggtttcttatgatcaatctttatctattcgaagaatcagttggtttccccttaccctgttgttcacgacctttcctttcttccacatctctgaaggagaggaaaaaccatcggtagctaaggaggctatcacaaactccaaaggaacttttttcgtttggagaatcttttccttctcccagatgattgatcctcctggagaatattccttccccactcccatcaccttctcgactaatctgttacaagttcaaattcttctatactgtactctcaatgtggagtccatctttgggcttcaaagaatgattactgggcagataagtccccttcagtccctggtgatagcaaaataaagccttgtgaaaaacttcttacgttgcccctctctgatgttttcaaattccttatgcttacatgcattcccttctttcctagattgttttctctgctttgcatccacatactatgccctagtttggagcctggtaactagaagggcccagataactatgtcctctttcttaagacttttttctgttgtaaaccagctagagaaggttggctggattggcattgaggtggctggagtaagagccaagattaagaacactttgggccttttgcagccctgctttacttccttccccctccccgtgtacccacataggtagatatatgcatacactcaccaccttctgggggcggggtgtggggggggatggcggggtgggggagcggttggctggctgctgtcagctgttagcactttcaatcagaggaggaacctggtaggcagttcacaagcactgcaaatctctgttttgccctccttgctggccatactgactctagttaccttacttttgattaattcttggctttgaagttaaacatggagggcttttatcaaaactctgaaattttcattcaaatttttttacagctgccattaattgtgagtatcctgggcactcacacttcccagtagggttctgagtacctgcctagctttttgaggattgagtacaggggaatatagagaagtatgtgccactaaggctgcttggtatggtgtgcacatatatttaaactaagattggtgtttgtccctacagcagggctggagttctatatcttccacttcctgctttgccttcactagtgttaagtacttgtgagatggaatttttgttagaatcatcagtcatttttgttgaaagaggttgaagtacaaattttgatcataaaaactcgtttgtttatagatcagattggcttatttcctctctaatgaatctagtgaacatatatgtgtatacattctaatcacacaaaattagagacgtataaaggaaaagtttatcattttatctttcttaaccactttctaaccactttcttaactgtctcttgatgtgaacagctaggtgtaaatctttccacttgtataacatatacagatttcttcactttttttttttttttttttgagacggagtctcgttcttgtcacccaggctggagtgcaatggtgcgatctcagctcactgcaacctctccctcctgggttccagcaattctcctacctcagcctcccaagtagctgagattacaggcgtccaccaccatgcccggctaatttttgtatttttagtagagacggggtttcaccatgttggccaggctggtctcgtactcctgacctcaggtgatccacccgcctcggcttcccaaagtgctgagattacaggcgtgagccaccgtgcctggcctcttcacctttaaaataatcttactctattattctgaaggatattttcccccaattaatatatcatggactcctctccatccaggtcattataagtaatataatagctgcataatgtgacataatacagatgtctcacactccattcaagtactttcctattgctggacattcaggttgtttcgtatatgtgtgtgtgcgtgggccatcacaagcaatacagactggtgcatttatttctgtgcccacctttccaaggggtgctgcagcctgtgttggtcctaaaggtggtcctttgtttgtaggtcaatgtgcgaaatggaatgagcttgcatgactgccttatgaaagcactcaaggtgaggggcctgcaaccagagtgctgtgcagtgttcagacttctccacgaacacaaagggtaagagctcaaaagtcaattgacttcttcagactagtaaggatcttctagcttcaaatagctatgtttgtattaaattgtactagcttcctatagaatattgtatatttctatacctttctttataaagagataattcagaaaaataggtattaagaaattgaaattattgcttggacattctcttgaaaagttaaatacacgttaagctgggcctgatgaccaatacctgtaatttttttttctttttgaggtggagtcttgctctgtcgcccaggctggagtgcagtggcgcgatctcggctcaccgcaagctccgcctcccgggttcacgccattttcctgcctcagcctccggagtagctgggactacaggcgcccaccaccgcgcccggctaattttttgtatttttagtagagacggggtttcaccgtgttagccaggatggtaatacccgtaattttaacactgggaaactgaggcaagagggttgcttgaggccaagagttcaagaccagcctgggcacatagcgaaggcccatctctacaaaagatttttaaaaattagccaggcatggtggtgcggccctgtagtcctagctgttcgaaaggctgacgtgagaatattgcatgaccccaggggcttgaggctgcagtgagtcatgattgtgctactggactccagcctgggctggagcaagatcctgtctattaaaaaaagccaaaaaacaaaaaaacaaaaacaaacacatgttaggtattgataatgtttccatggatggaaacagtgatgttagatgctgtgtttttttgagacaaagatttttctttgtgtttactctaatgcattatatagactgggcactcagaaagtgcattattttatataaagaatgctatcctcgggagattgacttttctcattcactaatttttttttttattcagttaaatgtgtactaatccctgctgtttgatagattgtttaaagatgcagaagcatttctgcttcagggaagattcatggtttatcctattcctaatggtggtggcaagatagaggcatcccctcaaaggctaggagtaatacctcaaagcagcagagctgtccataattatccattatccattattccctccaccccgaaaatatagggaaacctttaaagggttcttttttaccctcttcttggaaagcgtcacttatgttattcatcgttagcttacattttttcatgtttcaaagagttctgcagtttgggagaatagcccagggaatgaatctactcgaaggggtgagtgtaattctcaatttaggaggcgtttgttgaagtgcaaatctttgaagcagacgttaacttttgctgaaggtagcccaggttgggtccctaagccaatccatagtgttccttaaggactagagagattctgagacagggagggcttggtctactctcatccaaggctgcactggtttggagctactctggagtctctaggacagacagcagattgtcactaggatcagtctgcagattgatgagaaaataaggcttgtcctccttctcttcataagggcaaaatagtcctttggagttataggaagttttccaggtgctgtataggtaattatattaaggaatgtattgtttactgttggatagtgagaaaaatggcttgactaggcttctggtagataatggagaggcttgaatggtgctatacatgttattttctctttacctgagaatattcttcctttggaaaatgggccagattaactggataaaacataagaaaggaattgggcattactttttacttatgtatctattttttgtcttatttatactgtaggcacagaaagtgtggtttcagagtagattttaagacagttaagtttctcattgacttatagaccctacaactacagatttgagtctgttattaattaatagaaaagtacatttttcatttgtggttcctttctatttatctagattgaaataggctactgaagactaaattttgtactgcagcaatatttataatccattttacaggatttggggatttttgtaagattttagtgttacaaattccaatttaacgtatattgactttattgtgagattttatatatcattgtttaaagaaaactttattctggccagacatggtggttcacacctgtaatcccagcactttgggaggctgaggcaggaggaccgcttgaggccagggattcaagaccagcctgggcaacacagcaagactctctctctaccaaaaaacatttttttaagtaaataaagagaaaactttattctgagaacatgggctttggagttcaacagacctagattccaacataggcccttaaacttgctgtgtggccttgagcaaattaccttcttagagtcccagttttcttatttttcagatagaaataatacctacttcataggtttgttgtatgaattaaataaattattgttgtatggattaaataaagttgtgtttatatggcatgtgataaatggtagctgttgttatttctattgaactttgatcttgtttaaacatttcatgttttttttaaatcctttctagtaaaaaagcacgcttagattggaatactgatgctgcgtctttgattggagaagaacttcaagtagatttcctggatcatgttcccctcacaacacacaactttgtaagttgcagatctcttctctttctggcatgttgagggctttgccaggcataacagagatttctcaggtaatatgcgtatgtatatatatatatagttggattgtttaaagttctttatgctgttgtttacagtaaggcaatttagatttcattagtcagagatatactctaatttgtgattatgaattctgtacatgctggaagtatgattcattttgtaaaaacttttttggaggccaagaaatgatgttgtcttttgtcatcttttatttattcagcataatttacacctgtgttcttgttgtaggctcggaagacgttcctgaagcttgccttctgtgacatctgtcagaaattcctgctcaatggatttcgatgtcagacttgtggctacaaatttcatgagcactgtagcaccaaagtacctactatgtgtgtggactggagtaacatcagacaactcttgtaaggcattgttcttttatccaaggaagatagggatgaggagtatacatactttaaagggtatttgttgtagattttgactgacaggtctggattctagactcatttaatgaattgtgatccagaaactactttagaaacagtgataattctgaaactagctaggtttggtggcattcatactccagaatgagcaggtaggagtaggacttgttatctgtcaaattgagattgacatactgtgactgtgattcagtaaggaaaggagcaaaaggatatgaaaacaagaagattttttgcttttcgctcttaatagtattatctactagggttgctagtagacactgctctgtattttgttgaatatgctgaatgagcctttgacattgagaaggagcagaaagcacggttgatgctattttcttcacttcaaactggagaaaacttagttgtttggacttaaaattgtttgaatataaaatcttgaaagattcttgtttctttcaggagacaatatatttcatatagataaaatgttattaaagaatttaaagtttacattaaaagtacatggtccaaactgccttttaaaaactgtaactaggtatatgaaaagtttaaaagttttgtccttttttgacagtactagagaaaccaagggagtgttattattagaccatgatgaaaacgtttttgctttcatggtcacttacgtattgattttgtgatgagagcttgagtagcacaaatggcacaagcttttaaaatttatcttatttttgtcccccaccctttttttttttttttttttttggagacaagtctctttctgtcattaggctggagtacagtggcatgatctcggctcactgcaacctctgcctcccaggttcaagtgattctcctgcctcagcctcccgagtagctcagactacaggcacacaccaccacgcccagctaatttttgtagttttagtagagatgaggtttcaccatcttggccaggatggtctcgatctcttgacctcatgatctgcccacctcggcctcccaaagtgctgggattacaggcatgagccaccacgcccagcctttttttttattattattttttaaagacagggtctcgctctgtctcccacagtggagtgcagtggcatgatcacagctcactgtagcctcgacctctcgggttcaagtaatcctcctacctcagcctcctgagtagctgggactacaagtgtatgccatcatgcctagctaatttttgtattttttctagagacggggtttcagcatgttgcccaggctggtctcgaactcctgagctcaagcaatctgtccgccttggccttccaaagtgttgtggttacaggtgtgagccaccgcatccggcggcacaagcttttgagtctaacagacatatgtcaaaatctcagtgttgtcattaaccataaccacatctgagttttcgttcatgcatctgaataaaggggatattaccttccttgcattgttcttatgaggcttgctgacataacctgtgaaattactaagcacaagtgcccacctcatggaaaaaaggtgcctaattactcacttttctgtgatttattccttctattttagtcttttatctatgcattttcaagatggaatgtttccagagaagctgtgtgtgacatagtttgtgaaatgttatactgtagtttgaaaaatattattttgatatagctagacacaggaccagtatttcctagaaatgcacactgggccgggcgcggtggctcacgcctgtaatcccagcactttgggaggccaaggcaggtgaatcacctgaggtcaggagttcgagaccagcctggccaacatagtgaaaccccgtctctgctaaaaatacaaaaattgggggaagggatagcattaggagagacacctaatgttaaatgacaagttactgggtgcagcacaccaacatggcacatgtatacctatgtaacaaacctgcatgttgtgcacatgtaccctaaaacttaaagtataattaaaaaaaaaatacgaaaattagctgggcatggtggtgtgtgcctgtaatcccagctactcgggaggctgaggcaggagaacccgggaggtggaggttgcagtgagccgccattacacctctgcactccagcctgggcaacagagtgagactccatcttaaaaaaaaagaaaaagaaaaagcacacaggagcctgtatgtttattggcaggtcagtattattcacattcaataatcattcaaatccagttatttggaatattgttccctttattctaggtaatgtaaaacagttgaggaaaatgtgactgggaaaagttcagttttagtagctctgagtttgcaaaagcaaggcatgctgattgtctctgtaagattactgcaagcctaaaaaccagtctttccctgcttttgtttagattgtttccaaattccactattggtgatagtggagtcccagcactaccttctttgactatgcgtcgtatgcgagagtctgtttccaggatgcctgttaggtaattttttacctatagcttttcttttagaaagttatttggggtggtggggttggaagcttgaagacaaaaaataagagtttcttcgcattccctcctctctacgtggaaaccccttgctgcttctgtggaacttgatactggtggtacagcaaaaggtagaaatttctgtttatggacctgtaggtcttacattctggaaagtgactttgactgtagcttcttctgttatcatagcatatttcttaatatgtcattacattttaaagagcttgagattctgctttcctcagtatgtactgagttcaacctcaatggaaagggtcctaaaacttaatacagtgatttgataaaaataaaacccttaactttgaaatgcatgttgtggccgatgcatttgctaaaaccatgtatttaaatagactagtgtctttaaaaacatttaattagattttcagcataaatattgtttctcatgtgtctctgagtttgcatataacttgtctttctttactctgttttccagctttataatcagttttgttgcgtttatctactgctcagtgttaacacacatgaatttgaaacctaaagtaaaatctacatccaaaatatcttactttaggccaggcacggtagctcacacctgtaatcccagcactttgggaggccgaggcagatggaccacttgaggtcaggagttccagactagcctggccaacatggtgaaaccccatctctactaaaaatacaaaaaactgggtgggtgtggtgatatgtgccttttggcccaggtacttgggaggctgaagcaggagaatcttgaacgtggtaggcagtgagctgagatggcaccactgcactccagccttggtgacagagcaagactctgtctccaaaaaaaaatatattatgtatacacacacacacacacacacacacacacacacacacacacacacacacataatgtgtaatcagataatgtttaatgtgaaaatactatggaaatattaaacgcagcatatcttagaataaggaatttgcatatatctggatatatatttctgtatggcttttatttttcttgataatttgaaaagcaaatctgaccaagaatttgtagttacctctgaagattagaagaaaccaggcctctgaagccataaaacagaggattatgtgggaaggcatttttttcaagacaatagaacaatttcccttagaaaagctggcctttttccctttaattcatacatgggtgttacctgaatctgaacaaacctcgaacgaatctttagagcaaataatgaaaatgttatacctcttaatgcatgttcccagtttggttggtggggttggtggtgactggaagaggccagtggttaatttcacatttaggtatttccatctaaaaactgaattcccatttatttactttgtttgctggttgtagcaggtaaggacaaacagagggtaaaatcctggcctttttacagacatgctcagcacgtctacttatctgtttaaataaattctcaaatttagtctctaaactgggcgtgttccaactagcttaataggtggtagcgtggttgtcaaatgttaatctgttctttcctggagatgttgtaaaaatttggagtagagtggtgctttatttaaaaaaagaaaacttataatgcactctccttttcattgaattcccaatacatgtattatttcctgttccaaattttgtatgcaaaagcacctagacttaagataatttttagatgtcacacatttgaaagaatcaaacattttgtcaaaggttgtacaggtagagtttgcccttaagcatcttacttagtcaaatatgtacttgaaagacttcaccagtatgaaagcctaagtgccaatcatggaattttctttctcctcctagttctcagcacagatattctacacctcacgccttcacctttaacacctccagtccctcatctgaaggttccctctcccagaggcagaggtcgacatccacacctaatgtccacatggtcagcaccaccctgcctgtggacagcaggatgattgaggtaatagggcaccttgggggtggtaatgtcagtcaattaatggggtgaggttgatacttatttcagagttttgggtttcaaatctgatcaaggaatgttgcaacactttctcaggtctctggacttttacagtttattttatatccataatatcttcagactggctgaatagtctggttagtatatcattcaactggagaactaaaacttcctgaaaaaatgttaacatttgaactcttcccattatcagatttgaataggctattaatgaacaagtgtctaagatatttaaagagcagtttagttttggtgtgggacagaaattaacagtgatggagaactacagattctctggaagacttttgtgattttatttagaaataaaagggtggagtcctaggactttaataagcaggtgtttggggagatgtcaaagtgcccaaagctagtgtttttgaactgctttttcttctcttggctttttggttatgtcctattggtttaatttgctttctgcttcatctttaataacaactgaatacacttaaatacttcctttgttctttattcttctttatttctcattgctttggactagaataacaacctgagtgcttctcccagggcatggtccagacgattttgtttgaggggaagagtaggtatttttcttcatgcctttgctttcttgtaattaacaggattgctaaaactgtcagacagcagactaccaaaaatgaaatagttgctaagttaaatttatatttcttgtcacttgtttccatgttttctttttctttctttctttttaaaattttttttggcagtagagtatatagaagtaaaaaaaatgttgtatgtggtattgatgataggtgaaatgaatttctgaagttaggccaggcatgacggtgtatgtctgttgtcccagctactccagaggctaaggcaggaggatcactggagcccagaagttctaggctgtagggagctacaattgtgcctgtgaatagccattgcactccaactggggcaacataataagaatccaccttaaaaacaaacaaaaaatgttaagttagattttgaggccaagggcattaaaaagttttttttttaaatcaattccaaccaaaggctaatgttagacttacttagttggtgctcacagcattggtattctgtttatacattagtaaccaaatgtgtttttggttgataaaccctagaataaatattctttattgaaagcttatcagagacaactatgctctctctcatcatgtagacacctgctgcgttaggcacagtttatctcattcagacctcaaatcactttcaacataattgtcctgccactattgtgaggagatcatgtataagctataaattttattattttgactttatcattatgattagtcctgataatacaataatataccagttactgctacttctattaaatggtttgttcctgtatgaacactgtaatacttacagggaacagtaaaggtcagaattggctgggtgggaagatcacttgcgaccaggagttcaagacctacctgggctatatgtagcaaaaccccacctctacaaaaaaaaatgtaaaaattagctgggcttggtggtgtgcacctgcagtcctagttactcaggaggcttgggcaggaggattacttgagcccaggagtttgaggttgtagtgagctgtgtatgattgtgtcaagtaagaatttttgagtttttattataaaagaattagcacaattgttgtgcctaatcattttttactttagaagcagggtaaattttgattcctgttaatttaatcacatataagtcagcatttttaaagtagactaattgttgctttattcaaattatttgtgggtctcaaattattcatagttctcttgagtatttagactccaggaacaataggaaaattctttctagaataaattgatccaactatagaaattagcacagaataaaatatgggatatttaattgatacagggaagaaaattaccataacattaaggaaaatattctgctacataggaatataattgtggttaataaaaataaattgtgctttgctttaaaaacaaagaacagcttagttggataatgaaattacagctgccgatttctattgaaatccacattattttttgccagtgttttgcccacctggcagttatcctgctgtacttaaaaacacacattcctggacttctcacattcccctccaaaacatgctcagtcaatcgtgggtcggggattaggggtggatcttcattcttctttccagagtcagaatcactctccaggtaattctgaagactagctagttttgggaaccagaactaggctttcttgttaaattccgaattatgttttgggagcaggggaacagcttggtttgattctttttatctaattatataattagatatataattttatctttttatataattgagtgggagcattctagtaatagttgtgtggaacaagtatcttgtctatactgtagttacacaaagagaatatagtaggacttccccccaaaaaatgtccttttttaggatatgggggccaagtggtttcatattattctattatactgttctattccaagcgatgaattttagattggggtttaggtctcatggagccctctgcaatttaaactattttccaaacagtttctaataaattctaaagatagcctttgctttctcccatgaggagaatgtaaccgatttccaaatttacccataaggcagtgttttgtggtgaaagagctgagggctgagatccatatatgatggtttctggttctatttctgccacctactggttctgccaagtgaccctgccaagtctctctacctgttcagatgtgttttcttatatgtaaaatgtaggttttgaacttggatttgtggtctttccagctttctgtgattttaggcttggataaagtatataggctgcttacctttttcaaatccaacttctagtcaatttagcctaactccttgtggagtaagagtgagcttcccccagaatccacctccccaccctggctttttaaaaaaagttttgagcctcagtggaacaagaatcccaatctttggaagggtctcagctgagagtaactttgctagcttcccttgaaagagtatgtttgttgtgtacattgctttcttttgagaaaaagaatgtggttttcattatatatgaaaaactaataccaggcttggcacggtggctcacgcctgtaatcccagcactttgggaggccgaggcgagaggatcacctcaggtcaggagttcaagacgagcctggccaacatggcgaagccctgtctctactaaaaatgcaaaaattagccgggcgtgctggtgcacacctgtaatcccagctactcgggagactgaggcaggagaattgcttgaacctgggaggtggaatattaaatccttctaatatttaatgaaaaatcagccttggagatactggccactgatatttgctgaatttaatcaaggaacgttgattagagtatgtttaggatttctatggtttttagaggtttttataatctattttgttcttgcacatcctcctcctcttttttccctcccccagagaaaatcttttgtgtgtaggagttgaccagctttccttttctgtttcaggatgcaattcgaagtcacagcgaatcaggtacttttccatagtcatttagccaacaataatgggctttttttctttatgcggtgtatcttctgttggcttatccttgtgtggcttctgtttgtcttgtctattaagcctcaccttcagccctgtccagtagccccaacaatctgagcccaacaggctggtcacagccgaaaacccccgtgccagcacaaagagagcgggcaccagtatctgggacccaggagaaaaacaaaattgtgagtatagacaacagtacctcctgccaattagggttcagtaagaaaaacctcgttggaaattagaatacttaaacttattttgggagaagattctaataaaatacattcaatgaaggagattataaatgtcactgtcatttttggcacacttgcatcagacagtttgccagtgctataactaaaatggtatttctcaaaagacaaaaattggaagtatggttaatatgtttatctttaaaagatatggaaacagatgacatgggttgatcctttgatgccctcattatcaaaagattattaccattgcatggagtataataatgatctctacttgtttcagaggcctcgtggacagagagattcaagctattattgggaaatagaagccagtgaagtgatgctgtccactcggattgggtcaggctcttttggaactgtttataagggtaaatggcacggtaagcttggggccctccctttactaactgcagggctttggtgtgaagtcaagtttcagcccagggggccaggaggaggagaggactgagtgctcctgggcttatagcagtactctcccttacatacttgattatacctgaagattgaacttaattctttttagactaagttcttataaagctcccaggataattagaaattagtgaataagacttgagccctataatcaaatgtcaggagtacttctcctttaaactgattaaatacagtctgcacatgggtcatgcttggaagctccttaagtgagcaagagtctgctgctatggagggagcatgggttctagaaactttaagctggaaaggaccttagagattgaaatggggactgatttgcccatggtcatgcagttaggcataggaaagctggaaatctcctgaagtaacttctctttgtcctgccctaggattagctgtgggtgtccctatcaaacagggaaggcattgacttaattcttgaatctatgtggaatattaatgttctgattttaatggaaacactttgtcacttggaagaaaggtactatttaacttatgtagttacagcttgtgtattttggcaacactgaacattttggcaacatacttagcatttctctgttaggtttttaatgcctctggctttaggactttgggaaataataggtatttccttgaaaatgctgcatgttcccaaaaagtcatctcttctaaattcagattataataaagcaaaaatcacagagtcccttggtgcctatactactttggatgacactggaattatctttagagataaatgtgcaaagattgagagaagttaaaagcatcaaatgaatggagtattaaaattcaaggtactgaaaatatcaaaccccccccatttttaggacctgggggtttttttttttttttttttttttttttttttttttttgagatagattcttgctctgttgcccaggctggagtacagtggcacaatcacagctcactgcagcctccaactcttgggctcaaacagtcctcctgcctaagcctcccaagtagctgggaccacaggtgaatgcccagctaatttgttttaccttttgtagagacaaggtctcactatgttgcccaggctggtctccaactcctggactcaagcagtcctcttgggtctctcaaaatgctgggattacaggcatgagccactgtgcccagccttaccatgtgctcgttaatgcatggtttttaccacttgtaattaatcatctgaccaatttctagttccttaagaggattggcacccgactgaacatttgtaaagtacatgtggaatgattccttttcctttgaaaattgcatctggctgggcagggtggctcacgcctgtcatcccagcactttgggaggctgaggcaggcagaacacttgagcctaggagttcaagaacagcttgggcaacatcgtgaaaccccatctctaccaaaaattaggtagatgtgatggcactcgcctgtagtcccagctacttggaaggctgaggcaggaagattgcttgagctcaggaggcgaatgttgtagtgagctcaatacagtgagtacacactactgtactccagcctgggtgaaagggcaagaccctgtctcagaaaaaaaaaaaaaaagaaaagaaaattgcatctagtatgtactactgggctgtctcctgggtcccagagaaatgatactgttgtagaatatttatttatatgtatttagagacaagatctggctctgttgcccaggctggagtagtggcacaatcttggcttactgcagtctctgcctcctgggctcaagctagccatcctcctgcctcagcctcccaagtagctaggactacaggcacatgccaccacacccagctaatttttgtattttttgtagagatggggtttcgccatgtttcctagactggtctcgaaatcatgagctcaagcgatccgcctgcctcggcctcccaaagtactgggattgcaggtgtgagccactgtgctcagccagttgcagaatattttagatggcataaatatctccaggatttcttaggaaagaacacaagcactttgtgggatagagcacttgtgtctgagataacaaggctgctagtagttgtaggaggcagagcaatggatattgcatttattgcttctgttagcattagaacatttttatatcacattttaaaagccccagctaaaagccagcggatgaagttttaagttgtacccaagtttaattttcctctggttgcgcactttcatttggggattcataatttttcaaggcattggtacgtggtactgcttctgagctttgtcttctctcaatagagtgagctttcaaactgtgataaagattatttgttacagtgttacttccataaagactgctattagaatgtagataacttgtttttaagattctaggttttttaggccaggtgcggtggctcacgcctgtaatcccagcactttgggaggccgaggtgggtggatcacgaggtcagtagattgagaccatcctggctaacacggtgaaaccccatctctactaaaaatacaacaaattagccgggcgtgggggtgggcgcctgtagtcccagctactttggaggctgaggcaggagaatggcgtgaacccgggaggcagaacttacagtgagccgagatcgtgccactccacttcagcctgggtgacagagcgagactccgtctcaaaaaaaaaaaaagattctaggttttttaagtcagaaagtctcaaaagtcagaggagtgaggagcagtggacttttatgccatgctttcagaaagcaagctctggtctatgaatgaagaagaaaaatgagtggtccaggaaacataacttctagattgttttgtgcaatacttttttccgccatattctggttcctgtatacagtatatctgttcagtatcttaaaaattacaactgttttcatgattttgattgaagatttttttaactcagcccacccacttatggaagtaaagcagaaagggtctcaaagcaactcagaagcctcaggtgcatgatttaaaactcaacatatttatttaaagcagcatctgtcaggcccaaagctcacaacctccttttgggcattaaatttggcatcaaggctgggtgcggtggctcatgcctgtaatcccagcactttgggaggccaaggcagggagatcatttgaggtcaggagttcaagaccagcctgaccgacatggtgaaaccctgtctccactaaaaataaaaaaattagccgggtgtgatggcatgcgcctgtaatcccagctacttaggaggctaaggcaggagaattgcttgaacccaggaggcgaggttgcagtgagccaagatcataccacagcactccagcctgggcgacagaacgagactctatctcaaaaaaaaaaaaaaaagaaagaaaaattctttctctaggccaggtgtggtggttcacacctgtaatccctagcactttgggaggctgagttgggaggatcactttagcccaggagatcgagaccagcctggacaacatagtgagaccctgtctctacttaaaacacaattagctgaccatggtgctgtgtgtctgttgtccccgctactcgagaaactgaggcaggaggatcacttgagcctgggagatagaggctgcagtgagccgtgataacaccactgcactccagcctgggcaacagaacaagaccctgtgtccaaaaaaaaaaaaaagaaacttaaggagtttatattctagtggagacagtaaacaggaaaagtagaatatatagtatgctgtaattgctaaggagaaaaatggaggaaaggagatatggagtggcagtcccagttcaatgtttttaataggttggtcagggaggaatctgccaagaaagtggcatttgcatggagggcgagggtgggtggtgcagatatctagggaagcagtaacatcaagtgcaaagtggaccactcacctggcctgctccgagaactcaaggagatcttatggcttcatttagagtgagtgagaggtatactaataggagtgaggtccagtggtagggtgttttagggtcctgtaaagactatcatttgggttaaatgggatctggggttgtacgagacctttaggaggtttggcaagcctttgtttgaaaatgagtgtgatgagagagctcattatctgtctgagagcccattctaactccaggtagttcctactagtagaaaatagtttgattgggtgcagtggcccacatctataaccccaacactttaggaggctgaggtgggagaatcacttgaagtcaggaatttgagaccagcctgggcaacatgagacccttgtctctacaaaaaattttaaaaattaggtgggcgtggtgatgcacacctgtattgtagtcccagttacttgggaggctgaggtgggaggatcccttgagcccaggagtttgaggctgcagtgagccgtgatggtgctgctgcactccagcctcggtgacagagcaagagccagagtggggcgaggggagggcatggaatagttctttattagagttgaaatccgttttcctataatgtttgctcattgatcctagcagactgaatgaatccctttcatggcagtccttgggttatttatatgtaaatgaggggaatgctgcagtatagaacattccttctggatttcataagaaattgcaaataatctgttaccataactgtgttaacgagagctggctggcagatggatccctgcaagtaccatgggcactgtctttggttgaccctgttcagtcttcccatcagtgacttaatcagaggtgtgatatgtatttgcatagtagtgcggatttagaaaagcgagaagagttcaactggctaggatgatggaaaaaagaaaagatcaccttttaacagagataagtcatattcattgttccaaaaagtagaactggagggggaagatgtggcttaatgataacgtgtgtggaaactgccaaggaagttcagccgactgcaaggtcagagtaagtcactgcgtgggcttggctctgaattctgaggttatattactgattagggccagaacaggtgacaccaaagggtgggttcagtgacaactagagcatgcccagaggagagctataagaaagggaatggactacaaaacgaggtccatgaaataggaaggtccttagaagcagttccccctgagcgatcatccagcatctcccaagccacatgcctgaacccctagccctgtcctgtcccctcccgttaaaggctctgcccttttctgggtcctggagcctgggtcatagcgttggccattttcccagcacttccactcagttgactgcctcattgggtcagtttaccttcacaggatttcttatcttcatcccttctttctgagtccccacagtcaaccattcttcttgtacctttcctgagctattgcagcagattcctctctggtctccctctttctctcctacaggtgtccaaatcctaccctagagtttactaaacacagctcaggtttctctcatccccctcacctcacctttatttctgatgtgcccagtctgaaacattctctgtcctatttactaaaatccttcccttggtttatagcccatttccttcagaaaacctttctgtattctctttgaaaagagatttacttaggcacctgtagtcccagctgcttggaaggctgaggttggaagattgcttgagcccaggagtttgaggccagcctgggcaacatagtgaggccccatctctaaaaaagaaaaaaaaaaaaaaaaaggatttactcccccatcttggggaactccccttctgttccagcactcctgtcttggctccaactgtaccagaatggacacttatgcaaatgattgttgtcctcctactcagggctggttatacacgtttcccatatggtgtcccatatggatccatttttctagatagaaggtagctccaaacatagtgtggatctctcccatccagtcaacagcaccttcaccggcagcccatggcaaacacatgtgcaggttaactggatgagagccactttggaggctgctgttaaaacatggggactcgttgaaactttagatgataaaaccagagatcacagggagacagtttgggcctatcgtgaggaccatctctctaccaattttcttcccaaaaatgaaatggggagggctgggtggggtggcttacgcttgtaatcccagcactccaggaggccgaggcaggcagatcatttgaggtcaggagtttgagaccagcctgggcaacatggtgaaaccccatctccacccaaaaatacaaaaattagttgggcatggtggagcatgcctgtaatcccagctactcgggaggctgaggcaggagaatcgcttgcggaggttgcagtgagccaagattgtgccactgcattccagcctaggtaacagagcgagtctccatctcaaaaaaaaaaaaggaaggaggaaggctccaacagagaggctccagaaacacttttaaaagtggcttttggccaggcacggtggctcatgcctgtaatcccagcactttgggaggccgaggtgggaggcctcacaaggtcaggagatcgagaccatcctggctaacatggtgaaaccccgtctctactaaaaacacacacaaaaaattagccagacgtggtggcgggtgcctgtagtcccagctactcgggaggctgaggcaggagaatggcgtgaacctgggaggtggagcttgcagtgagcccagatcacaccactgcactccagcctgggtgactgagcgagactctgtctcaaaaaaaaaaaaaaaaaaaaagtggcttttaaatttatagcactctaaagtggaatggatccctggatgtgtctaaagttatatcaagaggctggttttacagtgcttgacaatcagttgtcagtgttataggtagaatagcaattggagtcagactggggttttgatcctggctgcagcgttctttgttttttggctgtatgaccttgggcaagtgactaaacttctcagcttgttgtctgtgaagataaattatttacgtcagagggcagctgtgaggattaacagagataaaagtatacacagtgccaggattcagtattattagaattactttttaatggtattgaaggcagagaagcttaatttcaatatatatctctgaatttttactagggaccatcttaggtctcactgaaatgtggattcagagttcagcctcaatagttgctaaatggcctgcttccttacaccagcaaccagccccagtcattctgtatttgccaggccattcatatgtatgcactgatttcatccccacaggacaaggttttgacctgtcacacatgacctcacctctgtggcttgccagggttggtgtgaatagtttaaccaaggctatcgaaggcctaactgtagcgatagcagttaacctatgtaacttttttgagtcatttgaattatgtagagattggacctgtaatcccagcactttgggaggccaaggtgggaggattgcttaagccctggaggtcaaggctgcaatgtgccactgcactctagcctagacaacagagtgagaccctgtctcaaaaaaaaaaaaaaaaattggaaatttgccgtatctgtgtaggtatgtgattctttggataaatgattcactgtatcttcctcaaaactaggttatttgaaagactgagatcattcaactgattgcactgactgccaactaattttgcaggagatgttgcagtaaagatcctaaaggttgtcgacccaaccccagagcaattccaggccttcaggaatgaggtggctgttctgcggtgagtagaaagctggcggtccagtccctctggagtgctggagtggggagtacaaggactgtagagttagtggactgtgccgcaggttgggacgggcaggcagttaggactcactgtggagtttctgtggttggatgctcctcccttgagagcaaagggatgtttcctttagtttatgtggttgtcaagcctttcgaagagcccctttttaggagaataccctcctctgggcacagtaaactcaatagcccaatttctgtctctgggttttggtttgaggtgggcagaaataggccctatttttacctttatttcccagaacccttttttttatagctgagttgccttattttagacttcagaacagtcagctttccaatctttcagtcactatttagacttgtaggaataagtcatataatggagacttctacaaggagtccttgtgacctccacaggagggtcatggagtgtacattgatgaaagagaatgtcctctctgtaagcaaggctggcactgaactgatggcccagtgaactaatggtgggcttctgtttgctcagaatgccacccgggttatcagccgtgccatgtgtttgtttttgggactgggggtggtgttgggactgggggtggtgtcgacagcacagaacccactgtccacgggaaagcacagtagacctccctgagcactttcctcctccctctcctctcttcccctcccctccccagcaaaacacggcatgtgaacattctgcttttcatggggtacatgacaaaggacaacctggcaattgtgacccagtggtgcgagggcagcagcctctacaaacacctgcatgtccaggagaccaagtttcagatgttccagctaattgacattgcccggcagacggctcagggaatggagtgagtagatggtctgatgcctctctgggacccaggcatcaaatttgtccctaaattggaaccaggatcaggaaaagccttctagtccattaagcgattctgtgatatctttgcacaagcctctggcctgggctggaggggccaattatcaggaatgagttgttcaggttccagctgggtggggtggctcacacctgtaatcccagcactttgggaggccaaggccagtggatcacttgaggccagtagttttgagaccagccttgccaatatggcaaaaccctgtttctactgaaaatacaagaatgaaccaggcctggtggcacatgcctataatcccagctactcaggagctgggacaggagaatcgcttgaacatggaaggcagaggttgcggtgagctaagatcacgttactgcactccagcctgggctgcagagcgagactctgtctcaaaaaaaaaaaagagaagttcaggttcctccttgggactgaacttcccccttggggctcagatttgggctctgcctgctaccctggctttatcagaaacctgagaatatagtggggtgcatgtaccttctgcttggacagctgtggcaatgccttctgctcagctgtctgaggcatggctgtcccacatgagggtttaagcagatgttgtttttgggataattttttttttttaattaaaaactttttcctggccaggcacggtggctcatgcccataatcccagcactttgggaggctgaggcgggtggatgacgaggccaggagttcgaaaccagcctggccaatgtggtgaaatctcatctctactaaaaatacaaaaattagctggttgtggtggcaggcgcttgtaatcccagctactcgggaggctgaggcagaagaatcacttcaacccgggaggcggaggttgcagtgagtggagattgtgccattgcactctagcctgggtgacagagccagactccatctgaaaaaaaaaaaaaaacccaaaaaaaccacacttttttccttagagacacaggttctcactctgtcacctatgctagagtgcagcggcgcaatcatagctcactgcatccttgaactcctgggctccagctatcctcttggctcagtctcataggttgctgggactgcaggcacatgctaccgtgcccagctaattttcgtgtattttgtagagtcggaggtctcactatgttgcccaggctggtctcaaactggactcaagtgatcctcccacctttcctggctagcctagggtagtgcttctcaaacttctcctctgaagtagaggagctcctcgtacccctagacatctgggagttactaagctatagctgtgcttgcaagtcctacataaattctcacactgtctttaaaattcatatggaagttgccttctgtgtattttaagaaatggaatgacttttcagaaaaattgagatataattcatacatcataaaattcccccttttaaaatgtacacctacctcagtgtttttctggtattgagttgtgcagccaccaccactatctaattttagaacattttcattatcccggaaagaaacacatgcccattgtattagtctgtttgggttgctctaaaggaagacctaagggtgggtaatttataaagaaaagaggtttatttgactcggggttctgcagactgtacaaaaagcatgacaccagcatctgtgtctggtgaggccctcaggaagctttcactcatggcagaaggcaaggggagccacgtgtgatgtggtgagagaaaggagcaagagagagagcatggagggaggtcccagactctttaataaccaggtttcatgtgagctaatagtgtgtgaactcactcgttactgcagggaggccaccgagccgtttgtgaggaatccatccccatgacccaaacacctgccacttaggtcccacctccaacactggggatcacatttcaacttgagatttggagtggacagatatccaaacaatatacccattagaggttacccaatacctcccacccacttgcagtctactttctgtttctatggattttgcctactttatagttcaatataaatggaatcatgtaagatataatatagtcaggtaacatataatgatgtttcggtcaatgaccacatataggaaggtggtcccacaagattataatactgtatttttactgtgccttttctatgtttggctatgtttagagacacaaatactcaccatgttacaaccagctacagtattcagtacactgagggccatacagttttgtagcctaagtgcaacacgttataccatttagccagggtgtgtagaaggctgtaccttcttggtttgtgtgaatacactttatgatgtgtgcatgatgacaagttggctaacaacacatttctcagaaggtatccctgccgttaagtgatgcttggctgtatataatacataagatatggtattgtgtgcctgggctcttagacctagcatagtattttcaaggttaatgtgtagcatgagtcactacttcattcctttctgtgtctgagtaacattccattgtatggatatgccacattattcattcatcatttatggacattgggttatcagaattactttagagtaaaactgatgcttgaagaagtgtcagcaatggtcaggcgccaggggcccatgcctgtaatccaagcattttggaggccaacatgggaggatcacttgagcccaggagtcaagaccagcttgggcaacagtgcaagaccctgtatctacccaaaaaaaaaaaaaaaaaaaaaaaggcggcatggtggcacatgcctgtggtcccggctactgggaggtgggaggatcacttgagcccaggaggttaaggctgcagtaagctattgactgcactccagtctccaaaaaaaaaaaaaaaaaggtgtaagcatgtttgtgctgtggcctcaccttcaggtaagcagtgatgtgaaccaggctgaacagcacagggtctatccctgtgtgtaacactccttggagccaggccttcagtggctttacttcttagctgtagtttaaaactgctttctactcatgcccctcaaacttatttttaataatttcttttcccttcacagctatttgcatgcaaagaacatcatccatagagacatgaaatccaacagtatcctttggttgttgagttcatttgactgctcggttctaaatttagggaaacagaagggaggctttctatcacaagtggctctcggtgccaggggatatctttttaaggaaagaggcagaggacaggaaaacagaaaagtcagaaaattagtaggcttggcctgtccctcagcagcttatgcctcacctggactgatgagagcgatgtttaggttaggttcctttctgagtttatctcagcaaaagtgatttggagagatttccgtaagcttgaaataggcataattttatcacactattagtaaatgtaacctgacggggattgggcttttgtcttaagtttatttctagtttgtggccagcgtgtgtatgtctatctgcttgttatgtggatagcaagtagctacaagccaaatgttgaaaggtttccaaaatcactaattaaaatagtctttcttgactgggcgtgatggctcacacctataatcccagcactttgggaggctgaggcaggtggatcacttgaggctaggagtttgagacttgcctggccaatgtggtgaaaccccatctctaaatttaaaaattagctgagtgtggtggcacgtacctataatcccagctactcaggaggctgaggcacgagaattgcttgaacctgggaggcagaggttgcagtgagctgagatcacgccactgcactccagccttggggacagagcaaggctgtgtctcaaaaaaataaataaataaaatggtctttctcaaaggtacataagtgggttcttcagaagtcactattagaagaggagaggggttgtttttatagaagagtaaatgaagaaaggtatttttaatgctgtgaggcgtgaaatttaacaattttgaatctgccaccctccacgagcctttccttgtgaaagaaagatggcattacaacccacgttttgcctcttgagcagtgagaggcatgatagttgtgttggattatgggacatggcctattttaggtacatgtctgaggtgtggaacacctttcagtggtggggtttttagcagccaaacattataccatgaaagcagacaccacagatttaaggaggtgtgaattcctgggcaccaacatcacaagttactttgtgtgtgttttgttttttaattttttgttcttttttaatttttttttcctcacaagtttgacttaaactgtatgacttctttacccagaagcgagccgacttcagttctcattttgaagtcactgagtggtaccgattctagtgaggaatttcttactacaacattgaacactcagtaagggatttgctattttgttaaccactcaagtttcagatggtgatttgagggcagaatacaggcagaaacgactgtaagctgtcaggccatccttggccctctggggagcactggagtgtggcctctgctcatcctgttagggtttcaagtacctgtattatgtggaaaggtcacaaggccagagacccagcacctagatgtgcaaatggggagaagaagcagggaagaaacgctggcttgcttttggctagggccaaataatctggcacattgaccaatccctgcctgtcttctggaagaaggtgcatttcaaaagcactttaaagaacttcagaaaccttaggaagttcagtgcagagaggctgtgacagaggtaaggtggagagattaccgtgttataaagaactttgggatatttttcaaaattaacctgaccattcttttgaaaccagagtccttaacaagcattgagatatatttctccatgaaggcttaacagtgaaaattggagattttggtttggcaacagtaaagtcacgctggagtggttctcagcaggttgaacaacctactggctctgtcctctggatggtgagaatctgggctcccaccagcagtctctggtatagggcaaaaggaatgccttggagatttatgtgcaaacttaaagcgtttctgtacatttccccgaaatccacatgacccctagtgacagccagcctcagggcaattgtagattttcttgaggaagctgttgatcagaaccactgtgaagcttagtgtggagaggagttaataagctgggtgacagaaatgctgggtcttggtcctttaaagacaaggattcctgagctgttttaaccagtgcctgagttggagtcctttgggggaaaagctatgtggggactgaagaatggactcattcataactaatgaaagggacagcctggcccctagatgtctgtgaggcctgtcatatggtgataaatgcacttttgtcatatggtgatacatgtaggccccagaggtgatccgaatgcaggataacaacccattcagtttccagtcggatgtctactcctatggcatcgtattgtatgaactgatgacgggggagcttccttattctcacatcaacaaccgagatcaggtaagtctgtgctggtgcgaaaggacccaactcgtgggagcccctgggcctccgccagcctaagcagctagagggttaggacttgttattatctgttgttcattcaccccccattagctcagctgttttctttcccttagatcatcttcatggtgggccgaggatatgcctccccagatcttagtaagctatataagaactgccccaaagcaatgaagaggctggtagctgactgtgtgaagaaagtaaaggaagagaggcctctttttccccaggtaaggctcagggctgctagaatgtgattaaagcatgggttggttcgtaaagatggcaatataaggtgggagtgttttgttttgttttatagggaggggacccaggtcctctacaagatggtggggggcagggtacatcctgtgtctttgagacacagctaatgagagcattcttgggctttgtttcagatcctgtcttccattgagctgctccaacactctctaccgaagatcaaccggagcgcttccgagccatccttgcatcgggcagcccacactgaggatatcaatgcttgcacgctgaccacgtccccgaggctgcctgtcttctagttgactttgcacctgtcttcaggctgccaggggaggaggagaagccagcaggcaccacttttctgctccctttctccagaggcagaacacatgttttcagagaagctgctgctaaggaccttctagactgctcacagggccttaacttcatgttgccttcttttctatccctttgggccctgggagaaggaagccatttgcagtgctggtgtgtcctgctccctccccacattccccatgctcaaggcccagccttctgtagatgcgcaagtggatgttgatggtagtacaaaaagcaggggcccagccccagctgttggctacatgagtatttagaggaagtaaggtagcaggcagtccagccctgatgtggagacacatgggattttggaaatcagcttctggaggaatgcatgtcacaggcgggactttcttcagagagtggtgcagcgccagacattttgcacataaggcaccaaacagcccaggactgccgagactctggccgcccgaaggagcctgctttggtactatggaacttttcttaggggacacgtcctcctttcacagcttctaaggtgtccagtgcattgggatggttttccaggcaaggcactcggccaatccgcatctcagccctctcagggagcagtcttccatcatgctgaattttgtcttccaggagctgcccctatggggcggggccgcagggccagccttgtttctctaacaaacaaacaaacaaacagccttgtttctctagtcacatcatgtgtatacaaggaagccaggaatacaggttttcttgatgatttgggttttaattttgtttttattgcacctgacaaaatacagttatctgatggtccctcaattatgttattttaataaaataaattaaatttaggtgtaatggctggctgttacctccttttaaagtaattctgagctcacaacttgaatgccccatttgttcaccctcttcaggagcagaattcaagaacaggaaatgtgcccagagcctaggctgggaatgaatttgtaatttaacctttgtactctttgtaaacctctactgaagagttaagtataaaaattaattaagcagaaagtactctaaactcagctaataccttaagtaatacattttataaactatttatttatttggtaggtacagcttttttaaacacaaaaatagattagataaattccagcttggaacaagctagtgctggttcacaaggttatgctcacccttcaattaaaatcaaaatgactacaagacttgccatcagctctcttcaggaccactgctgggtcagaatcagaaaccttgggtgccatgaaatttttacaaaatttcaaatcaaagccaggctttgcagctagataatagatcacttgagtacgaaccacacatgtaagtgcacgtatatttgagttctcaatacaattaccctgatgggcaagaacccacaggtgagagcagaggcttggttcccctagagggccctggctggaggccccaacaccaaccagacgacaggagggccagactgctacccagtactgtacctcctgctccttcaagagcctccctaagggagaagaagatctatacttccactttgtttgctgcacatgtggcaacaagattgctaccctgatttgggacacttgagagaacttgaaaaaaatgaccacccttaaagccctagaaaaaagttgtatgtttgttaacagctatgctgcgctcactttgcattgtgtgttcttgaaagctctgtataaatcaaaattttgacgacacactaaatacactagagaaatacactatagaggaatccttttatagggctgaagactcctttggtaagaaaaatatgctgcattaggggcagctgcaagtttactatttctggggaagaaaagatcaaaggtaagagccaggtttgttttttaaagcaatcaatccaaacagtttgggtgtttgttagttgttacccctgaggggcttgaggtgtaactatatcagctataaaaatagcaattccatacatttaattaggttactttatatctttcactcttccccatggctgtaataatggagattgaatgagactaaggctaagcccaactccactcaaatccaagtcacacgtcaccttggctgcagtacagggaagctccgcacaccctggcttgggaaagtttcggccgatggagcccaagatgcagggcaaccatctactctttagggttctgatgattccactccagaaaggtgcatgaagaggtccccgagctctgtcatgtcgacatcttcattgttggggacatgccggctttctcggttctcgatgaaatcccagagccgcactgaattaaagaactgcaaaaacagccagtggacatgcctggttactgctaagagcaacaggaaggctgcgttccttgatcgttcttttgcctaccccatttctctgccaggaacggtacctggaaatgcccacagctgctaagtgtccccaactagagatggctaaagtccttaccctcacagtgccttgagaactgagctgtttccgaggtttctcaggctctgctagccgcccatcggggtaagcatggcgataaagacatttgcttccaaatgggcaggtccccttgccttgctcaaagtatttacaggcttttttcctgaaaagcagaaagaaaaagtcaagaggctggtgggaaaatgaggggtccaaactgggccactgcctgcctccatccttaccacccttacgccagaggtaggtcagcctcacattctaggtggggcagctgaggctgggagaggttgagtgatttgtctcaggtcacacacagctgggattctgatttccaagcaggataggaagtatccccacttacctgcagccttgcaaaggatattaacctgcctggggacttgctgtgtggagactgcagtgctccacaggccctccggcagctccagagcacctcctgggtcaccacagagctaagccaggcctggtcactcctctgggcactaagctctgaagctgggccacttgtctctgctcaaagttccctaggtaccccaccaagccaactctccccttcctcctggccgcagtgctgtcaaggtggctacagggaaagcagagggttttagcaactgcctaaagccataggtctccttccagttttcctgtctccaacctcggcaccagggagggcttcttcacgccttatgtgctttggaccccttctctgaacagtgtttttcaatgcataaaacatgggtctacagcatacagtgaccaaacagttcaaaatgttttccctctctcttaattctaccattctccccacagacctctatgttaagaaccctgcccaaggaaactcaatcaaatgaattcccattttgctcaaatgaactctggtttatccaatcataaaggatcccaaactgaagttaaaaaaaaaaagatacccaagaatccagagggccatctcggagtacagaggaaggggaaagtcacagaaataaagccaaacaacagaaagggcacgctgctgtcaggggcagctggggtgtgtgacagcgggagacaagaacagggaaaggaggctcctgaatccagtggttttccgtcttgtcagatgggatggccgcaggccggtggtgaagttctctgaggacggcttcatagcagcataaagaaaagccctctggccgggtgtggtggctcacacctgtaattccagcattttgggaggccaaggtgggtggatcacttgaggtcaggagtttgagaccagcctggccaacacagcgaaaccccatctctactaaaaatacacaaatgagctgggtgtggtggctggcacctgtaatcccagctactcgggaggctgaggctgaggcaggagaattgcttgaacccaggaggtggaggctgcagtgagccaagattgtgccactgcactccagcctgggagacagagtgagacttcgtctcactgggggtggtggcgggggggtagggtggggggagagagaacaagctccctggccagctgatctgatttgagcacaggtggctggagagcaggtgtgtggacgacacatcctccaggcccctgcttgcctggagttctgagcggacttcaaatgaccgtgagcagtttgctctcaccagagcctgctggacaactccagagcatcctagcacactggctatgaactcgatcaggtcaaacacattatatactggtcccccactctcacaagaatattactctttttcctcccccaggcttatctggtcaccaaggccaaaagcctccagttcactctgactcactctgtgtcctcggctctcacacccaactgtgttcattctgtttacaaatcacttcccaatctccccttcctttgggttcccacacttgtggaagcctctggggcctgcctgccagggccacttcctcactggcctccctcccactcccaccagtttccaccttcagagcagcacggaggagcttcccaaccttttttttttctttaaagagatgaggtctccctatgtctcccctggacttaagcaatctgccctcctcagcctcccaaagtgctgggattacaggcataagccactgcgcctggccccaacctgttcttaaagaccatggtcacactgggattcaagtgtcccttagattccagtctgtaggtcccaccacatccttctacacacctgtttcaatgccaggagccactcccagtgtcccccagacacaaaacctacacccttctgtgcccatgtccttccatcacttccccctacagacaggtgcttcctgcttcatggttcaggctcccatgctgcttcccctggcagcccccggtggatccaagtgctttctctgttgtgatagatggtccctcatgaagaactggtcaccagcaaacctgtatcataattgcccttttgcagtttcccatgaagttgtcttacttggcggggcacagtggctcacacctataatcctagcactttgggaagctgaggtgggtagatcatctgaggccaggagttcaagaccagcctggccaacatggcgaaaccccatccctactaaaaaaatacaaaaattagctgggtgtcgtggcgcacacctgtaatcccagctactcgggaggctgaggcagaagaatcacttgaaccctggaggcggaggttgcagtgagctgaaatcatgccactgccagcctgggtgacagagcgagactcgaaagaaaaaagaaattgtcttactaatctctacatcccccagtggtgcttagctagaaggtacctgacccatagtgagtactcagtaaatgtttgtggattgcaaaaaacacagtcattaaaggaaagcaaagcaaggaaagatccaaatagcaataacaatctccagactgcttttcagcagagcccctttctacaggctgggacccttttctacaggctggggcccttttctacaagctgggacccctctgcttgccacgccttgccctcttgtggacacacaggaagattgtatgaggaaaaaatggtaaaaaaaaaaaaaaaaaaaaaatcaagctttagtaaactaatatgcaacataaaggaaccattaaaaaaggtaatgcatagtttcacttttagtatgacaagtaaacgcctgccatacccaaccctcctgcagataagtcttaacacaaatatttcaagaagacctgaaggcaccagagaatgaacaaacgcagttagattctttggaggagtaaacacaaagaagaatagcaatggcaaaggctaagttaccttttttaaaaaaggtagcttttgtggctcacatctgtaatcccagcattttgggaggccgaggcaggtggattgcctgagctcaggagttcaagaccagcctgggaaacacagtgaaaccctgtctctactaaaatacaaaaattagccaggcgtggcggcatgcgcctgtagtcccaccttcttgggaggctgaggcagaagtgcttgaacctggaaggcggaggtggcagtgagctgagactgtgccactgcactccagcctgggctacaaagcaagactccatctccaaaaaaaaaaaaaaaaaaaaaaaaagaagtagctcttatcctggagcaggccaaaatcataaccacatggggtggctaaaactccaaggggaaatccaatctttctggcctgaagaactaaaagacaagagttcaaggaaatcacagccattggaaagtgaggaagcaatcccacaaagtaaggggcctgtgaaaaagtgctcaaaggctgtgtataaactctgcccaaatctgactaactcccaaaccacacaggaatgcgacaaagtcagctacgaatgcaaaaccagaactgagatctgaactgctacctgggtttgagttcaaacaatttacctgcctgttaaaaacagcaacacttggccaggcgcagtggctcatgcctgtaatcccagcactttgggaggccgaggtgggcggatcacctgaggtcaggagtttgagaccagccaggctaacatggtgaaaccccgtttctactaaaaatacaaaaaattggccaggtgcagtggtgcatgcctgtaatccctgctactcgggaggctgaggcaggagaatcgcttgaacccgagaggcagaggttgcagtgagccgagattgtgccactgcactccagcttgggcaacaagagtgaaactccgtctcaaaaaaaaaaaaatcatcactttacagataaaccataacagaatcctaagtctctctacaatgtaatatttacaatgtcaaggataaaatctaaaattactagacatacgaagaatcaggaaaatgtgatccattcttaaaagacaacagaggtcaacttcaagataacaaggatttcaaagtagctgctacaactatgttcaaggagatgaaaagaaaaaaagattgaaaaagaatgaatatccccagagatctatgaacaatataaaaaaactatcataaacggaagtagagtcccagcaggaaaagaaaaaaacaagacagaaaaaaagttaatgaaacaatagctaaaatttcgctcatcttggggagtgacataagcgtagagaaaaaccactccactcctaggcaaaatatcaacatgctgacaaccaggataaagaggaacatttgaggccgggcacggtggctcatgcctgtaatcccagcactttgggaggccgaggcaggaggatcacttgagcctaggagttcaagaccagcctgggctacatggcgaaaccttgtctctaccaaaaaaaattagccaattagctgggcatggtggcgcacactactggtggccccagctactcaagaggctcctgcttgagcccaggaggctgaggctgcagtgagctgagattgcaccactgcactccagcctgggcaacagagtgagaccctatctcaccgaaaaaaaaaaaaaaaaaaaaaaaacaccaaaactaaacaggtccacatcaccatgtccttgctcattgctgcatcccagagcccagcatggtgcctgagagaaggaagagaggaagaggcccctaagaccacactcctgggaaggagaacgaggacacgggctgacagcagagccaggcaggcagcaggggcacgtcgaaactcaaaagcacttaccccatcccctgtttgaaagcttcaatcaactcgttctttttattctgatcttccacccaatacacacttggaattacaaactctgatatcacacggcattctggacaagacctgaaataagaattagattactaagggagaagtcatgttacgaagcctgggcacactctctgctaaacctcttgctcaactgcctcaccactacaggcatttccttcaccagaaattccaaaagatgaaatcacaatcatccaagaaccttatttactattaacaaaatagggtttcccaatgagaacacatggacacatgtgggggaacatcacacaccggggcctgtcggtgcaggggcaaggggagggagaacatcagcacaaacagctaatgcatgcatggctgaaaacctaggtgatgggttgagaggtgcagaaaaccaccgtggcacatggatacccatgtaacaaagctacacattctgcacatgtaccccagaacttaaagcaaaaaaaatacacacacacacacacacacacacacacacacacacacacatatatggtttcctcaacaaaaaagacagatgaaataaccctcatacattgctggtgagaatgtaaaagggtgcagccactttgaacagagtctggcagattctccaacagtttaatgtagagttattataccataaaacctagcaatcccacgcccaggtgtatacccaagagaaatgaaaacacatgcccacatgctgtgttcacaaatgttgacagcagcattattcataatagttgcaaagttaaaacagcctaaatgtccactagctgaggaatggataagggaaatgtgctgcgtccatacaatggaacatattccgccaggagaaggggactctggcacatgctacagtcgggatgaactctgacaacactatgctcagtgaaaggggccaggcacaaaaggccccaaattctatgattccatttataagaagtgtccagaataggcaactctgtagagacagaaagcggatgagtggtgagttaaattgtgggcttttatctcaatagagcagttgtttcaccacacgattttaggcaaattacttgacccccgcaccccaggcctgtttcctaatctgtaactgaggagggtctttgagggaatgagatgagcggacagatgtggagttctgaacagtagaacgcgtgcagtaacctctccacatgccagctcttcccctgtcctgctggagaatttgagactcctatgttggccacattgagcacatcccatgtgccaggcatcatgcagaatgttttacatgcattattccacttcatcctcaaataaccctattttcgtttttggtggggggaaaaaacgaagctcaaaatgattaacaggcaactgggctacaggcaggtactactggatttcaaacacaaggctaggagatgaaaacaggtcagtgccatttaacacctttttacacagatcatctttgctgagttcctccccaacacccagaaagcttggtaggaattgttgcccatcttttataggaacaggcacttaggctcaggaagagtaaatgacttgctgaagttcatgcagccaggggccagaactcaccagttactcttgagggtaacggagggcttaaaagtggacggaataaagtctcaaatcaaacattctcctagctcccacagctaagacccctggctgtacttacttaatgattgggttttcaaactgtttggcacaccgccactgccggatgcaggacaaacagtacgtgtgattgcaattggagagaatcccaaatctcctctcagaagcagaggccttctccaggatcacttccatgcagatactgcacactttgtcctggcttgcctggaaggcaaaggccttttccatctcgtgttcgaacgtcaacatgcagatctgaagcacagacaggaaggaaggctttggttgtgggccactgaggagtggcaggagccctaggagtagctgcagccacactgccacagctgagatcaggaaggaacactgacagcgactgctgtgagcaaaggcccaggctcccccaagtcaggacactgacatctccctcagaccacacaaaggacttcaaaccatcactggtccagccccttgctttcctaggaggtgacatggtcaccacccatttaggactgagaacacggagatccaaaaaggttaaactgcagtggagtcagagtccgtaaggactgcggccctagtcccccagctcctctgggcactgtttcccccgactctgagccatgtctacatcagagatgctgactcgtccttaccatgaggcttgaggctggcagcctagtcctatgtaagaagcaccacttctccccaagaaaatgattcaatgaattcattcattcactaggcatgccctgaattccttctatgtgctggcatttgagcaagagtgagaagacctaggcccagcccttgtaagctcagtctgtccagatctgagacaagccaacactcaccgcagagacctcacacacttgcataaagaagacagctctaaccctctgcttccctggaagacaatggagagtgtctccttgtccgctgccacatggagtcagtactaatttacctcttgattatctaggaatacgctgtccatttaaacatgccttaggccaggtgcagtggctcacacctgtaatcccattactctgggaggccatggtaagaggactgcttgatctcaggagttcaagaccagcctaagcaacatagcaagacctcacctctgaaaaataaaataatttttttttttttttgagacagagtctctctctgtcgtccaggctggagtgcggtggtgcaatctcagctcgctgcaagctccacctcctgggttcagactattctcctgcctcagcctcccaagtagctgggactacaggcgcccgccaccacacccagttaactttttgtatttttagtagagacggggtttcaccatgttagccaggatggtctcaatctcctgaccttgtgatccgcctgcctcgtcctcccaaagtgctgggattacaggcgtgagccactgtgcccagccaaaataaaataaaattttaaaagttagccaagccaccacacctggcatttttaaatttttttattattatttttcttgagacagggtctcactcttattgcccaggctgtagtgcagtggcacaatcttggctcactgcaaccttctgcttcccaggctagggtgatcctcccacctcagtccttgctgagtagctgggactacaggtgtgcaccaccacacctggataatgtttgtatttttttttttttgtagagacggggatcttaccatgttgcccaggctggtctcaaactcctggggtcaagcaatctgcctgccttggcctgccaaagtgctgggattacaggtgtgagtcaccatgcctggccctcccccgcctcttatccagcttcgtaccctctgcatcatgcatagggcctagcataaggcaaagcctcccaaaacactgcagacattaatgactttaaaaggcccttccaacaagtggctcctcagattctatgatttgggctcaaatcttccaaaacttcacctagctggatcccaaccatgaggaagtgtgaacccagggagggaggatgctgtatctgcatgtgatagagacatacacacagacgacataccatggtcctgagctagatctgttctttgaacttagcatgattttatatttcagatacgacttctttttctctttattctgagtaattaaaaattggcaaaataggccgggcatggtggctcatgcctgtaatcccagcactttgggaggccaaggcaggcggacaacttgaggtcaggagttcgagaccagcctggccaacgtggtgaaaccccatctttcctaaaaatacaaaaagtagccgggtgtggtggtgggcgcctgtaatcccagctactcggtggggctgaggcaggaggatcacctgagccagggaagcagaggttgcagtgagccgagattgcaccactgtactccagcctgggtgagagggagactccattaaaaaaaaaaaatggcaaaatgactgcaggaaaaagaacctgaaaacaggatgtaaatataccagatacataatatgggtattactggcagggggatgcctgtggaaataccaacaattctgtcacttagtacatttcagatttcttgagtgtatatggatggccttccgtgtcctgttcagtacatttcagatttcttaacatgagttcatgcaaaggtttgtgactttaccttttcatgagccttcctctgctctgggtcgaatgggtgcaagacttgcagcctacagatttcacacacctccccgtgcaggtagacacaggcatccccaaaccggcactccccagcagctgcgtaggggcacagctgctgctcgttgctgtaggagctgctggcctccacgtcatcaaggccactcctgatggcatccaggtaggaatgcggcttcatctcggggctgggctgggggtcgctgcagctgcctggattactcaccatgctcggctgggtcttcctttcagccatgccagagagatctgaaaacaacacacagcacacatgcacatgaaaatggccccatttttctggtatgccgttagccaaagcctaacatattaagctactctgacctaagaattccactcttgagaacatatggcaaaaaaaacacaccgaaggggaaaaataaaaggaatagtataaaatgttcatagtgacattacttataattacttaaaaaaacaaaaaacaaaaaacaaaaatggcaagacagggaaatagcaaaacaataggatatacttatacaaaagaacactaaagccgtttaaaatggcaaatatggaaatgttaacacatggaccagttaacactaaaacaaacaaaaaaacagtgagagcactagaacacagtgtttccttcataccagcaaaggtaaaatcattttaagtgaactagaatagatgtctagagcaaatctgtgggtcctgaggccagtgtaaactgccccatggtcaccccttctcacgccaggagctgctgccttcccactgcacacctccgagcttgtttagaggtcacgtattctcacaggattactcatttgctgaggacagaatctgcctacctcatggtagccaattccaagaactagaaatctcttcacctggtgaggaccagccaataaaaacaacttttatggccaggtgcagtggctcttgcctgtaatcccagcactttgggaggccgaggcgggcggatcatgagaccaagagtttgagaccagcctggccaacatagtgaaacactgtctctactaaaaatacaaaaattagctgggcatggtggtgggcacctgtattcccagctactcagaaggctgaggcaagagaatcccttgaacccgggaggtggagatcatgccactgcactgcagcctaggcgacagagcgagtctccatctccaaaaaaaaaacaaaaacaaaacaaacaaaaaacacaacaaaaaaacttttacaatttgtagctttcttcctcatcaaaccctgttttaaggcacagaccatgccccaaaccctagtgtgctattcttttcccaaaggtgcaatctaaactgtcaaacactgtcagcataaaaactaagaccattcactggcctaaaagtcaaacagtgaaacatgctctttagcaaaatatttagttttcttttttttttttttttttttgagagggagtctcactcttgtcatccaggctggagtgcagtggcgtgatctcagctcactacaatctttgcctcccgggttcaagaaattctcctgcctcagcctcccaagtagctgggattacagacacctgccactacacccggctaatttttgtatttttagtagagacagggtttctactaaaccatgttggccaggctggtctcgaactcctgacctcaggtgatccacccgcctcagcctcccaaagtgttggggttataggtgtgagccactttacccagacaaaatatttacgtttgaaatgaagagcttgatgcttctcaccagctggacaaccaccatatgaagatggaagttacatgttgacaaaaagattgtgtttttatgttttaccaggccagtcttggagctcaacccagccttgggcacgagggcgaatgtatcatttgaatgatctgcagccacatggagcctcttcagaacagttctgaagtctctccgcgcagacaatctggaggtgtattaggttaggagtcctcttgacaagaggaggctagaaaggagcaccaagcattaacaagagtgctatgcaaaaagacgcaacaaaaggcttccgcttactcacttcggtctctaagaaccaatgttctcttttcacgctttccgggttcatgtgagttagttttcacaatggatgcagtgacctcggaaggagggtgaggactgtggaaagctggggagggcacactgtgggccatggtgcccacagcacctccagctgcagcagagggcctcgtgtggtcatatctaaacaaaacacacagcagatgcattacagacatgccacccacacacatctcccacactccccagatgccaatggccctccttctgctgcacttgttgaggtgaagaaggcggccatcttttccaatatttaaactctaacaggattatcgattattaacagatgcttggcaattcattgtgagggggacatttgctatctatcacctatgcttaagtgtctctgtgggacttgagtgggacagacacacagcaccagaccacacagagaacctgaaagttccaaacagatgttgagctaaaatctcctgatgcctgactgacccagtattcttttgagcaggagtccccaaaatgctgacaagggccaatgatctctccctgactgtctctcttggcactcattgacaggggaagaccaacgtgggcctacttccatcatctcccactgcactgcagagaaaaaggagggaaggaagctttgcagtctagaaagaaaagatcggctctttgctacaaaagcatgaacaagtttccggaattgtgtacaattttataactatatattcataagaataaggctgaaaagtagttttaaaaaatgaaaattaggccaggcacggtggctcacgcctgtaattccagcactttgggaagctgaggcgggaggatcacgaggtcaggagttagagaccagcctgaccaacatggtgaaaccccatctctactaaaaatacaaaaaaaaaaaaaatcagccaggcgtagtggcagatgcctgtaatctcagctacttgggaggctgaggcaggagaaccctggaggcggaggttgcagagaactgagatcgcaccactgccctccagcctgggcaacagtgagagactctatctcaaaaaaaaaaagaaaagaaaattagttttagggtactgaaactgaggttttaaacttatttgccatactttttgtgatgttgccatattacttttacattaaaatttcccccttatcaagacccattctccaaatgaaatcagtgtcacagctggaatctgttgcagagtccttgcctgcaccgagttccataggcacagtagcccttctggtagtacttgcagatggtggacggtttgctgtttgccaagtcatgtgagaataggcactgacttccttcccgacacacaccatgcataaaatacctgcagagacaagcacaggcatacaacttttagaagcacattttgctttataaaatcagcttattcttgaacttagcatacaaacatttaccaggcatatattatgtataaaggctctattaggcactggagagagatctatatatttccttgattctacaacagtgatttcagaggcactacaactgattcaatgacagcttttctgaaagaaaaacaaatgatcccatatatttctatgtgaagatatatcttcctgatttgagatatgttcaatgtgagccacataattgagaaaacactctgtgaaaaactgttctctctgttctcaaggagcttaaagggcatgactaacgaaaccagaagatctgggctcaagacccagctctgccacttaagaacatgtcacaaaaccaacttccctgggccttgctacctttccctataaaatgaagattatactacccacttaactggtcgtggtgaggatcaaataccatagtgtggtatcaaaagatatacacagatgctccttgactaatgatggagttacatcccaataaagccactgttttttgttttttgttttgagatggagtctctctgtcacccaggctgaagtgcaatggcacagtctcagctcactgcaacctctgccttccgggttcaagcgattctcctagctcagcctgggctaccttacttatgcttagaacacttacatcagcctacagttgggccaaatcatctaacacaaaatctattttacaataaagtgtagaataatctcatgcaatttattgaacaccgtactgaaagtgagaaacagaaaggttgtatgggtacttgtagtttttaatgaaagctgtttctcatacattgtttcagatgtttcagaatatcagatgtatcacatgttctgtatcagaatattccactgagatatcagatcctggccttgaagaataagtggtacaggaatacctgggaggctaactctgtccagacagggtagagagacctgagtgaatactcaaacagtagagaccctagatgaacatgtcttgatattaaagataaactaggctgggtgtggtggctcacacctgtaatcctagtactttgggaggccaaggcgggcgcatcacctgaggtcgggggttccagaccagtctgaccaacatggagaaaccccgtctctactaaaaaaaatacaaaattagtcaggagtggaggtgcatgcctgtaatcccagctacttgggaggctgaggcaggagaatcgcttgaactcaggaggtggaggttgcggtgagccgagattgcgccattgcactccagcctgggtgacaagagcaaaaactctgtctcaaaaaaaaaaaaaaaaaaaaaaagataaactagccagggcaacaaagggagaccctaaaatttaaaaattagcctagcatggtggtatgcacctgtggttcagctactcaggagagtgagacaggaggattgcttaaacccaggagttcaaggctgcagtagccatgattgtgccactgcactctagcctgggtgacagcaagatcctgtctcacaaaagaaaaaaaaaaagtaaactagtgttagagtagaagtattttagacacaccctaataaggcttaaaaaaaaaaaacacaagctgatagcaagtatataacttactgtcagccaaaacaaactttaaaggaagacaatacaatccaaatgctcagaaactcacaatgcttggcatccaatcataaattactagatatgccaaaaagcagaaaataatgtgacctataaccaggagaaaaataaagaacaggaattacagagatgatggaatcagcaaaataagaccttaagaacagctattatacatatgctcaatatgctgaaagatttaaagaaaaacataaacataatgtggagagaaatggatgatttaaataagaccaaatactaggtgtggtggctcacgcctataatcccagcgctttgggagactgaggtgggtggatgaccagaggtcaggagttcgaaaccagcctggtcaacatggtgaaacaccatctctattaaaaatacaaaaattagccaggtgtggtggcaggtgcctgtaatcccagctacttgggaggctgaggcaggagaatcacctgaaccctggaggcggaggttgcagtgagccaagatcgcgccattgcactccagcctgggcaataagagcgaaactccacctcaaaacaaaacaaaacaaaaaacaaatgaaacttctagaagtgaaaaatacaatgtctgaaatgagaattacattagatgagtttagtagatgggatactacaaaggaaaatatcagaatacttgaagacacagaatagaaaccatctgagagagagagagagagaaacaaacttgcttctgactaccaaggagcatggatcttggcttctcactgtaaaaaaacaaaacaaaacaaaacaaacccaactgaaaaatgaaacaaaatatgtgaaacaactgctttcagacaatagaaaaaaggactggtccttaagagaagggaaacacaggaagtaagccccacatttagtctgacttcctacctggaggcatattctaggtcttggtactgggagtagaacctcaggcaaatcacagagattgagtttagggaggctgcagggattctttaaagatccataaatagtctgggctcagaggctcatgcccgtaatcccaccacttcaggaggccaaggtgtgaggactgcttgaacccaggagtttgaggtcagcctgggcaacatggcaaaacccaatctgtataaaaaatacaaaaatcagccgtgcatgatggctacttgggggcctgaggtgggaggactgcttgagcccagaaggagagagcctgcagtgagctctgtttgcaccactgtactccagcctgggtgacaaagcaagaccctgtctcaaaacaaacaaaaaaacaaacaaaaaaccctgtaaatagaaccacacataggccgcatgcagtggctcatgcctgtaatcccagcactttgggaggccaaggtgagtggattgcttgagctcaggagtttgagatgagactgggcaacatggtgaaacctcgtctctaccaaaaaatatacaaaaaattagccaggcacggtagcgtgcacctgtgctcccagctacttgggaagatgaggtaggaggatcgattgagcccaggaggcagtggttgcaataagccaagatcatgctgctgcactctagcctgggtgacagagtgagaccctgtctcccaaaaaaaaaaaaaaaaaaaaaaaaaaaggtaagttggggaaagaatcttttcaacaaatgatgctggacccaaaatcgacttggaaaaaacttaaatatgtacctgctgatatgacccaaaatgaagtataaaacaacctatgacgtattctagccagaaacaattaatttgaatccacaaaactccagatctaacatccagttcatagaaaatacaggagactggggacaacctatgaaagacatctcgagaaaacaaccaaataaatacaaaagaggctgtacgtgggacctaggcctactgtctttataagtgccatgtaactaaaggaggctgagtttggaagacagtttgacagtttcttaaaaaatgtaaacataaatctaccatatgacccaacaattctacgcctaggtatgtacccaagaaaatgaaaatctatgtccacacaaatacttgtacatgaatgtccaaagcagcactatgcataacagccaaaaagtggaaacaatccaaatgtccatcaactgatgaacagacagagaaaatgtgatttatccatacaatgggctcttatccagccataaaaaggaaagaagtactggcacacactacaacatgggtgaaccttgaaaacattacgcagagtgaaagaagctggacacaaaagaccacatgttgcatgattccatttatatgcaatgtcagaaaaggcaaatctacagagacaaaaagtagattaagtggttgcctagggttgggaggagagaagtgagggtgactgttaatgggcacaagggatcttttgggggtgatagaaatgtcctaaaatttaactgtggtgatggttgtacaactttgtaaattcattaaaaagttttgcactgtacacttcaaacaggtaaattttatggtatataagttatacctcagaaaaagctgttaaaaaagagaaaaaagggaagggacaatgctaggttagcagacagaacacaagggacataaccagatgcaatacttacctctggactagaatctggtttcaacaaaccagatacaaaagacatttttgaaaccagatattttgaaagatattttgaaataaatgtgagtatggactaggtataaaatgaaaatttattaatatggaaagttaaacacaattaactgagaagagtagattataaaacagctaatggtgcagcttctatagaaaaacagtacggaagttccttaaaaaattaaaaatatatttaccatatgatccggcaattccacttctgggtatagacacaaaataattcaggccaggcccagtggctcacgcctgtaatcccagaactgtgggaggccgaggtgggtggatcacctgaggtcaggaatttgagaccagccggatcaacatggtgaaaccccatctctactaaaaatacaaaaattagccgggcgtggtggtgggcgcctgtaatcccagctactttgggggccgaggcaggagaatcacttgaacctgggagggagaggttgcagtgagccaagatcacgccactgcactccagcctgggcaacagagtgaatctgtttcaaaaaaaatagaagacttcaaagtagggactcaaacaaacatttgcacacccgtgttcataccagcattattcacaatagccaaaaggtggaagcaactcaagcgtgcgctaatggacgaatgcataaacaagatgtggtctatccatacaatcagccttaaaaagaaaggtgattctggccgggtgtggtggctcatgcctgtaatcccagcacttagggaggccgaggcaggcggatcatgaggtcaggagatagagaccatcccggctaacacggtgaaaccccgtctctatgaaaaatacaaaaaaattagccgggcgtggtggcaggcgcctgtagtcccagctactcgggaggctgaggcaggagaatggcatgaacccgggaggtggagcttgcagtgggccacgattgcgccactgcactacaacctgggcgacagagcgagactccgtctcaaaaaaaaaaaaaaagaaaggtgattctgacacacgctgcaacatgcatgaaccttgaggacatgacgctaagtaaaataaaccagtcacgactccacttctgtgaggtccctagagtagtcaaattcatagggacagacagtcgaatgccaggtgtcagaggctggggatgggagaaatggaagttttttaatgggtagtacagagtttcagttatgcaagataaagagctctggagattggttacacaacaatgtgaatgcacgtgacagaactataacttaaaaatggttaagatggtaaattttatggaaattttacaatgattttttttttttttttgagatggagtcttgctctgtcacccaggctggagtgtagtgacatgatcttggctcactgcaacctccgcctgccaagttcaagcgatcacctgcctcagactccgcagtagcatggaaggcacactcccccaacaccgtaccagtaaaataatctcctctctcctcccagcgcatattctcatacataccagccaccagattctgatacttggaatccatattaacccccgccccctccgcgaacgatcgctctccctacccttccgcacaccaccaccggtgaccatccctctacacccccgttacccaaaactctcatcattcacggcttctgcccagtacgatgcatacctcactccctacccaacacgagcccttcagcctccgagcatcgcctacatcggcacttccatgcattgtggaccaatgctctctaattccctccaccaacaccgaacattctcacctctcctgtataacccttccttccgctatccccatcataaaccccgcgttgccctctgaacggcctctcactttaacgagaactcttgctctccccatcgtcctatctcgcc

SEQ ID NO: 2 - DNA, 20 bp, Artificial Sequence
  antisense oligonucleotide
ggtgctcgtcctcccgacct

SEQ ID NO: 3 - DNA, 20 bp, Artificial Sequence
  antisense oligonucleotide
tgccacctacctgagggagc

SEQ ID NO: 4 - DNA, 20 bp, Artificial Sequence
  antisense oligonucleotide
attcttaaacctggtaagaa

SEQ ID NO: 5 - DNA, 20 bp, Artificial Sequence
  antisense oligonucleotide
gttcacataccactgttctt

SEQ ID NO: 6 - DNA, 20 bp, Artificial Sequence
  antisense oligonucleotide
gcacattgacctacaaacaa

SEQ ID NO: 7 - DNA, 20 bp, Artificial Sequence
  antisense oligonucleotide
gagctcttaccctttgtgtt

SEQ ID NO: 8 - DNA, 20 bp, Artificial Sequence
  antisense oligonucleotide
tgcaacttacaaagttgtgt

SEQ ID NO: 9 - DNA, 20 bp, Artificial Sequence
  antisense oligonucleotide
tcttccgagcctacaacaag

SEQ ID NO: 10 - DNA, 20 bp, Artificial Sequence
  antisense oligonucleotide
aatgccttacaagagttgtc

SEQ ID NO: 11 - DNA, 20 bp, Artificial Sequence
  antisense oligonucleotide
gtgctgagaactaggaggag

SEQ ID NO: 12 - DNA, 20 bp, Artificial Sequence
  antisense oligonucleotide
gccctattacctcaatcatc

SEQ ID NO: 13 - DNA, 20 bp, Artificial Sequence
  antisense oligonucleotide
gaattgcatcctgaaacaga

SEQ ID NO: 14 - DNA, 20 bp, Artificial Sequence
  antisense oligonucleotide
ggaaaagtacctgattcgct

SEQ ID NO: 15 - DNA, 20 bp, Artificial Sequence
  antisense oligonucleotide
gaaggtgaggcttaatagac

SEQ ID NO: 16 - DNA, 20 bp, Artificial Sequence
  antisense oligonucleotide
cacgaggcctctgaaacaag

SEQ ID NO: 17 - DNA, 20 bp, Artificial Sequence
  antisense oligonucleotide
ccaagcttaccgtgccattt

SEQ ID NO: 18 - DNA, 20 bp, Artificial Sequence
  antisense oligonucleotide
gcaacatctcctgcaaaatt

SEQ ID NO: 19 - DNA, 20 bp, Artificial Sequence
  antisense oligonucleotide
ttctactcaccgcagaacag

SEQ ID NO: 20 - DNA, 20 bp, Artificial Sequence
  antisense oligonucleotide
atgcaaatagctgtgaaggg

SEQ ID NO: 21 - DNA, 20 bp, Artificial Sequence
  antisense oligonucleotide
caaaggatactgttggattt

SEQ ID NO: 22 - DNA, 20 bp, Artificial Sequence
  antisense oligonucleotide
agaaatatatctcaatgctt

SEQ ID NO: 23 - DNA, 20 bp, Artificial Sequence
  antisense oligonucleotide
agattctcaccatccagagg

SEQ ID NO: 24 - DNA, 20 bp, Artificial Sequence
  antisense oligonucleotide
acagacttacctgatctcgg

SEQ ID NO: 25 - DNA, 20 bp, Artificial Sequence
  antisense oligonucleotide
tgaagatgatctaagggaaa

SEQ ID NO: 26 - DNA, 20 bp, Artificial Sequence
  antisense oligonucleotide
tcccgcctgtgacatgcatt

SEQ ID NO: 27 - DNA, 36000 bp, H. sapiens
  antisense oligonucleotide
ttatgagtcttgtcatttcagacagtcttccctggagcactacatgactagtccagttagtgattttagaaatgtttctctggaccttttaaaatgtatccaagtctaattcctcatttgtttctagtttatttgtcctcggttttacagcttacatagtttggagtttcctgtttagttttgtttatttttaactcctttaggcctaagactcttcattgtttctctttcatctgagtcattataatagtctctcaaaagttcatattccactcttgctccccctttctcctgcagtacaatctacatgtcgcagccaaggatcaggtcaaggcattctcatgctctctccagtggctcaccttcttacctagtttgtgatctggccccaggacatgcagactgcctagtaggcaattaatatctgctgaattaattctttgtattgtaagtcatatcaggatttcttgggggttagcattatcttaaaaccacaaaaaacaataactttagacctaattggtttcatgacttattgaggaggcgaggaataggttaaagctgctttgcatacattttggaatagtctcttttgtctagtaaggatggataagtttgttaataaccagtattcacatgggtagaaaaaaagtgtcttgatttttaatcctacaagtagtaaaggaatggtagtcagaagttgaatcgtacttttaatgcctcaggcaggatagaatagtattgttttgttttgtatcccaatatgcctaactacttctctctctctcctttctgttttgcttctcatcctttcacccattacagtctcagtatgtgtgttacaccaggaggttgccttggcaggtcagaggatctgtcacagtgaaggcactgtggtaccctttgttttttaggcaaggatgacacatgctagtcaacttatttccatttgttgttccctccacttggcactaaatgagacttaaccatttttgaatcaaagtttataaatttcttacaaaaattaaggtttttattctctataataagcacatgagaaagactggttttaaattttaaactgtggagtgacgtaaaaacatgtttaattttaattatttgctttcttttgttcgtcttgtaagttattaatgtattaatactggtaacttctagattggaggaatgatttatcctaatgtttcttttttaaaaaaaactatcagtcattcatatggcatatgctaatcaaggctctgcggtattttttttcttatgttttttaatgaagcagctcttttcatgatctagcagtctgtgttactatcagtacgtaaacagtaaggactcaaattttaagattaaaacaagttcattttgttaacatcatgttttgttgcatcttgcagcttcttggtgaatttttggatgaagccattaaattaattgcttgccatcatgagcagaagcaagcgtgacaacaatttttatagtgtagagattggagattctacattcacagtcctgaaacgatatcagaatttaaaacctataggctcaggagctcaaggaatagtatggtaagtgtttacttccaaaaattaggcaaagaatcattaactgctaccttttctcctctcgtaatttagataccttggcaaatatttaacttgctttgaaaaattaaattaaaactaaaaattaaacgaaaactattccacagtaaattgtttgcttcaggatcaatggtcttttctttattattattattaaaggttgagtatcccttatctgaaatgcttaggaccagaagtgtcagattttggaatatttgcattggttgagcattccaaattcaaatcagaaatacttcagtgaacatttcctttgagcatcatgtcagtgcccccaaaatttagattctcgagcattttagatttcatattttcagatttgggataataacctgtattattcataagatacaactctataaactagcactgccatctttaagtataaactatgattaaatacttgggcatagccattgaagacaaatatctctatcagcaaagaataagtggcatgcttgttaatttcatgtatacatgtattcccatagataattgtttaataagaagcttcacttagtcatcactttcacactataatgaagcgtggaatttcagaaatcttattctatactggggaatgaagtcagctaatatctcactgtgtgtgtttcaaaattcatccatatgtttaaaattgatttattagctaatttctacaaaagcctcaggctatatcagcataattctttataaactgaaatgcagatcactctttgggatagttctgatactgattgcagatcactaaattagcataataataaactagtgttttggggaattataagcctcactcaagaataattatatttttattacctcttcttgaaacaaatttcagaatctattctaaacaaaatgtaaactgttaagtatttataatccagtatttctcctaggttctaaaaaaactgttttacatgtgcacagggagtcatgcataggattggtgtttgcagcaccatttataacagcaaaaattggaaatagcatcaaatctatcaacagaagaataaaaaattgtgatacagtcatgctgtgaaatactatataacattttaaacgaactggagccatatgtgtcaacaaggataacctcagaaatgtagtgctgggcttcaaagcaagatgtagaatatatatatacatcattataccatttacatgtagcttaaaatatgcaaagcagtaccatatatttatggattatatatatgtacaaaagattaaaacatgcataaataccaaacttaggatgttgtttatctctgagaaaatagagaaaagaaaggaggtttgacctataatattatatttctttctaaaaaattcagagcaagccgggcgcggtggctcacgcctataatccccgcactttgggaggccgaggtgggtggatcatgaggtcaggagttcaagaccagcctggccaacatggtgaaaccctgtctgtactcaaaatacaaaaattagctgggcatggtggcacatgcctgtaatcccagctacttgggaggcggaggcaggagaatagcttgaacccgggaggtggaggttgtggtgagctgagatggtgtcactgcactccagcctgggtgatagagtgagactccgtctaaaaaaaaaaaaaaaatcagagcaattatggtaacgtattaagatttaataaatttgcataatagaatggcatttattatctttcctgcattcttgaagtaatttacaatatcaaaaattatagaagttggagtcattttgtaagccgttttgactggcctgttctcattgtaaccaaccacatttaaccaaccataaattaaatttaaagaaaatcatattatatagtcagtattaggattctgaaagccagctctctgttcagattttgttttttgttttttgtttttttgtttttttgtttttttttgagacagagtctcactctgtcgcccaggctagagtgcagtggcgtgatcttggctcactgcaagctccgcctcccgggttcacgccattctcccgcctcagcctccggagtagctggggctacaggtgcctgccaccacgcccggctaatttgtgtgtgtgtgtgtgtgtgtgtgtgtgtgtgtgtgtgtgtatttttagtagagacagggtttcaccatgttagccgggatggtctcgatctcctgatctcgtgatctacccgcctcggcctcccaaagtgctgggattacaggcgtgagccactgcgcctggcccttctgtatttattttattgtgtccaaaaggatagaagtaatgtgtttacatagaattattcagcaaatcttttgagtagaatttatatgctttttttttttttgagggagtctaccaggctggagtgcggtggtgcgatcttggctcactgcaacttccgcctcccaggttcaagtgattcccttgccttagcctctcaagtagctgggactacaggtgtgcaccatcacacccagctaatttttttgtattttagtagaggcggagtttcactgtgttggccaggatggtctcgatctcctgacctcatgatccacccaccttgacctcccaaagtgctgggattacaggcgtgagccactgcacccagcctcgtatgcattctttatcctaccttttcttgatggaagtacaaggttaaggatttatttaggctttaggctatctagtggagctaaataattatggatttgtacattgagtgcccacgatgtactaggtggatctttgaggcaacacaacgaacatgcagggtctctgttggcatagtccactgtctatttggatccattagcattttaggaaatagttgcaaaaaaatatatgttgtaatagatatagaagacacatgttgagcgtcatagacttggaagggatccagttacttgtctttggagaaagtgagaaatgtatatgactgtttcatgaattcagtttacagatttttgcttgaagtttttttgtgtgtttttgaatttcttattacagcgcagcttatgatgccattcttgaaagaaatgttgcaatcaagaagctaagccgaccatttcagaatcagactcatgccaagcgggcctacagagagctagttcttatgaaatgtgttaatcacaaaaatgtaagtgaacatttttggtttcctaagtatagatgaaatcaagatttattcatgaatatgtgaatatcaaagactaaatattaggggctttaaattgttctgtattaaaacattgtttaaaagggatatatatatatatatatatatctacagggtgattttcctcaactttattaaattgtatcagaagaatggcttctaaaatttagattatatgatttcctgtcatttaatttacaaaaggagttttaaaaagataagcgtttgagaagaattttattcagcttgatctcatcttctgctttttgttctccgtcaggctgacatttagaaaactgtagcattagcacagagatgaaattgttcccattatgctttgacactctgactttaatcatattgaatataaaatttatatcacttctccctaccccactcccacctctttaagtgatggcagtattcactgatcactttatgatgaggatagtgggtttagcagaaagtgtttgaaactatctagagggtattaacatgtttctaattctattttctaatgattgcaaagataagtctttataaagatatataagttgcactcattttgagaataaacagttatacttttttaaacttacattaagcttattcatacttttgtgattgttctaatggaatgatttcttcagttgaggaaataaactaggagtgaattgtgtaagggacacttttaggcagtcctaaaccgaaggcatctgactaaagacatttcgtagtgtttgtccaaaagagtttatataatataatgagcacagtaccttctgactacagataatacaatttaccacattttgagatctaaatatgtgcaggcctctgtgtattggagttactgggttcattatcaagggaaaaaaaaatcatggaagttcaggattaggccctttatttggggagcttacaatggtgttttgaatacaaaacagacatacagagagttaaatatcaaacatcacaaagcagtggccgtttgtcaaatgatgatggtggcccatatattgaaatctaaaaagagggacgatttctgtgagctttaattaactgggaaggctttggggaggagtagacctaggctgactcttaaagatggggtaaaaattggattgttgaaggaaaggagccttcagagaggaagaatgacgtaaaaaaggtccagagacaaagattaaacttagataataatagggaggaagttgtggtagccattcttgttccaagcgggaacttgatgtgttgctcatcacagttttgctggtggcatcctgtccatggatagagctgctcgtgccccttgtggcctgtgaacgcagttccatggcagtggtggtgttgccttagtttttccttggggaggagttcagttaatcctacctctcaagcctgggtgactgcaaagaaaggtggttccttcaactgcatggagtatgactaagcatgtctcacatgattaggcctctagcaggggtggcagaggtttatccaggcagaactctcagcacctagaagagtgtggacaagagtaggtactcagaatatatttgttcagtgagtgagtataaagctaaatatgggaaaactggaaatggtaactgaattattccactcacccatggatggatgtttttgtagtgttttgccttttaaacaatttcatcctctgtacagattgtctcttcactggtctaagcatctctggtttttctcattcttttcatagatggtcttgagtttcttttttaatcttggccgtttatctctgaacacactttgaaagtgacctagcacacactactgcataagccacctagcacacagtactgcacgtctgatcaaatcctctgagaggagctgaggcatgtcatctcttaactgtagacattaaaaccctgtttagaaatttcgtccccatctgactcccgttgtctttctttgttacaggcctgccttacagttcccatctcacccagtctttatattttttcttttgattatggagaattaagtatagtacttcatgtttctccctattaaatttcaaaggtgaaatgtggttgctcttcctggtctactagtgattgctgtgattgcttttttcttccttgttctgacttttcacttgtgggcttcacttcccagttttgctcaggatatctttgtaatagctggattaattcattagcaacttttgagcatattgcagaaattacgtgtctaactatcacagttatccagctgtttccaattcatcatcttattcacatggatgttattattagaaacattttgtaaaatgccattttgaaatcttcatatactgtgtctggttgcatttttctgatttattgctattgttctttgttgtttttattcatggaaggaaatagtatttctcaatgaacttttacctgctcttggtaattgttacttcttttgaagtagttatataccactactaatcattgacttttgcttggctgtccagtctataactgatataatttcctttatttccctttttgaaaaatgggacaactatctgtttgaatctcttttataacagctttattgaaatataatttctgtgtcatgcaattcacccatttgaagtataccattcagtggtttttagtatattcagtgatatgtgggaccatcactacagtcagttctagaataattttatcacctcaggataaaagaaagaaatcttttatcctttagctgtcattcccctacctcccatccttctcagttctaaacaaccactaatcaatcttctttctgtctctatatgcctttaatatttaatacctctcttctactctagggcttcccagatattaatgagaatagatttagtatttgtccaagtctcctattgtgcattgcatgcggttggtagacttgaacttatttagagcagccagatgctttcttattatcatttccctctcttaaatttgtttggccctttctggtgtattatttttccgaatttattcactgaacaaatatttaaataccttatgtaaagctgaatgcttaaaagaaaaacatcaattctgcccttataaagtttatgcctaaaaaacaaattcccatccccaagtatcatcgtcagcaaacatttttatatgtgtgtcatgtaacttgccatatactaggcagtaaaagtgattaagacacagttgtttctaataaggattctctgtggtagggatgaggctgatgtgaaaatgagactagattacagtgtgctgaataccataacaacattagaagataactggggacaaagtagagagaatagtcaactctgtcaagaactaggagttgtcagaatctacaaaagaggttcacctggaagataattaagttgagtagataaatgacaagcatagggaatagtatgtttaaaagcatcaaaacattttaaaaatgacattcttaaaactagcatagctaaagtatgaagtgtaatataggacttagcaggagatgaagtgagttgatggcatatcatggaggcttttgaatatactgcaagggagtttcagaatatatttgttggctttaaatttttgaggaggtaagtaatatgatcaggtctttcttttaccatattataggcagtaaatggtagctggatttgtagaaaaccatcgttagtgacccttaatcagctcaggccaccataacaaaatactgtacactgggtgtcttaaacaacagagatttatttctcacaattctgtaggctgggaagttcaagatcaagttgctggtcaatttgattcttgatgagggcctgcttcctgacttggaggtggccaacttttcaccttgtcctcatttgcctttcttcagtgtgtgctcaaggagagggagagaaaaattgctcttttcgtcttcccttgttttaggccactaattccattatgagcgccccactctcatgacctaatctaacccttattaccttccaaaggccccatttccaaatactatcacattgggtgctaggagttaaacatatgaattgtgtggggtgtgtgtgtgagggacaatcaatctgtaacactagtgttagaaggaattacagcatgaacagtctagatgttagtcttcaagctgtttatagcacagggaatggaaaaaagagattgaaggtcagattgacagtagaatggacagcattctatgagtatttattatggtgcatatactgaaaaaggagttaaaaatcaccctgagccttcttttgtaaaagagagggcacactggaaaagtaaggggtttgggtaggaatgaaaatgaattcagtgttaccttattttgtgagatgtccatgcaaaaagtgaaaagaaatgggttaaacacaatagtgtgagaagggatatgcttatggatgaaccagtgaaatagactgggaatgaagaaaactgggcaagtgcagtattatgaaaaaccaagggaggaaagggctgagattatatagagatactgaattggaaaagaactggtagaagggatttaagatttgctgataaattatgtagaggtttgtataatgtaagtgcttgataattgactgttcataaatggaattacagataaatgggttacagatactctgtgcctccacctatccatcccctcctccaactcctgcccataaagttgcaatatataaaatgaaagcaattaacttgagcttagaatgtaaagaaagattttaaactgatggtagttttttttaactcatgtatttgtagttcccaaattaaaatattatgaagtaatttctaatttttctgtctctcgacttttattatagataattggccttttgaatgttttcacaccacagaaatccctagaagaatttcaagatgtgtaagtgtaataattaaaattttgttaagttagtacatttttcttagattgctgctggacactttagctgttctcttttttcactcataaagttacatagtcatggagctcatggatgcaaatctttgccaagtgattcagatggagctagatcatgaaagaatgtcctaccttctctatcagatgctgtgtggaatcaagcaccttcattctgctggaattattcatcgggttagtagaagaaactatcgtcatactctttgttttctcattgaggtgaaattcatgtaacaaaattaaccattctaaagtggcatttttagtacattcacagtgctgtacaactaccacccatattaaattctaaaatattttcattacccccaaaaaagtctccatgtttcttaagcagttgtgcatctttgccctggcatgataccagtctgctttctgtgtctatagatttgcctttatgtttccatttatatgaaatacggcgtgatgttttcaagatttctccatgtttaccgtgtatcaatagttcattcctttttatgactgaataatattctatataccatatttcgttcatccattcatccatcgatggacatttgggttatttctacctttcagctattatggatcgtgctatgaaaattcaagcacaaatatttgtttgaatatatattttcagtcctttaggatatacctgtgactggaagtgctgagtcatatggtgattctatagttaactttctgaggaaccatcaaactgcacagtgactgcactattttacattcccactagcagtgtatgcgggttccagtttcttcacatccttgtcagtacttgttatctgactttttaaattctagccatcctaatgggtacgaagtggtgtttcttgtgattttactttgcatctccctaacgactaataatgttgggcatttttcatgtgcttgtggccatttgtgtatcttctttggagaaatgtgtattcaagccctttgctcatttttaaattgggttgtttgtctttttgttgttgttttagcatttctttatgtatttcatacatgaaacctttatcaggtatataatttgtaaatattttctcccattctgtaggttgtctgttcactttgctgatgatttcctttaatgcacaaaaactataattttaatgaaatacaattgtttatttcatcacttgtgcttttggtatcatgtataagaatccattgtcaaacccatggtcatgaagatttagtcttatgggctcttctaagagttttatagtttaagtctcacatgtaggtctttgatctattttgagttaattttttgtctggtatgaggtaaggttccaacttcattcttttgtatgtggcaatttagttgtgccagcaccatttgttagagactactcttaacccattgaatggtcttggcacccctgtcaaaaaacagttggtcatagatacatggtttcatttttggactctcagttctgtttcacatagctcctatgtgcctgaccatgtattatactagtaccacattattttgatcacttttagcttcgtagtaacttgaaatggagaagtatcaattctccagctttattcttttacaatattgttttagcttttcagagccctttgtgatgccatatgaatttgagtatcattctatttctgcagaaagggcaattagaattctaattagtatttcattgcatctgtagattgcttaggtagttttgccatcgtaacaatatttagtcttccaattcacaaacataggatgtcttaccatttccttccttctcttcttttttcttttctttcttttctctccttccctcccttcccccctcccttccccttccttcctttctccaccctctcctctcttcccctcccctcccctcccctcccatttgtcttgtcttgtcttctccattctcagctcactgcaacctctacctcctgggttcaaacaattctcctgccgcagcctcccaagtagctgggattacaggggcctgcccccacgcctggttaatattttgtatttttagtagagacagagtttcaccatgttggtcaggctggtctcgaacccctgacctcaagtgatctgcccgccttggcctcccagagtgctgggattacaggcgtgaaccaccccacctggcctctttcagcactttagatgtatcatccattgctatttgttttgccatggcttccagtgataaatcagctgttaagctcattgaggatcttttataagtgacaaatgcacctctcttgctgctctcaagatgcctttttgtctttcgctttcaacagtttgattatgtgtcttagtgcagatctctttatgtttatccatcttagtattcattcagcagcttggatatgtagattcatgtcttttttgatcaggatggaaggaagctaaaaagaaaaaatagattcatatcttttatcaaatttagcaaattttcagtcattattttcttaaatattttttctgctcatttttctctttcttctccttttggaactcccattatgcaaagttgatatgcttaatggtatgccatgtcttgttttctcctccactttaatgttttgtcccaggcagcattgggctatttacttgccttaaccatgttttcaaggaatgcctctgtctaaccttggaccagggtcccacactgaaaatgtggctgctttcttcaaaatcctttgctagttagggaggcaggtagagccaaagactagttaaaatgctggaaatatttcccaatgttttttccccacctttttattgtagtaaatacatataaaatgtgccattttaaccattaaatctacagttctgtggcattaaatacattcataatgtgcaaccatcaccaccatccatctccagaattcttttcatcttgtgaagatgcaactctccacccaccagacagtaattccctattctcctctccccgcaacccccgacagacatcattctattttatgtctatatgattttggctactataaatacctcatataagcggaatcatgcagtatttgcctccttgcaactggcttatttcacttagcataatgtgctcaaggtttatccatgttgtagcatatgtcagaattttcttccttttcaaggctgaataatattccatttatgtatataccatacttgcttatccattcatcagtcagtggcaattgggttggttccacatttagctcttttgaaaaatgctgctatgaacatggttgtccaaatatctctttgagaccctacttttcagttcatttgggctacagccagaagtggaattgccggatcacatggtcattctttttaacattttgagaaactgccacactgttttccatagcagctgtaccattttacattcccaccagtagtgcagaagggtttcagttattcctcattcttaccaacacttttatttttttcatagtaaccatcctaaagggtgtgaggtggtttcctgtttttaagttgccattttcttggttctgagttcacttgattgccgtaaacctttgaataatttccagaattctgaaaaagtttattctgacactttgatgttttatggtgcttctgtggagctattttggctgacatcactgcctctcatcaaattcttagtatcactgtatttcactgatttatatggagcaatgtaaagtttgtttttgctctgaagtgaagcagtatacaaaataaactgctgttattaccagtcattccaaattgggaattgaatattaccaagttaccaaaattgagtatttgccagaattgttatatatatatatatatatatatatatatatatatatatatatattcagaaatattttatatttttgttctctgggatttttacctgcttttttgctatataatttacatgccatacaatttattcatttaacgtgtaccactcagtgtttattatatttgaagattgtatgctcgttaccacaatcttaactttagaaaattctattgccctaaaagaaactccacatccactcatctttatttcccattcttctgtgttcctttcacctctagccctaagcaaacactaatctactttctgtctttgtatatttgtctattctggacatttcatgtacatagaattatataatatgtgatattttgtgcttggcttattttacctagcataatcttttcaaggttcattcatgtatttattgtgtgtcagttcttcttgtcattttattgacaaatatttcactgtatagatatattgtttatccatttatcacttgattataatttgggttgttaccactgactattacaggtaatgctgacatgaacatttatgtaaaatttttttgtgtgttggtatgttttgtattttagttaaatctagcttattctttgaaaagtattgagaggatttttttttttttttttcctgagagacagaatcttcctctgtcactcaggccaggatgcagtggcgtaatcatagttcacttgcagccttgaactcctgggttcaataggttgaaaagaattttttttttttttttttttttttttgagacagggtctctgtctgtcacccaggcatgagtgcagtagcatgaacatggcttactgcagcctttacctcctgggctcagtgatcatcccacttcagcctcccaagtagctaagactatggacatgtaccaccatgcctggcaaatttttattttttatttttttgtagagacggggtcttgccatattgcccaggctggtcttgtattcctggcttcaagcaatccagctgccttgacctcccaaagtgctgggatcacaggcatgagccatcatgcccagcctggaatttttaaaaattatttaattcagtggaattacatcttccttctgagtcatctgcgtcaaataaccttactcatgtaataaaataaatacagagagagttttcacagtataagacaattatgtcttagggctattggggctttttttttaagtgaacaatcctgctacaggcttatccttaattatcattgtaagtgaattccttgtatattacatatacagctgctctaatcatttgtctaatgcaatctttaaataacataataaaaatctctttaaatgggagagtattttatcttcctgagcccctactcatacagtatgtgaagtagagcagacgagtgaaacttctaggtttgggttgtttgtttcctgagtccttagaccggggcagtaatccaacattgtggaagttcatttgattttagtaaagcacacccctccaaatagaaaggttccagattaatgaggatagcaggtggctggaccagaaaaacagaccaaatggtagaagatagaaaacaatggaaacaacaagaaccacaaaaaacaaacacaaaattacaaaagagcaatatttatggtaatatgttaatcggattagttccttaatttaaaaaggtcagattttggcagcacacaaggctcaacaacaactgcgtatttccataacacaaacattcgtagctaacttcaaaaggctgaaggcagagaggttggcagacatataccaggaaaatgcaaacattaaaacaagggtaactctatttaatgagataaagttgaatatggtggggtcagagaggaagtgttcctctctgagaataaagagtctcgcgttttcaaggtgaaagatactatccaattaggatacacaattatacatgaagtagggttgctatacaaaaagcttaaactgtagaaaatagaagaatggcacaagcacagttgtattgggatattctaacttactcccttagtatcagtacataaagtggtaaaaatgaagtacatctgtagaaaatttcttaatgtgataaagtggagctaatgagatctactggggtctgttatctattatgaagaatctccctttatcttggcttgggctctttgggaaactctagagtggggatacgcatttaagttttttgcaggtggtcttgaggccacgattggtactcatctctctgctctactacttattctagattcacctccacccttggctagtacttctgctagtctgaggttacttacttggtaaaataacctgacctttcatcccttgggggttcagggcaagtcaggattgctactgtgagaccaaagctgacgaggtcccaccatctagatagttgcgggtcactttgtcggaaagagaaagccaagtgatgtgcatggatggctcttctttgcttggaagtgacacacatgacttctgcttagagtgcattgacaaagctagccatagacttgcttaatttgtaaggaaactcggaaatgtggggagagcagacttcagcatagtggtgagccccagcctctgccacagtctgttcttctggctggcaaacattcctttgctccctttattccacaccttcttaagggaaataacccaaagttctattccatcaagacatttatcaaatccaggcattgttcctcttggtccagaaacataggaactggaatgaccaaattatatggccaaccctataaccactgctcccctgccaaataccatagatggagtggtcagacagggccagtgttatcacagtgaacaatcccatttagaaaactgaaaaatgcagtcattggttagttgtaattaaattcttctgtacagacattgtgagtgtcccttgccgtgggtggggaatattcctaatcagtcctcctgactgatctctgtggttctctatagtacctggctctaccctgtgggagagtcatctttttgctatcctcatgaatatatctaaagtggatattgaagaatgtattttccttgagggctgctcacctttctcagcctgcttcttacctataaaagattggggcccaagggtcattttaaggccatccttctcagccttgccactattgacattttggagcaaacagttctttcttttgatgatggaatgtttagtagcattcctggcctctacccactagatgccagtagcactccccagttgtgacaatcaaaaatgttttctgacattgccaaatattattttggggcaaaattactgtgaattgagaactcctgtttgaagtgtagaaggatcatagacctttggcgttaggtctgacagttaactaactctttcagaaatttaagagatttcttatctttctgattgtagttcctttcatagtatcttttttagctcatgggtcctgtattcttggacttaattgaggctgtcttcagtcttcctgtaagctttcaagcttctgttggcagactgaggcaaatttatcaattgaaaattttacaggactttttccctgaaagggtttcataattattatgtgaaaatgatgcatagaaaccaacccaagatatcagtggcttaaggcaatatttattgtttacttgggtctgcagggttggctgtgtgacactgctccaaactgtgggtcaagttcaggcttgtcccatattcaggtttcagcatctaaactgaagcatacaccagagacatccttttctcatggtgaaaggtagaagagcaagagctatgctgagtcaaacacattcccccatattccattggacaaaataagacaagtgactgaacctatcgatagggtgaggacacagtctggggtgagcaggaatatttgcagaacaatctaccatactttcttccaggggtctagtagcatttagcttttccaaaatggcaagaccctgaaattctaggctctgttcccttttatttctgctttaaaaggtggctacttcttttaaatgcaaccagtagctaccaacacaaactactgaagttttgctttccagtcttttctagagcagggttcaacaaactttttctgtcaaagccatgtagtaaatattttaggctttgtggataatagtctgcgtcacagctacaagactctgccattgtaacacaaaagcagccataggtgatctgtacttgaatgagcatgactgtgtttcagtatcagcactttctttacaaagcaggcaggccttaatttgccagcccctgtgctagagcttcaggttcattgtacaagtggctgtgcaggtgacagcttcatgaaatgttttgttactacgtaacctagatcacctttccagccttcgatgtgagtttccctgctgctcaactgctaagccaggatcttatgtttttataataattaacaccccacctctgacaccagtttcagtgttagaatagactcagctgtactgaataacagataaacccttaattctcagtggcttagcacgactcaggcttatttcctttgctgaatgtgtggtgcagatcatggtgggtttgtggactgtgcgtttacatagatgatcagcaacccaggctgccagagaatcctctggaaagttcctaatcaccaaagcagggcaggagatgacaggaagttgtgcactggcccttctcttctttacttccactcacatcccatgaactagaactacccacgtgcccttgcctaactgaggaagctagaaaaattgggggaagaagacagaatgttgggttagttccactgcctgcataaattctaagttgctattcttatatatagttagcagtgtaattaacagctgtaaggtacatattgatatatatggatgaatttcaattaatagtctttgaggctttttttgacaatttttttgcttaacaaaaatatctgtctttaaaaattgttactttgctcttaattttttcacaatgaagtgcttgaaattggaatgttatttctaaattggaatctgctagtataaagagccagtaaccttcaaccatcacactaaacattgaagtagtatcaaagcttgtaatatttattcttaaacatgcagtgtttctttgctgaggtactgtattttagaagtattagcaacattatatttacagaataccaataatgaaaatttttttcttctgtttcattaagacctttgaattgttggtatatttaatttcataaaaccactagattacatatcttctaaacccatccatgattgcctgtcgaaatatgttaaagacaacatacatgcttttctgtatgttaacattacaaaagtaatacaaaagtaatagttgtctttcaagcaggctattttccttttttgtattaatcggtgatatttaatatgtatactgcatgtcagtacaggtaatacagtgcatttgtgttgtggatttatgctttcttcgttttttggtcctttggcttataatagtattcatcaaagctaacaggcaaagtatgttaaacatgaagccatgagtgtttatgctgctgcctgacttttgatgggcacagcacagaatatgttaaagtaggggccaggtgaaaaattctgaattattcagtagtatctatagcaagtaattttatagattagtggcaccaaggcaatggtgtcctacttctacaaacagacagaaattttatagcaaatttgtgtgacttcaggtgggaaatcagggatggaaaaatggataatgactcttctagtcctttggaactgggtatctgccagtctaaaagatcaaataacatttgttaggattgacaagatggctcgttgttgacttaaaacatctgtataaagcttattgtgactttaaaatgagattttaaaaatattggtaatatgttatgcaaatgaatataaatgctaacttatgactttcttttactatagtagtttttttatgtgagctttgcgatgtcttttcaaatcccttttactctaaccaaaaatgtctagctaatcataatttctagcaattaaaacattactaacttctctaataactgtatgtattcataaattttaaatcccataaaactgacttctcaggtttgccttttaacgatgaatcagtgcagtaatattacagtgagaaaaacgaacaattaacatgaatgttttgcaagggatagtataactttattgtgaactgtaggattttcctatatatcatggcagtcattttttaatttttattttctgaaggacttaaagcccagtaatatagtagtaaaatctgattgcactttgaagattcttgacttcggtctggccaggactgcaggaacgagttttatgatgacgccttatgtagtgactcgctactacagagcacccgaggtcatccttggcatgggctacaaggaaaacggtcagcacacacatttatttgaaatatttttctgatttagcttttttctttattcagtagatttttaatgtaaatacttaagcagaagtacgttgagttaaatgtgtatcattgtttgaaatgtgtatcaaaagtttgcaggtaactataaattttttcatcaatgtttggaaaaacttggggcccttatttgtttaaatacggatacataacatcagtactaccattggatgaagaatttgttgccgtggccctgagacagaatttatgctctttgcttgcctataacttgaaaatgaacatttctaacatggtctcagggatgtatagtgtactgacagtttattattagtgtacatcagtgatatttgcctatatttccaaccccatgaaactaaagctcatagcagtagcactcattgtaaattttaagagatacattaaggcctctgcaaagctgatggataattaaggtgacctcttgaaggaagcagtaaagtttcattaaaacgttgtatccagccttaaggaaatgacttttagtattttgtatgggtatatccagttgtatcaatcaacctaagaatcagtgttgaacaaaattatctggcatgtagatcatgaatttaaacattttgtatgcatatacctacttatatttaatttaggaagaaattttgttgagtcttagaaatttaggagatgaataaatctgctaagaagcatgggattttatggaaataaaccatcgttttaaaaaatgtggtgacttcatccaaaatactaccaagaataaaacctagacattttttctggcaaggaatctggaatagcagttcattttgaatcaggaaattgtaataagtcaacttttaagattctttttttataaaagatacaattaagctgagcttgattttctgagctgattaaaaacaaatactttaaacatttgctattaattttttattcttaagtaaatatgctgttctttttacttacactctacctgaaatttgaagttttttattttttgggaattgataacttcacgtgacccaccagcatgaagattgcttttgcatttgctgtaattgattgtcatagatatggtgttatgtgtcagtgtcctataaatatgatcttaggatagctccctgccagaaattagcactaagaaaaactgtcatatatagaattgaggctgggtatggtcgcttacacctgtaatcccagcactttgggaggccgaggctcgtggatcactcgaggctaggagttctagaccagcctgagcaacatggcaaaaccctgtctctactgaaagtacagaaattagtcaggtgtggtggtgcacacctgtaatcccagctattctggaggtgaggcatgagaatcgcttgaacccgggaggtggaggttacagtgagccgagattgcaccactgcattccagcctgggcgacaaagcaagactctgtctcaaaaaataaaatagaataaaaaatgaactgacatatggttcctctttagttaagaattatgtagatcaccttcttgccttcccactcccaccctaggaaacggagtcaaatgtaatgcttaatgattataactacattagccagcaaggttaaacataagtacttcctccttttaatgacacctcacactcattttatttatcttatttatttatttatttatttatttatttatttatttatttttgagatggagtctcactctgttgcccaggctggagtacagtcatgccatcttggctcactgcagcctccacctcctggcttcaagccattctcctgcttcagcctacgagtagctgggactataggcatgtgccaccatgcctggctaatttttgtatttttagtagagacagggtctcattatgttggccaggctggtcttgaactcctgaccttaggtgatccacctgcctcagcctcccaaagtgctgggattacaggagtttgagccacaacgcctggcctcccacccattttaacaactttgctttatgtaccttttattgcaggctactcagagcattttggaaagcagtgtaatcaatatatttttaactgcttaatagactaagcagttagtttacagctagagttgtgcatgaatcagacttatgttcctagtcttcactggcccagataatatcttctctcatgcttgcagctttcaaaatacttttgtttaaaaatgagcttttctgaaagttaccaaataagttgtaagctggatatttaaagtttaacattctctataaaatctgtagagaagatttattctaatagatttttgtcaaaaacggaaaccttagaaaaaatttttctctgaggcactaatttataaatattaaattttttttctaaaatcttatccttgaaagctgaaccatgacataaatatgaaaatattcattcattttaatatgacattttagggaaactttagttccacagtatttccaaaagtaatttccagtttagctcttaagactcttcagtgaactggcagccttttaataagaatgtactgtattagaaagtacaggctttaattttctaagtcctttaacatgagtaaagggcacgtgtctttctgacatctatttgtgccgttttccacttgtcattttaaagaattgggaccttcagatgtcacaactaaatgcaagtttctaaggcttttccttctaaattgctcattcttccctcttttcctgttacactgcaagcacttactctctcctttttgtgtgatgttcaaacccaaaggcataaaatgccacgatgtgttgtcagtttcccttagtaagtggagattccaaattgtctgtttctgtgtctagcagattcttgtcaacattgattgggattcccatttacagttggcaagggtcatcagtaagtcttccaggagactcttcatactcaattcgtgggaactgacttactgtatctagccaaaactaacttcttaagccaaaagcagtttggttttatgggtttaaaaaactttaggtaggaaacatgaaatacatttctcttactaagttttccttgattagttgttcattctctgtgcacttgttccttttgggatagtagggtaaatgtagcatgatgttgccaagaaaacaaatggacttttaattcagaacaagcatagcacttctcagttaaatgtatttgggaaatccagtttaaaaattccaaagagctttagattaacttttttatcataattattcatagagattatttctgttagatattttaatatgctgaattattttctcatgcttttttcttagagagtataatgcacagttgaacttctaaatagtatagtataacaattttatttttataattttactacctcatgtacatttttatttacaaagtttgtgtttggttctatttcatattttataaacaagttctcagggacaaacatgcagggtttttctatttatgcaatttctgtgcatagctctcactttgaaattagatatgtagttaaaatgccacactttacattttcttttgtgaaccagactttcaaaaaataatttgtgttttaaattattccgttagctaaagttacaatcactttttttcttttcgtgacgttttgcagtttttatataatgcagtcatattatgcttctttgattttaatgaccttttgctttgcttttccttcttttgtgctgcaaacatatagtggatttatggtctgtggggtgcattatgggagaaatggtttgccacaaaatcctctttccaggaagggactgtatccttgtgctgctgcagcagttaattagttaggcgatgaacttctttatgttctctaatgaaaatgaatatgctacatttacacagatgggtttttaaacaggcataaagtttgtggctattatagttcaaaaattgttgagataagaagctgaaatatttgtaggctgcatctggcagtaggacatacagtctctgctggtagtcagagcacattcactgtcactcatttttattttatactgctttttataattttaaagtatacatggtgtgtgtgattttatttctttctttttttttttattttaaccaaaatctttatgttaatgtcattgcattttgttttcagttgacatttggtcagttgggtgcatcatgggagaaatgatcaaaggtggtgttttgttcccaggtacagatcgtatccttatctttggcctaaaatgtagtttctaaaggtcaaaatgtatgatagcacttcagtttggtcaggtataatgtattttgtctctccctaatatattgttaaattactcttaaaataccacctactatttgacatagacttttccttccgttatttattgtttcatatgttaattctgaaccctgctcaattgtaattatgcacaattacttgctggctttgagttgatttaacctaaatcaaaaatcatggcttgcattaaaaatttataataattatacacttaagtctagaaatacgtacaacttaatatgaattttggagctgtctgttgcttctggccatccttttgttttgtcccctacctcctttttgttaatgttctgtgtggtttgtgtcttttgattgttgtctcattactcacataacatactgaccctttcatctgagaatttcagcagtagtagttttgtatggtaacgatagctttggattcatactttttgttcatcccactacttttaatatttatattaacagtgatttgttatgtgtttaccagctagtaacacttagaattgtttcaggaggaagaatgggaaaaggcatattcacaaggttacaataagtgagttttaggccttggccttcagggtcctggagtgtaaagactagaggaaaggagaccaagaaatcctaaccttacaacttagcaaaaaactgaatcttttttataagggtcagagctttagaaaatttttttaaacctaagctaaacaaagaatggcaaaagaaaatgccacttttatcgaagcctttatttttattacaactgattattttctgttagtgtagaaaactcagtaaaacaggatgtgttctagaaatattgtctaggagatataatcatgttttctgataaattgacagaaaaggaaaaattttgtatattattatagcattgtgtgcagtttgcagttagagccactatggatagtaattgtcattattcttagttgcttatcattttatttagaatttgtgagtttaagcttccattttaaggtaaaatcagttagtttgaacacacaataaataaggttaataaagcttatttattgatcatttctgtccacctacaatcattgccttttgcagggtgcctgtgagatataaaatttataactgccacatcctttcttaggaatttttaaatttctattttcttgtaatatgaatatgactaatgtattgaacattagttatggagtatttttcttagctacttgatattagatattgatcagtggaataaagttattgaacagcttggaacaccatgtcctgaattcatgaagaaactgcaaccaacagtaaggacttacgttgaaaacagacctaaatatgctggatatagctttgagaaactcttccctgatgtccttttcccagctgactcagaacacaacaaacttaaaggtactttttacaaatatgtacatttaatcccatttggggtgtgtagtgtgtgtgtatgggtttgtgtgtttatatgtattcatattcttatgggacatgaacccaaggttttctctggatggtggggaaaaaaatgaggtttttgttttttttttctttaatcttatatattttaatcatatgtataagataattttacagtaatatttttaaaacatatgctttttaaaaaatctcaaattgctgaaagttattaataatttgagaatctttacaaatatatgtacattaacaccattatgtttgcagccagtcaggcaagggatttgttatccaaaatgctggtaatagatgcatctaaaaggatctctgtagatgaagctctccaacacccgtacatcaatgtctggtatgatccttctgaagcagaagctgtaagttattttcttaatgtttacagaacatattgcattcttagagttagaatgacagttaggtttggaggagaccttttaattttaaataaaaatgtagatacatgatgatgatgtttttctgtttcttcatgaagactacgtcaaataaactaatgaacatattcgagcccctcctacacaaaataaagttacctcccactgttttttgcaatcttgcctggatacctaaccagagaactaggatgttgaatgctctgggggaacatcctaactcaggtataaaacaaattactgtatccaaaggaaaacagaattctgtgatctgtgatataaataaaatgtggcaatttcaagagctagaagaaaagaaaaaagacagttaaacattttatttcctgcaatgaaggagtcttcctaaactattatgtctgtataagtaagttgatgattgatcagtcttgatctaatgatatatttttataagtcatctgtgtggctaatatttcaaataactacagagttaaaatactcccagcatactgacttggttattattgccttgtgtttttcagccaccaccaaagatccctgacaagcagttagatgaaagggaacacacaatagaagagtggaaaggtacattcgtcagattcttagagggaaaactgtgaaggagcttctggtttttatatggtgatttattatcatgttagagaaatttgtgactttaatatgcataaccgaaatgtggtaatattaatatttttacataagtagaaagtaagtctgcttccttccttaacttaatcttaagttcccaagtttcccaccccagacacagacacattagtgctctgtctcatatttttttccatggtttgtgaatacacaaatgtgtttagtgtctcccacctctcctcttccacccttttaaaatcacgtatggttgtgtacggtgtatgctatatgtacttgctttgtttatttattatatatttttttgagttggagtctcgctctgtcacccaggctggagtgcagtggtgtgatctcggctcactgcaagctctgcctcccgggttcacgccattctcctgcctcagcctcctgagtagctgggactacaggcgcctgccatcacacccggctaatttttttgtatttttagtagagacagggtttcaccatgttagccaggatggtctcgatctcctgacctcgtgatccacccgcctcggcctcccaaagtgctgggattataggcgtgagtgctttgttgatataatcagatatctggaaggttattctcttttagttcacgtatctgcctctcttgctttaataactggttagtgttctgctgtatgaatatgtcacactttatatatccatctccctcttggtgaccatttaaattgtgtccagtctgttgctgttagaaacaactctgcaatattcatgttcacatatgagaacatgcctggaagaaaaatcctggaaggagatgttctgagccaaagatatttttcagttatttccaaattactctccaaagaaatggtagtagtgtacacttccgtccatgttatataggagtctctctttgccctcacccttgcctacagaatgggttaatttttaatatttgtaaatctaatgtagttttattctattttatgaatgtggctgaaattaatcatgctttgatagtcatggaagttttcaaatatttcttcaaaaggtgccctgggacatgtgggtcaggtaatttttaggtcccgtttacttccatttgtgttattgtccttcagtgttttgaccacttttgtaactgcgtgactttgagtaaatcactcccccttcatctcaccctcctacactgccccctgcattttacctcatcttaaaatagaggcagaagacctgttattaagagttgtctctaaatcctgggaaaggaaaggggactggggaggtataaacatgaataagtgacccatctataaatgtattttgctaagcatgaatttgattctttcttagaaattgaagagatttagagattggttttctctgaactttgggaaacccatggttagcagagcccgtgataaagttagaagaatgacaaaatgataaaattggatagagtctgctgcatttgaatatgtaatgtgcatttgaatatgtataaagatatagctctgtactgagtatgtatgaaaacattaacctaatatttttacatcctactaatttacagtagacgatgaagtattttgtagaatcttgtggtttttttggttgttttttttgtttgtttgtttttttgagacagggtctcactgtgtcccccaggctagagtgcagtggtatgatcatggctcactgcagcatcaaaactcctgagctcaagtgatcctgctgcctcagcctcccccgtagctgggatcacaggtgtgatccactgcacttggctaattttgttttttttttttgagacagggtctcactgtgttgcccaggctggtttcaaacttctgggcttaagtgatcctcctgcctcagcctcccaaagtgctgggattataagtcttaagccactgcgcccagcctaatttttaaaaaaaaattttgtagagatggggtctcactgtgttgtccaggtcatagactcttaataggccagcagttgtaaggcacaccattatgtgccacaaagaaaaaaacaccttcggttgtacagcaccattggttataagatatagtcaatttcagagattaaactgtgaaaaaagtacatcttaaaatcagtgagatacagtgttttcatttgtataaggatatatttgggggttttgattgctttaaaaacatttacctttattctgtatcctttactcctagccccaggtgcatgtcagtaattacccacagactgcctttttcaagatctacttaagagttttagcgcatagcagaaagaaagattaattgccaaagccattaattacagatggctttgcctagttagtgctcctaattagttttggttcttctgccttaatccctttgtgctttttcccagaggagtgctattttctctcttaaaaaatcctcttactgcaaatgtttatcattcctttttgtttctttgaagaaaaccatctcttattctctcctttatcagtgctttctatttttctccttccaagccttaagttactatagcaaacaaacttatctccagttgttgttcattccaacatattcatttgttttaccttattaccaatataaatgtcatactctgtaatcagggattctttatcagaattttattcttcaggaattaacacagactccttgaaattcgatgcaatattttttgtttgtacatttttctcagaagagggttcactacttttatacaattctcaaaagattccatgacccaaagaagatgataaatagtgttgatgtggcatccaccattaaggttaagtgtggtgtgccctgtgagtctgaatgtctacttaagaaccttaagtagacattaagaaccttaagaaggttttttgtttgtttttgtttttttgttgttgagatggagccttgctccgttgcccaggctggagagcagtggcgcaatctcagctcactgcaacctctgcctcccaggttcaagcaattctcctgtctcagcctcccgagtagctgggactgcaggcgcctgcccccaagcccggctaatttttgtgtttttagtagaaatggggtttcaccttgttggtcaggcttgtctcaaattcctgacctcaggtgatccacccacctcggcctcccaaagtgctgggattacaggcatgagccaccacacatggccgaaggttcttcttaagtagacattcagactcacaaggcacatcgcagttaacatcagaatcacttctgatgataatataagtgaagaatataagacaggaggcgcatatattaataccagcagagcagctcccagtgtgtctcttcagttggaacagttgttgcagtggtctacttgctgtccagaagcctgataagagaaaaagattcccatggagaaatgttcttcgaagtgataaccatgcttactcataaggagttgaaatgtagcttacctgctagttttcctccaataaaaatgtgtttatctttcattctgatttgttgtgaagcttttgcacactctaatttaaatcttggtagcatatatctagttgagtacccacagtgcaccaggctctattccagggcccaggaaatggaagtcagtaagacacgtggttcaagccctccctgagacagatggtagtacagaatggtatgtggtatgatgtgctgtagcacaagttgctggtagagtgaaaaagaaagcttctaccccagcactatcaaaccaacttcagaaacagtgaattacaggaaagattagtacttccttttaatatgatccattgttgagtgtcaaggaattgttattaattaacatccttgaatcttaggccgacatttaactgactgtcattgtaaggacactgtttgaagtacttcacatgtataaaaatttccacttaaaccatacatgcgttgtgagattggctcttagactttgaaaagttcatttttgtttacttcttttacagaattgatatataaggaagttatggacttggaggagagaaccaagaatggagttatacgggggcagccctctcctttaggttggttacaatataagcttggttaagattacagtttacttcttgtgttgtaatcttcagtggcctgaaacctgcagttcttcccatatttacaaaatcattattattccaggcttaataagtataaggaaatacagttttgttttttctactaatacattatactaatatatcagtaactgttcataagatgcacatctttttctatgatactgacattctgaagaacagaaatttaaaaactttttgttggcattgttgctgggtctttaaagaggaaacttctcaaaattcaatatacatacctttctatgatcttgacagtctttactttggataaataaaagcttcactgcaaaatttagtacatgtaataccaaattgctgtctttctctttttgatattattgatttgttgaatgaaggcaataacattaaaaccatcactagaaagtattctttctctaagaagaaaactgggtttgtaggagttaaaacttttttttatcataagctgatcttatatttaatgttagtacaagtaaaagtataaagaatagaggggaaaagttaaatggcaggtaacatgtacactaagtacataccacataacagacactagtggtttatatactttatttcattcagcccctaagatcctaaggtataggattattgcacacatgttatggatgaggaaactaaagttcagaaatttgaagtaacataagattccaaatctattatgtagatgaactaatacagtaacttcagagtgtgtggttttttcagcctcccattttgttatctggctggcaacagagacttctctggctacagaggttaggacagttgtatgaaggaggtgaaatttgagctgagccttaaagagtgagtagtatttcaaaagatcttgattttgaagtaaaactatgccattttaattcctcagaaacttctacttttgaggaaaaaaatagatgttgtatctagcatcttgtatatgggtaaggttttttaaactatagcgacattgtatactataaacataattgtttaagccattttttgtggcttgctttgacatttttggttatatattttagagttgtatattttaaatctttgatcaagaatgcaatcttccagattatagtgtagatcctgttgaatatatgaattggttttgaccgcttttacctattttggaaatggccttttctctacaatttacttataacaaatttaaagctctattataaatgctttgtgtaattaattagctttgtattgctatatagtagtagtagtaacaattgttcatgatggaggctcaggtgggatttgaaaagttcattatgtgggacagttttatactttagcatactatccaagtgagtggcacagctggagtgccagatgtttgagtaaatgtaataatttcatgagttagagcatttgtatttgttcttaatttgtaagtgaataatttgaatcttagtccagcacttgcttatgatcacaaaataagtcagtgaaaaagatagaaattgaggtttctagactttttctggatcctcagttatagcttgcaaagacgagtattagcaaattaagctgttataaaaatattctgctcttgattttgtactaaaacagaaggagtagtgtttggtaaatcaaaataccagataaccacagtaccatttccacttgatttttaaaaggaattttattctttttccctgtcgagtgccttcctatctttgttttggtttggctaatagtaaagtaagtttacctgccttgagtgtatagaggctcacttaagagaggaatgacccatgtgagactaaagattttccatattattaccattcagatatttgagaatttactgtattgctttaaagagaaaacaagtgtgtgttttttccccttaggtacttgatttttagattaaaaagttaacaatgcatttaaaagtcaatttttatcagattaagacatttgggtaaaataatagaccctgaactagaggcatatataaaaattgtatatgttggagcccttttatggtttgaatgtttcagtacaagtcttagaaactagtcattgtgtactatgtatggtacacagatataccatactgttcagtcagaaaaggctcattccaagtattgattgaactaaatagaatatactatctgaatttcactctgactgggaagctaatggacctttcttgggtctaggagattatcacctcttttacctctcatctctcaggcctgaaatgctcatcctgcttttctcttccgtttcagctcccatcacatgctttgtctcttgtggttccattcctttctgtatcccagtcccctccctaaagattttcctattcccactacactgcctattttctttttgcatttgaagaaaagctcacagaagacttttcatattgaagtgtttcattgctcatctggacagaatggagggatgatctcaaatacagatgctgggttcaggagcagtggttttcagcccatttggtttcagcatctttgggtgcctgaacctcaccctcagaaattctgctcagtgttccatttgaagaccattgattttatttcataaatgtattcttgagaacttttaagtaacttgcattattccaatttggaatgacccttatttagtgttcatgtggttcagaagtacttagcctagtgcatgagttacctttaactatccttcatcccccagcataagtcattcctgtccccttcctaaaccaatccccttgagaggatttctgtctcgagctcaggtattcctgttaactttttaaaatccaggaaatgcttgttaggtaatactttcggcaaaggaaactgtttgctcttactatatttaataaatccatatttctgcttatcaagtattagagtagaaataagaagacccaagtttacttaactaggccacttgagtgacagtggcatgtcccataacctcgtgtaaagtggggcagttgaattgaggtttcttcctgttaaacttaattttattccttgtcttggcatttgctttaaaacaagatgtgccagaagtacatcttgtttcaaatttgaatcatttgaatttttcctttttagtgagaagctgtaaagacttttttgtagggaagtagcttttaacttttgtagttacacagtcctttaagatcctctgtccaaaaaaaggcattacagacagttttgcatgtattatcagcagtattcacacataccctgaagcccattcatggatcttgctgcaggaccatttctaaatgtggttcagatgtaaaattcttgtcttaaactgaaaaacacattcattgaaaggataggactccacgattctagacattttcagaattctcacctcatagctgtcaatgaagagtgtttttaagttagtgtgttggatatcatttgcgattatttttagtgagccttcgaaacccaagagaaaaaaattaccactggaggcagtcagtgcagtgcaagtagcttgatctgcagctgtctgcaactgatttgctgttttgtttctcatagcacaggtgcagcagtgatcaatggctctcagcatccatcatcatcgtcgtctgtcaatgatgtgtcttcaatgtcaacagatccgactttggcctctgatacagacagcagtctagaagcagcagctgggcctctgggctgctgtagatgactacttgggccatcggggggtgggagggatggggagtcggttagtcattgatagaactactttgaaaacaattcagtggtcttatttttgggtgatttttcaaaaaatgtagaattcattttgtagtaaagtagtttattttttttaatttcaagtgatgtaatttaaaacctaagttgtgtttcaaaacagcaacaaaactgtattgtattttttttgctgtaattaactgtataatgtaaacctaattattttatcatggtttaaattttttgcatatttgctttatcttatgctgctgatttttttaactgaatttgtaagattttgtttatcaaagcaactattatgtggtgacttgcctatatcatgaattatttaagatttttatagttttttttaattagaatttatttcagatgttttgttcatgatactatccttcagggttatgtgcttatcaatgaaataaccccagaggagtgagggaaaataacttgtagccagttatattcaggaataactactgtaaatgatgaacgtgttaggagacctccaatatttgctacttgccaatcctaatttagttacaagaattggtaggcaatcctacttaattttggcaaaagccccgtcatctaaatggcagaataactcagagcatgtctttgaagatgctgggcgtctaccaccaccttatgtccccaccctacccaacaaaaataagtaaaaagaatatggtgtattctacaaatttgtggcatgctcaaagtttatgatcacataaaggcaagaggatacttcatgaataatacatttcaatgcaaataaacagatggttcacttctactagctatgagcctgtttttgtatacactgagttaatctactcaggctgtaggtcccagcaatgttctagagtctggtctttccctttcctgcagcttcgggtccttggacctttcctgtttcctattacttggagtgtctgtcagttgagcaccagttgttctggtgtttcatttgattctacttgtagcataatcatttatacgagctattgggaggttccaaaccctacctagatttgtgtaggtgatgtatcaaatgagcaatataccgttcatctgaaaatagtagcacacagccatatataggatatcattttctaag

SEQ ID NO: 28 - DNA, 20 bp, Artificial Sequence
  antisense oligonucleotide
ataagctgcgctgtaataag

SEQ ID NO: 29 - DNA, 20 bp, Artificial Sequence
  antisense oligonucleotide
ggccaattatctataataaa

SEQ ID NO: 30 - DNA, 20 bp, Artificial Sequence
  antisense oligonucleotide
ttacacttacacatcttgaa

SEQ ID NO: 31 - DNA, 20 bp, Artificial Sequence
  antisense oligonucleotide
gactatgtaactttatgagt

SEQ ID NO: 32 - DNA, 20 bp, Artificial Sequence
  antisense oligonucleotide
ttctactaacccgatgaata

SEQ ID NO: 33 - DNA, 20 bp, Artificial Sequence
  antisense oligonucleotide
gctttaagtccttcagaaaa

SEQ ID NO: 34 - DNA, 20 bp, Artificial Sequence
  antisense oligonucleotide
gtgtgctgaccgttttcctt

SEQ ID NO: 35 - DNA, 20 bp, Artificial Sequence
  antisense oligonucleotide
cataaatccactatatgttt

SEQ ID NO: 36 - DNA, 20 bp, Artificial Sequence
  antisense oligonucleotide
acaaggatacagtcccttcc

SEQ ID NO: 37 - DNA, 20 bp, Artificial Sequence
  antisense oligonucleotide
tgatcaatatctaatatcaa

SEQ ID NO: 38 - DNA, 20 bp, Artificial Sequence
  antisense oligonucleotide
taaaaagtacctttaagttt

SEQ ID NO: 39 - DNA, 20 bp, Artificial Sequence
  antisense oligonucleotide
gcctgactggctgcaaacat

SEQ ID NO: 40 - DNA, 20 bp, Artificial Sequence
  antisense oligonucleotide
aataacttacagcttctgct

SEQ ID NO: 41 - DNA, 20 bp, Artificial Sequence
  antisense oligonucleotide
ttggtggtggctgaaaaaca

SEQ ID NO: 42 - DNA, 20 bp, Artificial Sequence
  antisense oligonucleotide
acgaatgtacctttccactc

SEQ ID NO: 43 - DNA, 20 bp, Artificial Sequence
  antisense oligonucleotide
tatatcaattctgtaaaaga

SEQ ID NO: 44 - DNA, 20 bp, Artificial Sequence
  antisense oligonucleotide
tgtaaccaacctaaaggaga

SEQ ID NO: 45 - DNA, 20 bp, Artificial Sequence
  antisense oligonucleotide
tgcacctgtgctatgagaaa

SEQ ID NO: 46 - DNA, 20 bp, Artificial Sequence
  antisense oligonucleotide
ctctctgtaggcccgcttgg

SEQ ID NO: 47 - DNA, 20 bp, Artificial Sequence
  antisense oligonucleotide
ctttccgttggacccctggg

SEQ ID NO: 48 - DNA, 2947 bp, H. sapiens
  CDS (178)...(1896): antisense oligonucleotide
gaggattgcatctgtctcttatagttttgaaatctcctaatagcaagaccagctaagggattgtacctttttcctacaaatataaatatatatatattttaaaccaagtctttttttccggctctctttgctttaaagctgtcctcttgaaattacttccccccgccccccggagagatgtcttatcaggggaagaaaaatattccacgcatcacgagcgatcgtcttctgatcaaaggtggcaagattgtgaatgatgaccagtccttctatgcagacatatacatggaagatgggttgatcaagcaaataggagaaaacctgattgtgccaggaggggtgaagaccatcgaagcccactccagaatggtgatccctggaggaattgacgtgcacactcgcttccagatgccagaccagggaatgacatcagctgatgacttcttccagggaaccaaggcagccctggccggaggaaccaccatgatcatcgaccatgttgttcctgagcccgggacaagcctattggcagcctttgatcagtggagggagtgggcggacagcaagtcctgctgtgactattcgctgcacgtggacatcacggagtggcacaagggcatccaggaggagatggaagctctggtgaaggaccacggggtaaactccttcctcgtgtacatggctttcaaagatcggttccagctgacggattcccagatctatgaagtactgagcgtgatccgggatattggtgccatagctcaagtccatgcagagaatggtgacatcattgcagaggaacagcagaggatcctggatctgggcatcacaggccccgagggacacgtgctgagccggccagaggaggtcgaggctgaagctgtgaaccggtccatcaccattgccaatcagaccaactgcccgctgtatgtcaccaaggtgatgagcaagagtgctgctgaagtcatcgcccaggcacggaagaagggaactgtggtgtatggtgagcccatcactgccagcctggggactgatggctctcattattggagcaagaactgggccaaggccgctgcctttgtcacctctccacccttgagccccgacccaaccactccagactttctcaactcgttgctgtcctgtggagacctccaggtcactggcagtgcccactgtaccttcaacactgcccagaaggctgtggggaaggataacttcaccttgattccagagggcaccaatggcactgaggagcggatgtctgtcatttgggataaagctgtggtcactgggaagatggacgagaaccagtttgtggctgtgactagcaccaacgcagccaaagtcttcaatctttacccacggaaaggtcgtatctccgtgggatctgacgcagacctggtgatctgggaccctgacagtgtgaagaccatctctgccaagacgcacaacagtgctcttgagtacaacatctttgaaggcatggagtgtcggggctccccactggtggtcatcagccagggcaagattgtcctggaggacggcacgttgcatgtcacggaaggctcaggacgctacattccccggaagcccttccctgactttgtgtacaaacgcatcaaggcaaggagcaggctggctgagctgaggggggtccctcgtggcctgtatgatggacccgtatgcgaggtgtctgtgacgcccaagacggtcactccggcctcatcagctaagacatcccctgccaagcagcaggcgccacctgttcggaacctgcaccagtctggtttcagcttgtctggtgctcagattgacgacaacattccccgccgcaccacccagcgcattgtggcgccccctggtggccgtgccaacatcaccagcctgggctaaagctcctaggcctgcaggccacgtggggatgggggatgggacacctgaggacattctgagacttccttccttccaatttttttttcctttttttgagagagcctgtgatagttgctgtgggcagccagttcctggggcttcctcttgggccccctgcactcggtctcccctggagtttctgaattcgctcacccaagtccctacacagtcatgaacaccacacccaagcccagccacccaccccacactgagctgcatccaacatgcagacatgcgccaccatgcagatcccagcaagggtgcccttatcacatccttggctgtgcagtcagcaccttcctgtcacggggaagatttagtgaattaccctgagctgccttcttttcttttgaaaaatttttaaaaatggttttctttgtgggactggggagggatgggggggtgggagttttttttttttaatactaaattgaaagtctgattcaatattaatccttgggtcttgaactggacatcctaatgatcaattacttaaccattaagctgattccgaggctggcaggctaccgccgcccctctggaaaggttccatgtgtctgtatcacccatcccttactcttctggtcagctgttgagaagagactggttttttctttggcctagattttgcaacagattagaccttttgaaggttctctaccatttttctgtgtctccggtctgttctggctttttcttctgcactcttggagagatttagatgttggtctcctggtttgtgtttcttcgagacaatgtgcttttttccctggctttttgtttgttctcaaagccaggcatctgaatttggcctcagacacagcctgagcggaccctagttttgacccccactccatagttttgtgctagcctggtgtctgtttaagattggtgctagctgattcccgtcactaggaggtggctgagcttgaggcttgccagacacagggatggtcctgattaagtcaccaatatgtcacatgtgggcccagataggtcacttgtggtggaa

SEQ ID NO: 49 - DNA, 218 bp, H. sapiens
gaggattgcatctgtctcttatagttttgaaatctcctaatagcaagaccagctaagggattgtacctttttcctacaaatataaatatatatatattttaaaccaagtctttttttccggctctctttgctttaaagctgtcctcttgaaattacttccccccgccccccggagagatgtcttatcaggggaagaaaaatattccacgcatcacggt

SEQ ID NO: 50 - DNA, 93 bp, H. sapiens
agagcgatcgtcttctgatcaaaggtggcaagattgtgaatgatgaccagtccttctatgcagacatatacatggaagatgggttgatcaagt

SEQ ID NO: 51 - DNA, 189 bp, H. sapiens
aggcaaataggagaaaacctgattgtgccaggaggggtgaagaccatcgaagcccactccagaatggtgatccctggaggaattgacgtgcacactcgcttccagatgccagaccagggaatgacatcagctgatgacttcttccagggaaccaaggcagccctggccggaggaaccaccatgatcagt

SEQ ID NO: 52 - DNA, 169 bp, H. sapiens
agtcgaccatgttgttcctgagcccgggacaagcctattggcagcctttgatcagtggagggagtgggcggacagcaagtcctgctgtgactattcgctgcacgtggacatcacggagtggcacaagggcatccaggaggagatggaagctctggtgaaggaccacggt

SEQ ID NO: 53 - DNA, 66 bp, H. sapiens
aggggtaaactccttcctcgtgtacatggctttcaaagatcggttccagctgacggattcccaggt

SEQ ID NO: 54 - DNA, 85 bp, H. sapiens
agatctatgaagtactgagcgtgatccgggatattggtgccatagctcaagtccatgcagagaatggtgacatcattgcagaggt

SEQ ID NO: 55 - DNA, 73 bp, H. sapiens
aggaacagcagaggatcctggatctgggcatcacaggccccgagggacacgtgctgagccggccagaggaggt

SEQ ID NO: 56 - DNA, 125 bp, H. sapiens
aggtcgaggctgaagctgtgaaccggtccatcaccattgccaatcagaccaactgcccgctgtatgtcaccaaggtgatgagcaagagtgctgctgaagtcatcgcccaggcacggaagaagggt

SEQ ID NO: 57 - DNA, 161 bp, H. sapiens
aggaactgtggtgtatggtgagcccatcactgccagcctggggactgatggctctcattattggagcaagaactgggccaaggccgctgcctttgtcacctctccacccttgagccccgacccaaccactccagactttctcaactcgttgctgtcctggt

SEQ ID NO: 58 - DNA, 146 bp, H. sapiens
agtggagacctccaggtcactggcagtgcccactgtaccttcaacactgcccagaaggctgtggggaaggataacttcaccttgattccagagggcaccaatggcactgaggagcggatgtctgtcatttgggataaagctgtggt

SEQ ID NO: 59 - DNA, 175 bp, H. sapiens
aggtcactgggaagatggacgagaaccagtttgtggctgtgactagcaccaacgcagccaaagtcttcaatctttacccacggaaaggtcgtatctccgtgggatctgacgcagacctggtgatctgggaccctgacagtgtgaagaccatctctgccaagacgcacaacagtgt

SEQ ID NO: 60 - DNA, 184 bp, H. sapiens
aggctcttgagtacaacatctttgaaggcatggagtgtcggggctccccactggtggtcatcagccagggcaagattgtcctggaggacggcacgttgcatgtcacggaaggctcaggacgctacattccccggaagcccttccctgactttgtgtacaaacgcatcaaggcaaggagcagggt

SEQ ID NO: 61 - DNA, 170 bp, H. sapiens
agctggctgagctgaggggggtccctcgtggcctgtatgatggacccgtatgcgaggtgtctgtgacgcccaagacggtcactccggcctcatcagctaagacatcccctgccaagcagcaggcgccacctgttcggaacctgcaccagtctggtttcagcttgtctggt

SEQ ID NO: 62 - DNA, 1024 bp, H. sapiens
aggtgctcagattgacgacaacattccccgccgcaccacccagcgcattgtggcgccccctggtggccgtgccaacatcaccagcctgggctaaagctcctaggcctgcaggccacgtggggatgggggatgggacacctgaggacattctgagacttccttccttccaatttttttttcctttttttgagagagcctgtgatagttgctgtgggcagccagttcctggggcttcctcttgggccccctgcactcggtctcccctggagtttctgaattcgctcacccaagtccctacacagtcatgaacaccacacccaagcccagccacccaccccacactgagctgcatccaacatgcagacatgcgccaccatgcagatcccagcaagggtgcccttatcacatccttggctgtgcagtcagcaccttcctgtcacggggaagatttagtgaattaccctgagctgccttcttttcttttgaaaaatttttaaaaatggttttctttgtgggactggggagggatgggggggtgggagttttttttttttaatactaaattgaaagtctgattcaatattaatccttgggtcttgaactggacatcctaatgatcaattacttaaccattaagctgattccgaggctggcaggctaccgccgcccctctggaaaggttccatgtgtctgtatcacccatcccttactcttctggtcagctgttgagaagagactggttttttctttggcctagattttgcaacagattagaccttttgaaggttctctaccatttttctgtgtctccggtctgttctggctttttcttctgcactcttggagagatttagatgttggtctcctggtttgtgtttcttcgagacaatgtgcttttttccctggctttttgtttgttctcaaagccaggcatctgaatttggcctcagacacagcctgagcggaccctagttttgacccccactccatagttttgtgctagcctggtgtctgtttaagattg

SEQ ID NO: 63 - DNA, 20 bp, Artificial Sequence
  antisense oligonucleotide
aagagacagatgcaatcctc

SEQ ID NO: 64 - DNA, 20 bp, Artificial Sequence
  antisense oligonucleotide
ctggtcttgctattaggaga

SEQ ID NO: 65 - DNA, 20 bp, Artificial Sequence
  antisense oligonucleotide
atcccttagctggtcttgct

SEQ ID NO: 66 - DNA, 20 bp, Artificial Sequence
  antisense oligonucleotide
tatttgtaggaaaaaggtac

SEQ ID NO: 67 - DNA, 20 bp, Artificial Sequence
  antisense oligonucleotide
cttggtttaaaatatatata

SEQ ID NO: 68 - DNA, 20 bp, Artificial Sequence
  antisense oligonucleotide
ttaaagcaaagagagccgga

SEQ ID NO: 69 - DNA, 20 bp, Artificial Sequence
  antisense oligonucleotide
ggaagtaatttcaagaggac

SEQ ID NO: 70 - DNA, 20 bp, Artificial Sequence
  antisense oligonucleotide
ctgataagacatctctccgg

SEQ ID NO: 71 - DNA, 20 bp, Artificial Sequence
  antisense oligonucleotide
ttggtgacttaatcaggacc

SEQ ID NO: 72 - DNA, 20 bp, Artificial Sequence
  antisense oligonucleotide
accgtgatgcgtggaatatt

SEQ ID NO: 73 - DNA, 20 bp, Artificial Sequence
  antisense oligonucleotide
gatcagaagacgatcgctct

SEQ ID NO: 74 - DNA, 20 bp, Artificial Sequence
  antisense oligonucleotide
acttgatcaacccatcttcc

SEQ ID NO: 75 - DNA, 20 bp, Artificial Sequence
  antisense oligonucleotide
aggttttctcctatttgcct

SEQ ID NO: 76 - DNA, 20 bp, Artificial Sequence
  antisense oligonucleotide
actgatcatggtggttcctc

SEQ ID NO: 77 - DNA, 20 bp, Artificial Sequence
  antisense oligonucleotide
caggaacaacatggtcgact

SEQ ID NO: 78 - DNA, 20 bp, Artificial Sequence
  antisense oligonucleotide
accgtggtccttcaccagag

SEQ ID NO: 79 - DNA, 20 bp, Artificial Sequence
  antisense oligonucleotide
cgaggaaggagtttacccct

SEQ ID NO: 80 - DNA, 20 bp, Artificial Sequence
  antisense oligonucleotide
acctgggaatccgtcagctg

SEQ ID NO: 81 - DNA, 20 bp, Artificial Sequence
  antisense oligonucleotide
gctcagtacttcatagatct

SEQ ID NO: 82 - DNA, 20 bp, Artificial Sequence
  antisense oligonucleotide
acctctgcaatgatgtcacc

SEQ ID NO: 83 - DNA, 20 bp, Artificial Sequence
  antisense oligonucleotide
caggatcctctgctgttcct

SEQ ID NO: 84 - DNA, 20 bp, Artificial Sequence
  antisense oligonucleotide
acctcctctggccggctcag

SEQ ID NO: 85 - DNA, 20 bp, Artificial Sequence
  antisense oligonucleotide
cacagcttcagcctcgacct

SEQ ID NO: 86 - DNA, 20 bp, Artificial Sequence
  antisense oligonucleotide
acccttcttccgtgcctggg

SEQ ID NO: 87 - DNA, 20 bp, Artificial Sequence
  antisense oligonucleotide
caccatacaccacagttcct

SEQ ID NO: 88 - DNA, 20 bp, Artificial Sequence
  antisense oligonucleotide
accaggacagcaacgagttg

SEQ ID NO: 89 - DNA, 20 bp, Artificial Sequence
  antisense oligonucleotide
gtgacctggaggtctccact

SEQ ID NO: 90 - DNA, 20 bp, Artificial Sequence
  antisense oligonucleotide
accacagctttatcccaaat

SEQ ID NO: 91 - DNA, 20 bp, Artificial Sequence
  antisense oligonucleotide
gtccatcttcccagtgacct

SEQ ID NO: 92 - DNA, 20 bp, Artificial Sequence
  antisense oligonucleotide
acactgttgtgcgtcttggc

SEQ ID NO: 93 - DNA, 20 bp, Artificial Sequence
  antisense oligonucleotide
gatgttgtactcaagagcct

SEQ ID NO: 94 - DNA, 20 bp, Artificial Sequence
  antisense oligonucleotide
accctgctccttgccttgat

SEQ ID NO: 95 - DNA, 20 bp, Artificial Sequence
  antisense oligonucleotide
ccccctcagctcagccagct

SEQ ID NO: 96 - DNA, 20 bp, Artificial Sequence
  antisense oligonucleotide
accagacaagctgaaaccag

SEQ ID NO: 97 - DNA, 20 bp, Artificial Sequence
  antisense oligonucleotide
tgtcgtcaatctgagcacct

SEQ ID NO: 98 - DNA, 32767 bp, M. musculus
  misc_feature (1)...(32767): n = A,T,C or G
gaatgccatgaaaagccatcgctaattaaatttccccatgttaacctgctcaggtttatttaaaagctggggttttgcgccccccccccccccttttaattaaattggtatttggagctggctggtggtggcgcactcctttaatcccagcactctggaggcagaggcaggtggatttctgagttcgaggccagcctggtctacagagtgagttccaggacagccagggctatacagagaaaccctgtctcgaaaaacaaaaacaaaaacaaaaacaaaaacaaaacaacaaccaaaaaaaccccaaccaaacaaaaattggtatttggaaacgtcccacactcactcgtaaggatctgtcattgactcttgtgtaataggggcacgttataccactggtcctagtttctttgcttgtacaatgcagttgatggatgaggggatccttgcaatttctttttcatcttccatctttatatcacagagctgtctgtcaccatgtaggatggaaagagtctgtggtgctgtaaaaatacaattatttctagagggaaagaaaaatttttgaacagaacattgttaagtaatacaaagagtgaaaaacctagttgaagcagttgatagagaagagatgatatttggagtaagacagagcttgtacagcatccctgtagcatacagcatccctgtagcacacatccctgtagcacacagcatccctgtagcacacagcatccctgtagcacacaagcatccctgtagcgcacatccctgtagcacacagcatccctgtagcacacagcatccctgtagcacacagcatccctgtagcacacaagcatccctgtagcacacatccctgtagcacacagcatccctgtagcacacagcatctttcccaaatatcatttatggtagttactgcacagacctttctcaagtgctagagatttcttagtgatctcatttatttaaaatgaaattagaggggctggagagatagctcagcagttaagagcactgactgctcttccaaaggtcctgagtttaaatcccagcaaccacatgatggttcacaaccatcagtatagctaccgtgtactcgtcatatacatggtggctcacaaccattcgtaaagagatctgacaccctcttctggtgtgtgtctgaagacagctacaaggtacttagatataataataaataaatctttaaaaaaatgaaattagagtcaatcttcctccctggttaattaatattcttttaataataaacataattctattaggatatatatgcatatatgtatacatatatgtatatacatatatgtgtgtagttatacgtatatatacgtatatatgtgtacatatacacacacacatatatacacacacatatatattatttattttgcggtagctattcctggttttcaactcaactatatctggaatgaactacaatcaagaattggagggcatgccaggcagtggtggcccacgcctttaatcccagcactcgggaggcagaggcaggtggattttgggttcgaggccagcctggtctacagagtgagttccaggacagccagggctacacagagaaaccctgtcccgaaaaaccaaaccaaaccaaaccaaaccaaaccaaaccaaaccaaaccaaaccaaaaaccaaaaaaaaaaatataaataaataaataaaataagaaaattggagagcacacctatgatccagatcttgaggctgggagacacaagtttctgacccatatcttgacatggagatcttgaggtatagtggtcattaaaagcttagacccagttcttccagaggtcctgagttcaattcccagcaaccacatggtggctcacaaccctctgtaatggaatctgatgccctcttctggtctgaagagagcaatggtgtactcatatacctaaaataaataaatctaaaaaaaaaaaaaaaagcttagttccaggccaggtagcaaatgcctttaactccaggagactgaggcaaggagatctctgggttcaaggtcagcctgggacaagttaagtggggaaaagaattgctccgccatacccttccctgagaactgtgggaaaacaatgatggtcgcttcaagtcactgggttttactgtgatttgttgtgaagcaataggtaattgagttagttatcaaagcaccagcacggctaacttgtcttggtgtgctgtgaagaggttgcgacagcccaaggattcaagtttccaaagctgactcatcattatgaatgtggacccagttggccagtcattgctctatgcataaggcttaatggaaggagcgcctaacagtcacaagcgtttgttgaatgaatgaatgaatcattataagaatggcaataaggaccccatcagcaggaggttaatttaagcagatttggtctgttttcttttccaaaaaggtactccttcctccaaagtagttgataatctagaagtactgtccgtcttgatttcatatcaatgacactcctaaacctagacacacattatttttcttttattagaagtattaaaacagcaaactatgttcttttgggctagaactggagttaagactgaagtagaagatccagcagtcaaagtgaacagaactaaaattgcttcgtgtgtttccccgggtgacaatttctgcgtaccacagacagcagaggtctggtccgctccaggacaccccgttcactctcgacttgtctgaggctgatgatttcatacagaatagctcttgagacaggaaaggtgacagagtgacatttgcaaaagctttgggagaggtttaggctatgagaagcttatagacaggcttttttttttttaaaaaagatttatttatatttattatatgtaagtacactgtagctatcttcagacagctccagaagagggcgttagatctagttacagatggttgtgagccaccatgtggttgctgggatttgaactcaggacccttcggtagagcaatcagtgctcttaactgctgagccatctcaccagcccctagacaggcatttgaaattttctcttggaatacaggaattgcaggactcacagaggtgccttctggagaaagcatgagcaagtggtgtttgaaagagcaatagtggggtggtaagatggctccatgggtaaaggtgtgtgccatcaaacctgacaacctgtattggatccccagaagctacggggtgaaagcagaggacagactcttgcaagttgccccctgctgtgcacaaatgtgccaggacctgtgcacatgcgccctcatgcgcttgcataaacaacagatagatgtaaattaaaaataaaaagtagaaggagaaatcacagaataaataagaagaaagctttcagagctgtagaagaacagggcagtttaaattcaaagccaaatgtgctagctaatccattatatatctagaaaatgcttttttttgataaaaatagagaagcttgcttaaaaattaaagcgctagactgagctctcttgttgaggggtcagagatgtggggaaggagaaaggcactacagcagcagcgtgctcacacacgagatgcttttgcacagagcctacaacaacacatcaactatttatattacctacatccccttcttggtgtcccacttatcaagggaaagaattttctttggacatctttagcatgtagtaacaatagagttctggaatcagctgttggaaactatcattaaagctggtttatcatactccctaattattctatatagtcagtctccctagagctttacataattctcccttcgttataagcttctgtttctcccttgagtgttttaaatccagacaggtgaggaaatgaagcacttggaaaccagtgtattgtaatatctgtacagccaagcatataaatatatacattttcatatatatatatatatatatatatatatatatatatatatatatataaaattgctatatggactttttcccttttgccctactcacatcctttgagtggaataaagatgtgaaaaactccaaaaaattttaaaaggctgcacagaaacatatttgctgcgaaaggaagtaggggtgtatttagaaactcccaacaaggcctgatgctgtcagcgtggccagctgatgtcagaggggcccacacacctgccaacaccgcgtgtctgttccttcagccctgcagagcagccgagcgaagcagctagctggctttcggctttcttgcctcacttggtggtgcttgtgggctggggtcagcgctgggatgcgcctccttgtgctctatggagtgatgctctggaaacagaaacgggtccttttttttcctccagccaggcatcaggaagcttagggatgaatgtctttctttttctttagaggaagatttccgagcttcttagacgctccgagtaatgtcacgcaagatgcctccagggaagaggcaaaagagggtgatgctagtaacagggactctggggacaggaacgaatgtgggccatttccccttttcaacccacttctcttgatgtaacttcatccttatttttccccacagcagtagtagacactctgctgagcaccacttaggtttttgctgtgcatctcagctaacactttgacattggggattctgtgtaaactagatctccatcttagattaggctgtgtgaaccgaatttatttacatcgttagaaaccaaatagaggcctctcggctattgttctcagattgctctcattggtcatccctgccctccctctactgaccagaaccttgccccaaaacagcctgtaataaacatcaacgctggcttagcttgggctgctatctctggcggagagatctttaaaggatgtatctaaatgcaatgtttgagtagcttcagagagctctaatagaactgtaaatatccccggtttaattagcagtcctgcagttcggtaatggcccatagctctctgagccgagcctcttgaggtttctagacttcagaggctgcctgcaactatgctgtgtggacctatgaaattttccttctcctgtactctaaacccccagctagcctttcctagacacctactcgcaattattgcaaatccataactgactactatcctccggatttctaaaatgatccagtgtttcagcttaggtctcaactcagagatactttagggctcagattggcatcctgagaattaagtcccctgggaaaagaacaataaggaagaaaactctacctacattggagttgatgtcattttttttttccctccaagctcaaggtgatcgcttgctttgtggctggttggtgggggaggaggggctgtacgctagttatcagcatttctgaaccagctctctcaaccgcgacaggtcagccaatcccggcagtaagcttttacttgacaggtttgttctgggctgacagccattgactaggtgctcagataagtcacttggctgagtctacggtaggtggggcgcgctcaccagttcaggggcagtgactggaagtttgttgcaacatcggtaagcctaaccagccagcagcaacaggagatacccttttgccccgcgagtacagatctagaaagggttcacctcattaagcgaaggagatgcgtcaatcccccccacccccgccccgcgcctccccctagggcccggcctcttctcccacggttgggaacgcgcggtgtgggcagatccagaacaggagtctcgtgtcccggccttctggctagctctatgggttacaagcgaaagggaggaacagcttggggactctccgcgtcagcgtgcacaaaccggcggcggccagcagagaggggtggcgggggcacgtgcttggatgtggctgcttgtgtaaccagctccccaggcgctcggccccgacagcgctcctgcggacggctcgtggatgctattctctgctccgatccggcaagagaggggtccagcagaccacacgggagaaggaggcgggggcgatcacctaatagagcagaggggaccaagctcctgccccaggagcacacagataggggaatgggaatttggaaagttccccaactaggaccacacgtgacctcctcctgaaagtagttccgaccgcggctcatgtatccttccacctcgcctttgagccctcccaggcctgctcgccccgcccactcgctggctgcagcttccgaacgtcccatactccacacccgggctcagtaaccgggtcctcgaacatgcaaggtccgacagggtcagaacctggccatcgcgatccaattctgccgggttttcatagcggccacgaagtggggattgggggtgggggcttagctctttgaagactgagcttggctgtgatccggtagacccaccgctgcggggagctgcgggtctcatcaccgggcggtggaggggtgtgtgtgaggtgcactctattcacggagacccactttgtccaaccaggggtgtcctttgggccctggaaactcaggggagatgtgaatgtacacgccccgtatgcacaatcatcatgcttggctgggagcgttcatctttcgggcaaatgaacccagctgcctgggaagcaagaggcggggcagggaaccggagcccgatgaggtgacccacgcgggagacacaataggggttgttctttgtgcaaagactgacaccttgaggacaccgtgagggggagaggtgtgttatctaggtaaagactgtcgccgacaaatcctagcgaagcactgcaatctgaccacagcgcagggcagggaatgaaagccgttccgaagaaacgcagggacagacgcaggaaggataatcctgcccctgaggctcccggagcaccgaccaaggcggtcagctagtgcgatccacctgtgagcggtcagcgattgtgctcagcgcaccctcactcggccccagcctgttgtacctttgccgggtctctctgcgctgaggccaaagccggcgtagctccgggagcgagccgcggacacactgggcatgctccgcggcgttccccgcccctgtcccttccgacgccccgccccgccccgccccgtccccggctcagcgcccgcctcccgcccgcctcccgcctcccctccggctttccgaggcgccctgctctcccggcggggcggcggagggggcgggctggccggcgcacggtgatgtggcgggactctttgtgcactgcggcaggatacgcgcttgggcgtcgggacgcggctgcgctcagctctctcctctcggaagctgcagccatgatggaagtttgagagttgagccgctgtgaggccaggcccggcgcaggcgagggagatgagagacggcggcggccacggcccagagcccctctcagcgcctgtgagcagccgcgggggcagcgccctcggggagccggccgggcggcggcggcggcagcggcggcgggcctcgcctcctcgtcgtctgttctaaccgggcagcttctgagcagcttcggagagagacggtggaagaagccgtgggctcgagcgggagccggcgcaggctcggcggctgcacctcccgctcctggagcgggggggagaagcggcggcggcggccgcggctccggggagggggtcggagtcgcctgtcaccattgccagggctgggaacgccggagagttgctctctccccttctcctgcctcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaaggattcagtggataataatgtcctgggaaatcacagggacttccagatgtaaggcagagtgctctaccattgagctatgaaaccattcctttctttctttacttttttttttaaagagatttatttattttatgtatatcagtataccattgctctcttcagacacaccagaagaggtcatcagatcacattacagatggttgtgagccaccatgtggttgctgggaattgaactcaggacctctggaagaacaagcagccagcgctcttaaccgctgagccatctctccagccctctttctttacttttgagtcaagttttccttcactgacccaggctagtcttaaaccctggaggcccagaacttgtgatcctccagtctcaccctaccaaatagctaagcattatatagccctgcaccaccatgccaggttgattctgtttcaaagggtgttactggcacttgggtgtggtgcctgtaatcccagtatttagggaagacaggaggaacaagaggagttaaactttcctgctggcaagttgcagaccagttcaggctaagacacccctcttcccacaaaaagaaagtttgtcactggaaattaagttagttaatgtatatgcttacattctcatgtatgttgttattgcatagccattgtcagtgtttgatacggttttcttttcacaaagagttttttttttttttttggtttttcgagacagggtttctctgtgtctggcctagtatttgtttttgtttgtttgtttttttttttttttactttatttattatatgtaactacactgtagctgtcttcagacactccagaagagggagtcggatctctttacggatggttgtgagccaccatgtagttgctgggatttgaactcggaactttgaacctttggaagagcagttgggtgctcttacccactgagccatttcaccagcccttatttatttatttatttatttatttatttatttattttttgagacagggtttctctgtgtagccctggctgtcctggaactcactcggtagaccaggctggcctcgaactcagaaatccgcctgcctctgcctcccaagtgctgggattaaaggcgtgcgccaacacacccagcttgccctttctttcttaagcattttctttgtaatatgttacatgcatgttagggctttcagtgtcccttgttgaaagcactccagtaatggtaaatgtaggttgttcttgatgtctgctgacttgacaggccatgacgaggcttttccccttcaggctttcccttgttcttgactatgaccccatttatgcatatatgcctgagtaaattgaactacttgacaggcatccctaaacctgtgtctgttttatgtaaatcctgtcctttctgtgtgtctttatgagttgcattgggctcttgttcctggatagatttctgtctctttcctgcagttctctgcttggactgttctagccacttaagtatatcttttctaatataaatcttattttttatgtgtatgagtgttatgcctgcaaacatgtctctttcccgtatgcgtgtctggtcttcactttgatatgggtacagggaaccaaaccggatgctctttctgcaagagcagcaagtatgtttaactgctgggtcatctctccaaacactcctgtgttttcttctgtcaccagaaggcgtgtgtgagtgctacccaacataatactcacttggtgatgcttatacatacttccacggatccctctgaaaacatcttcatttaaaaaatacagtagtacttttagtgccatggtaggtctgtgtgcctgtctttcttgaggacggtaaccactgcccggccctacagactttttaatttgtctcatttattcttgcataatattatttagcctgtccctctatattattcctataagttaacattttttttctcaaaggctttgagagttggtgttaaagattcttggccattacagatgatgctctgcctttgtagtacctatggccaaagccttctcatgacttggagatcaattactgagttatatgtagaaggcaaatgtatccagaatatgtaggcggaggtcttaagtggttgttttaaaggaggtaacttggtatagttgatgtgaaaatcttgtaggtagttatgagatggaaccccagaacaaatgagagctagaaagatggataaaattcatggaagtgtagatttttagttaatcggaaataaattctcccagaatatagagatgggtttttatgttaactggttttgaattgaaactaaggacatgctaaggactaattacactgatgagaagaaagcatgtaggcttgagcctcagtcgcgtattctgacatcacagctgtcagggatgaggttatcactgcccgccgagtcactgtgggcagtaggaacttatagaagtctaaggatagtgagtggctgactgtccaggctatagctcaaggagcagacaagtacatttgacgaccttttataatcacagctagcgtgggaaaagctaatgttttcaaatgcatgcatatttgtgtcattgtatattctaggtatttccttaacttaataatttagatatttatccaaatattattgctatgggatttcctgcagaaagacttgaaggtgtatacaggaacaatattgatgatgtagtaaggtaagcattcttgattttctatttcttatattaataaattattttgatgtgttttatttagaaaagatcccgaaaacacagaccagtatttgcattttgatgtgttttggtaaaactctgaaagttttaacctaaagcacctgacagctctcactcggctggatgcgtcactggatgagaactggctagttatatagtcgtgtttgtttatgtcatgaagatttttttttttgtattccataatatgtctcttaccaggttattctctggcttgtattacagtacaaggttttgactttgtatttgggttaggccttgcttaagtaggtttgtttagttattcaccctgcggtatgagtgaccgatgtgttttatgtagcacttatacctgtagcagtgtttgatacaatgattttggagagacttgctggacattcattcaaaagagtaaatgaagagtatcataattttacaaaatttccaagtgtgattgttgcttagttcagaaaagtgtttctcaaggcccacttaaaaaatttagtttcagaataaaaatgcaattgtatgagtaaatgaacattaaatttttgttgcaaactatcatagtttttaacaattcattaatactgagtcttgctgtatttgctatgctggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngaaaatcaagaaaaaaaatgttgagcggtggtggtgcacgcctttaatcccagcacttgggaggcagaggcaggcggatttctgagttcaaggctggcctggtctacagagtgagttccaggacagccagggctacacagaaaaaccctgtcttggaaaacaaaaaaaaaaaaaaggaaataattaataataataataataaacaggaaagattacttctgtttttccattctaacatttttttgcccctttcctcattttgccttcctcttttccattttaaaaaaattagtgagttaacataatagtcactgttttaaactgtaatttgagtgacatttaatattttcacagtatagccagaggtaatgcagtcaccaccgataccaaattccagaacctttttatcactcctgaaagctcctggctgcccttcccttctgctggctgtctttcctgcacatggggagatttagtgtgttggacaataataatgttatgggcacaaactagctttgctatttaatagcggatactgttttctgtacatttatttatttactatttatatctgttaactattgtataacgagcttgcatataccatcttactaagtttgtagacggaatctaatccctatttcacttttataagtgccttaaaagaaattagaatcttcagttgccacagcttagaagtagcaagaagctggaagttgagtccgtgtctgtgagattccaaagttgattttttttcttttttgtttgttactgtatttaaggcaaggtctcactgtgtagctaaggccagtcttgaactcaaggtcctcctggttccatattctcagagtgctctgtgtataatcttatggtgatctggccagcaaaaattcaatttttaaaaagttttttaaccaggggagggatgcagtctggagtttgaactcagtcattactaggtacttcaccactgggcattacttaaagctgcttcccttaatatcttttttctgtacatcaaaggacaaaatctagaaacacttggacaaatcgagactatacttaaaaatcatgaagcctcgtgcaaaaccctgtatttctagcttatttttaaaagctgaaagagctgtcaaaactgaattcaacattcctgtgtgatggatcaagtatggttgtgtaatgctctctggcattaaattgttaccatttctccattaaggactgtttcttagtgaggtttccattgctggaaatgtgcctggcccaaggaatggcactattaggaggtgtggtcttgttgagggatgtgtgtgtgtcattttggggttaggctttgagaccctctcataactgcctgaggacagtctgctcctggtgtcctttggatgaagatatagaatcctggacaccaccatgcttcttgctgtgataactttcattatattgaaagttgttatattttaataatactgttaatgttttaccttgcctagtttattcaacttcattgataagtgtgtatatggaaaatgtacatacagtttgtcgctgtccaaagtttcaggcagccatatggaaggcgtgtgtatagtgtaaggtttcagctgaggatacattacattccttgttaaaccttagtgctgcagatgttctctcacttccacttgaagggctgtctatagcatttcttttaagttttcgtaacttttgtttctctgggaatatttcaatttatttctcattttttgaaggacagttttgctggatattttttatttctaggaccttaaatgttatattcttacttcctgttctgagggggaaaaatctgctgataccaacgctcctttgtgacatgttccttctctctgtatttgagactgtgggtctttctcaggagagggtcttgcataatacaggctagcctggagtccttgtatatatgcaaggatgtactaactgacttttgatcctcctgcttccacttgactagtgctctgatttcaggagtgcaccaccatcaaaggtttatgtagagatggggacagatttctcagagctctgtttgtgttatccaatgagatatgcccccagcctgtctttgtcttgtgatggtttgattgcgatctgtcttagtaagtctctgagtttttgtgaattgtggttcattgagccagaagttctttatattcttttatcaaatttgagaagttttgactgtacttctgtatataattatttttctggaactttaaagatccctaaggtagtccagttgctgttgtctcacaggttggttaatctctttacgtttcttcagtaagttgctttctgtattttgatatttatcctcctaccctagttcctgggtgtggaatgtggtttactccaggaaagaagattgactttctcactcttggtagccaatagctccttagccggggatgggactttggtcatcacttttctttgcttggcttgtgctttcacaggtcttgtgtatgctgttagggttgctgtgagttcatatgtgcatctggcctgttgtgtctagcaagcgctgtctccttgaagtcacctatcattcttgctcttacagccttcctgccctcttccacatagatgcctgagccttgaaaggaagggtatgatgcagaataccatttgctctgaacattttgaagtctttctttgcaggttgtatgtactaattgccatcaacagaagcttctctgatgagggttgagctgtgcactgtctgtggtttatttagcagtagtcattagcaatcattctattgctgtgtccacttacccgaggaatattggtaggttttccctaggctccatgcccatctagctacaggcttttggcctcattttgacaatgttagatgtggcttccatcttatagaacagacctaaatctaatcaaaaggtggttggttattcctataaacatttttattccacttgactgtacttttcagccatggctaggattacaagtatgaactactgtattgttctatttaaatttttattaagagttttttcatatatcttgatgatattctttcccattcttcaactcctcccaggtcttttcccacctcccattccgacaaatgtcatgttctttttttctttccttctcaagaagaaaaaaaagaaaatcaacaaaacccaataagacaaaaagtgacaaaacaaaacagaaaagcacaaaaaccatggagtccattctatgttggccaactactcctgtgcatgagcgctgattggagcgtagttgatatgttggagaaaactgatcttctgtttctcagtaggaatcaactgcaaatcgtttcttggttagaggcagggctttgtgtctgcttcagatttagtgctgagattttgtttggtttgacttgagcagatcttgcacatgctgaaacaatctgtgagtttgtgtgacacccttgttgtgtctggaagatgctgtttgcttagactcatttactacctctagctcttcccatctttcttccctttcctcggagtagatccctgaaccttgaggggaggggttcaataaatgcatcccatttagtactgagtgttccaaagcctctccatttgtacgctgtgttgatgtatatgcttaatttcatgtgggggttactgctttagatcattcagtttccagataaaaaacacaaacttttaaaaattatttataagccttaatgagcactaaagctgggctggtatctaccttctaggctattagtatctacttccttattggtagccctgagttataacttgccatatttcatctgggccactcttaactccaattggccagccttcatgaccgagttttcatgaatcacttaaccccactgtggcttctcctctctctattgtttcctgatcttctgcctcagaccccaagcctgggaacccaaaccccacctaactctcttcagcctagctataagctgtaggcatcttcattcaccaatcaaggatagctttcagggttatagagcattatttgatgtatgtgaggatcaccttggcccagaggtaaccagggccaatatttagcattacaatatataacaacagaccaaaccttaacggttttaaattaaggtgtaaggtttatacagcaaaggctggtaaatgtgaaattcacttgtaggtctaaatcttttagtacagaattcagcattgctatacatagcaacagaccaaacctcaacacactcttcagttgtgggtctctgtgttaatcaccatctactgcaaaaagaattttctctgatgagtgacacactcatctatagggagagcagtatgttaggaatatttctgtttctttaatagaataatagtagtagtaggttttcccctaggctcatgacttgtctagccttaaattcttagcctcactagcagtggcaggcatgggttctattttaaggaatgggtcttaaattcagtttttaaaaagtggttgtttgttcccataacatttatgccaatattggatcaatatatatgcccgcgagcatgcaggtctttgttgtgggtcacagagtttgtagctgggttatattgatgactacttttatcttccagtcgtgtgcaaaagtaccttccagcaccacgagtgctagtcagtagtgctgaatctctagttggctgtcagctcaatctctctgtgctcgatgacacaagtaagcagtatcttaagcaacaggactaccatctggttgtggaggaaaacagtagccttggcagtagccatgatgttgggattgcaagtatgtgctatcgcactttgttctttttttcaagacagggtttctctgtataccccttgcttcctggaactcactctgtagaccagaaatccacctgcctctgcctccgaagtgctaggattaaaggcgtgtggcaccactgcctggcctgtgctttgttctttatgtgggttctgggaccctaaacttagactaaggtgccttcctagtcctggaattttcctttttaaaatttttttatttgtttttgtatgttggggtgtgtgtgtgctatgccatgccacacttttagaggtcagaggacaacttataattctttccttttactgcatggttcagtttggtggcagttatcttttttttatcttctcagctacccatcttgttaataactcagaagctgcactttcctgcctcagccttccgaatgctggcggacaagtgtgtaccactacacctagctctttgtttctctttcactttattgatacttctgttcatcatttttcttgatcttacccgtgtcttttttttctttttttgagacagggtttctctgtgtagccctggctgtcctggaactcactctgtagatcaggctggccttgaactcagaaatccgcctgcttctgcctcccaagtactgggattaaaggcgtgcgccaccacgcctggcatacccctgtctttcattagctttctgagtatcattaagaccacacaaatctttgcctagtaaatttgctttctggtttttctgagagacagtttgttgactttttaacttattcgatttttgagtatccacacatcttattttggggtgtgagtatgcacacttgtgtatgcatgtatgtttttgtcttggtcttgtaaatgtgtgtgtatgtgtgtgtgtgtgtgtgtgtgtgtgtgtgcatgtggtgtgtgtgtgtgtgtgtgtgtgcatgtgcgcttgtgtgtgtgtgtgcatgtgtgtgtgtgtgcatgtgtgtgtgtgtgtgtatgtgtgtgtgtgtgtgtgtgcatgtgtgtgtgtgtgtatggggggaggtagctaaaaacaatctggatcttgtagggtcgaagatcctctctcttttcttgatggcccagctttcccttgttttctgtattgggtatctactatgctatacctatgcaaagattaccatgctaaactcatgcaaacttaagatctttggggctggagcagtggccgagtgtttggggacactggctgttctcacaactgcctgccaatctagtctgagggtacccgatagcctcttctaaactcgggtggcaggcactacatgctagtggcatgcaagtggtgcacagacatacattcaggcaaaatactaaatacacaaaatcataataaattaaagatcttttaggcttgggctttttttctaggtatagggaatgacttcctaaattttttttgtatgtgtaattaatctcagttgttattatctttaaatgttgggttctttgaaagatccaaaggaaggaaaaagaagtgggcagggcgagtagatttaaaatcccttgatgtcttcagttggtgggcgacagcttcctccatctacgtatgcgtgttcaaaagcagcaattagtgaccagcacacagatttaaaatattggaacgtacggcatttattattaactttggcttttgaaagttgtttgtaagtctctatagaggtatatcaatgactgtatgagaagtccttgttgtataagagctaaaatcagggctgtggagatggctcccttagtaaagttcttgctattctgagttcccattgttctttttttttttttaatagaagaaaaggtttattttactcacagttccatataacagttcattatcaaaagtaatgaggacaggaactgaaacagggcaggaacctggaggcaggagccaatgcagagagcatgaaggggcactcctgactggcttgctcagcatgctttcttttcttttctcttcttttcttttcttttcttttcttttcttttcttttcttttcttttaatattttttattattacgtattttcctcaattacatttagaatgctatctcaaaaatcccccataccctccccccaccaccacttccctacccacccattcccatttttttggccctggcgttcccctgtactggggcatataaagtttgcgtgtccaatgggcctctctttccagtgatggctgactaggccatcttttgatacatatgcagctagagtcaagagctccagggtactggttagttcataatgttgcacctacagggttgcagatccctttagctccttggatactttctctggctcctccattgggggccctgtgctccatccagtagctgactgtgagcatccacttctgtgtttgctaggccccggcctagtctcactgagttcccattcttagaactcatacaaaagccagcttgctcagcatgtttctgcgacctctgtgacaggaagcaggcagagaagactatcctgggggcttgctggccagttagcttagccaaaataactagctccgtgttcagtgagggaaccgttctcaaaaacagactagttgcaaagtcatagagaaatacctgttgttcccatgacacacacacacacacacacacacacacacacacctaaaattgtttaagttaaccttcattttctgtcagagctgactcactgaaagtgtcagcgtttgcctagattccctgggaaaggttccgcaagtgcagtcggtggtcagggctggcttctggggctgcttctctgtcctcttgaactactttggtttctttgtttctgttttgtggggttttttaagatttgtttttgttgttttgttgtggatttttggtaatactttctagcatttgaaatacatgtttatataaaataaatttaaaattcactattgtggcttatctagatttatttcctaagaaatctttcatgctcatacatcagcctcagtttatctcagtgagacagacacacagacacagcacagttggaaaggaggctcaacagggataggagggtgagagtggtgaggcgatgtgagacagacacacagacacaccacagttggaaaggaggctcaacagggataggagggtgagagtggtgagggcgatgtgagacagacacacagacacaccacagttggaaaggaggctcaacagggataggagggtgagagtggtgagggcgatgtgcagtcagttccttcaaggaagatgcagttctaggaggtgtcttaggtcgtgcagggttagggagcattgcctctcactgctgtctaatattttagcctctactatctaaatacatctctgtaggcaagtttgcccatttctctttggaatgtgctgtttcacttgtctttcctcacttgtctttctatgtgtcagactgagagaacagtgggggaagtgcggaatgtgtccctaagtaatcagttctctttgagacagttatccccccaccccttcaaatgatggaatgatgtactgtacccattaaagggctgctttcttctgttagacttgctgttgctcacatgctagctaagaaatcagaatgttcaactgttaaggggcacacagataggatttccctaagcctaaggtaaacacacggtaggaaagactcttgaaagaattatgagtttttagttgcaaatgacataaaatgtctttaccagaaaggaataatgctctggaggaagttcccattgtggaaagcagaagtttaggaaacgtggtgtaggggctacagtctgcttagacaccaatgcatggtcctacatcctggttgctgtctgtgaattcccaggtcttccagtgagatctttgaagaatctactgttctcttgtaccttgctgcccactctgtaggagtgagtgtctcacaacaagggaaagagaaaagaaatacctgcctctgatctcagtgtttgctaactggttgacataagggtggcacaatttccttatgaaatttttatacttcatccccctttcagaaatttgtagctgtgtttacatataagaagccgtggtctttgtttgtttgtttgggtgttcttggactttctagcttccaaagcttcggacagttaacttctgtggggcattgtgtgcatacgtggtgtttactttgtgttgactttcttttcaactgagttttcttttaaattgtttaaactgctttgattccttttgtagacacagctttataatgctttataagtcctttctttatgcctttataatatagcctttataaatcccttctgtgcccttagattcagataaatgttgactaaagaaattgatgggttatattttgctcagaataactgattgctaactctgctttattgttgtatataattactatattttctattgctagctcttaaataatcaagaagcagctttgcttaaattatcaagtagaaaagatttaacttatgaggaattgttaatatatctcctactactgactcggcatttttcttttggacagagaatagagaagtgaaaggtttagggctccctgcctttttcctgtttccagcattatacaccagtcaagcgtatggaattctagtttctttttgttctgttgctccactccaacctttagttgatactgtttttgtgttccttcttatacaccactttgtgctgttctgatttcatctctgagcactccttctgccattgtgatgaccgtgttttaaaatggagctttgtgagctctctgcagctaagtgttttttcctgaataatttgttcattacaaaagagaattctagagaatcctaccaagtccatagcattgttactgtgattgctgttttgagatggtgtccaactctaatcccagctgacttcaaactcagttctatagacctggctgtgtttacatgtgtgcggtggtaacatgcatggcacatgtcacttagtgggcttgacctttctttctctctctttctttctttctttctttctttctttctttctttctttctttctttctttctttctttctttctttctttctttgaatcatcaaagtatgacttcatgttttgtcttttaaaaaattacatttccctctgtgtttaaacaaatgagcctagtttatagttccccatggattacagttaaatcctctctgtagtcttcttttagattgggttgtagattcctaggctgctgctgaggcgaagcatttgcaatgctttacagtccagtatggtatctcactatgccagcatttccttccttgtctgatgtcagctctagaattacatgaacactttccctctgtttcctgacatttccagagttgtagtttccttctaaaaattatttataaaagagaactaaccaaccatttcaagatttttttttttaaagaaaaacctcagaagttaaaagaaccagattcctaatattttgctctatttttcttgtaattttataatgtattccgaggatgtgcccactttggtaacctgactgtgacacaaatgtattgtgtcatactgcttggttttctttctttaattgaaaataaaaaatagatattttttcatacaatattctgattatggtttctcctatcccaactcctcctagtttccctcccttctcccatacagatttacaccctttctgtctctcattagaaaacaggtgtctaaaaactaatagagtgaaataaagtaagcaaacaaactggaataggacaaaacaaacaaacaagaaaaacacaagacccacgtaggctcagagacacgtgtttgcacacatagaactcttataaaatcacaactggaaaccgtactatgtgtccaggagatctatgttctcggttttaatttacacgcacacacacacacacacacacacacaccctgctctgtaaatctcacagtgattgagcacatttggtgctcatcagtttctcgtactcctgggtcttcctgaccgacctaactctgacctaattgccttctgtgtgtgcagcctgaggtaccccttgcgatccttggggtcctcacttcttttacaggttgggctccctggttcccagaaccgattatgatttttcactctcaacatcttttacaactgagatagtgtatgggaaacaaatgacttgttgtagaacagtgcctttattgtattatatactcacccacgatttatagtctgtcttgtatagcattctaggctggaagtaaattttctgaaaaatcaaactttgtataattgtttttaggaagctagtgttaatggcagtgcgtttgtcgttttgtcttatgctgtctactttccatgccaactttagggtctggtgttctctttggcacttagaaataacgtagatatatatggctccatttgcggctcccctagaccccctttttaaagtcaattttattagctatttatgtcttcatcttgggaactcatgttggacctggagatgtaaactgacagaatgttttgctgaggctctaggtttaattgccagcactgcataaacccaggttggtgatacagacctgtagtcccagcaccccagaaatggagggaggagggtcaggaattcagggccagcccgggctacatgaaactattttctccttttgtctcattattaattcttcaccattataccttgctgagtcttctgtttcaggcctgaaggttaaacaaatttacatacataaagtacttaaataatacctggcatgtaataggtgctttggtacctgtgatcactgtgtggtttcacagctggttggaaggagtggcccctgctctgactcttcatttactagcttcacaccttggacaagcttcataatctcttgaggtttacttccttttcctgtaaaatgtaaattccatctctgcgatgttggtcagggacaagagaaagtatacatgtatacatgtgaaaaatgcttacagaactacattggtattgtacttttcagattgtggggttttttttttttccctgctaggaagattacattttaagcttttttttttttttcatggaagtctgtgagctgggtacacttgaactgctaatatcgttttgtcaagacgtgattgtaatttattagactgaagacatagatatgaaaacagtttttgataaagtcagctctacttcagaatgtataaatctgtgtaatgtaataactattaatgaatgaggggatatgtatttgtgttattaatagtatgtgagataagggtaaataaatctgttttagtcctgtgcagcattaatgtaatttgaaatattagctcatttttgttaatggtgttttttttgtttgttttgttttaaggtttttggattcaaagcataaaaaccattacaagatatacaatctgtaagtatgctttttttatttgtctctgttaaaataactaaataaaagttatttctttgttgaagataaaaatatatttagatatttttatatttgaggaactggattcctgaaaacagttgcagtctgatagagagagttgttgggtctcgaagcgtggtgatgaggtgcagcagcttggcacagcctccggttacttgatctgcttttacagacttggcacctcgcccatccttgagcccataatcatgtgataatttgaaatgtaatccacagcggagctgctgttagtattaacgatggcttctaaggagacagactccagggtggatggacagacttttgtttcctctgtgcttgttgatcaatatactgaaacagctatttgaatattttctgtgtataacctagtaagttatgcagcattgtttagttatctagtataggatttgagggattgctcattaaaacttattggcctatctttaaaccttcactttcttttgacttttggagtagtgacatgaaaacaggaaaggaagacaaatcattaaacaccctttgtctttcaaaaccatttttattttccccaaatactgagcatttttaaaaatttaaaagataaattaccatgtttctattatgtcctttaattttctatgtctatgatttatataacaggagaatgttatgcaatggtagaataccaattagtaattaaccattttctgtagactttatcaaatataactacaagtgttttctgttctgcttcgagtggctatttgaattgctacccagaaggatggagaattttctatgtcttgttatagtgctagatgttacttttattttttcagtctttaatgatatttctgttttgataagacttcaaagtattcatgtgcaatagttaccaatattatttctcttcgcttttgctgacttcagatcagaaaggtgcagccatggtgaaacatgcagatagagtgctcatatggctagttccagccctctagtagcctatagcttgatgtgaaagtaggagggagcaggagagaagtgtggacaaagtaactggccccacaggaggcctctgtaaaagaccagatgtgtgggctgtgattaacttctgataccttctttcttctatccctgcttgttatatacttgtaagactaagaggagtttctgttttatttcttttaattttaagattatttctttgcaaacataaatttaaagatcttgaaatatttccatggcttttctactaatgaaaatcaataggagttatctattagacctgggaggatgagccaaggcaagtcagaagattgatagtataatggtatttgaaatatggcagataactcattttgggcaggtggtggtgtatgctggcttaggtggggttgtgctaataaaaggtggataggagaaccacaagactgtttctgaacagctgcattcagaaggtgactgaaaaaggacaagaactgttgaaagctggaattgatgaaatgaatcatttcagactcacactgtcaggtttggggatttagagaggtcccaatagggaagtagaaagacattagaagacacatttctgcctgagctgaaatcttatgcctgtttaacatatctaaagcacagggaggaaattcttttcattcctgcctatagtgacttcctgcccctagaatttagggattaggtttatgctgtctcctttgttgtatttcagtatagttagaggtggcattgggtggacctaggaacttgatttgagtttccaagcatttgattcccaatttaatgaaccatctctttattagttgagagcagcctttagtgcatatgaacttattcccttgtcatttggaactgaggctttcagaatggcaaaggatctgaagggtccttttagcagtgccttcttatcttatagacagggcattaggcctaggaagttaaatgaggtagccaaagacaggtaggtgcataataacagactacccactgtttgcaccagaatcccttttgtttgctggttagctcttcgttttatttacttcaaaagtttttaaacatatacaaaattgagtgttttaatttgagtaccccctctcccctgcctactgtgtatctgattttaggcaagtgagactagccacaacagatgtttttgttttatttctttttgtcttaggataggaattacaggtagtatgtattttttttttcttggaaatgtagatgtttgaaggtcctaaagtatttttcactggacatctgtatagttagtagtttgtgagaccttttatagcagcagtgttgcacatgaatgaagaactatcagcctaagctttctgataatctagcttatctattattattaatcagttattttgaaaaagggcaacattaattaatcagttttatatgagtgtttttaaaatttctttgttgctccctgttcagagaatacaagattttaagtttttattatattttagtgaatatttgctgtacttggcaaacatttaactgtgttatttttctgttaagatttcctttgtaaaacactgtagagtgaagaagagagctccctaccatgtagttctatggcaaggcctagttgtctgcaagtttgcctttctggtttcactcctcctcttaatttctgttgccaccttgggaaacctcattttccttgttttttttttttttcatttctcttttcatataagccaatttaagataaggacaaaaatatcgtttgagttttaattacaaagaaaaatttaaatccaaattgttatttgctatcttctattttagtatgtggagtgacttactgctaatatgccataagaaatttaaaagaaactccgctgtgaattttggctatataccagagattctaactaaggtggaaggtttcttcttgaccctgtgaccctttctttctcttgagcactgtttcacaggcagccctagcatgtcctcccaaagcccctccgcttgcctataaggagctgcatgctcccctccccccccaagtcaattgttaggtctgtcttcagtgacaaatactgctcatgtttgtgctgtaaaatttgtcactgctttttcatttaagacttgaatgtttctgttatgttgaatgaaactgtaatagaagttgttggatttagttgagcaaggatactaagcttgagttcctgtctcacggtgacttcatgttgttattaggaaagcttttaagggcctttctaaatcttagcttttccatatatacatatgcctcacatatacaatggggatgtaaactgttacatgattgtgagggtgaaaacatggatgtcagctgtaaggtgcccatatcctgtagacttcagttgttactgtgttcctttcaccttaactgatgatacatgacaaccagtttgtaatggtgatcttaagcagtgcttattaaaccaaacttttcagagtgtttgttccatctttctctggggtgggaccctcccttcccctcctctccccttccctgcatcacctccgcaggcaattgggatccctgaccctagaccagaaagtgtggcaaactgaaaaatctgacttgtaggacactaacaaccggcttcttagggtatgtgcctagcttcctcttgtttcctgattgtatccttaattcttgactgtcttccactgtgggctcttcaccacacagcacctctcagaagagcagaacctggcttccctgtgtggagttctaacacttggaggtggagggagaagggaattcagagccagtcttgggtatatgagatcctgactcaaggaaaaccaaagaggaagggaggaaagagaatatagaatatgtgatcttttgtatatgtgtcagttttcttcttcctatctcatttttaggtaagcagacatttagcagagtatttagcaaggatgcatacgtcatctaataaattttctcttttcaaaaacagtacatcaggtaatacactaaaagaaaaacacatgtgtgtgtccgtgtctgtgtgtgtgtgtgtgtgtgtgtgtgtgtgtgaatacagaagttaattcccctcaggtctgctccattgggctgtagtttatggataatttgttcaatctttgtgtgaactgggttttgaaatacagttgagttgtacaaattccagatgcccagtgcaggcccacagctatttatttggaagtcttggatcagttttattttggtacatagaaaatttcagttttcaaaaaactaaaaaactaaataaaacaagaaaatccatatcttttgtgttactctagtatccactgtggtagactagtcggtactcagcaggtatgttggttgaacaacctcagattgggtcctgttcgagttgagattacctatttataactttggagtttgagatttgggctaaggaataatggaactttgttttaaaacactaacttttatttttcagtaatttctttttgtttgtttgtttgttttttttgagacagggtcttgtatcccaggctggcctaggactcactagatagcaaaggctaatcttaaagatataatctttcccagtaactcttctgaagtgctaggattacagcctgtggtaacactcctagcttatttgaataatgcttaagtgtctgatttccttagtagttggagtcaccaggatgcttctgaccccactaatatgtaggatacccttcatagtatcactgattagtgttattattgaaaagctaagtgtttgtcttaatgtgtcagtattttactatcagtgggttttagttattttattgtgatctggtattaaattttgtactctgagagattattggaaatgagatttgtatataaaagagtaaaggtctggcttacaatttttagtaagcattgtgttaataattaaattagtatcattcagttgtcttttacatttcctttgttctttttctttatttttaacatgtatgttttaagtaatggtttaagattgtatgtgatcatctgtcaggtaaagataatagtaagagtagctatttattcataggtatttgtgaaataaaaaatacattctaaagccatgtatagtctttatccaagaaattacagggtcagtgcagttgaatttacagtgttgcatgttgatgtcacaaattctgtgaacaaatatatgcacacaaattgcatgcatgcgtttaacttttattaaagctttggtctccttaattataagaatgataatagtacctacttcagaattcttgaagttaacggaaatagtgactgtaaaaacacttagcgcagtgtttttacatgatagaaaaggtggtatgatagaaagggtggataaatattgctaatattgatactcttccttccagtgtgaaaggtaactttatgccacatttaaactttcttgtagatgtgctgagagacattatgacaccgccaaatttaactgcagaggtatgtataaacataaccacagcatactgtataactaaagaccaatagacttgtcttttactgcctggtgataattatcaagattagtgagataaaaatcttaagaatggcctttgacaattaaaaaaagtgtatttaatgttagagttgttctttaagacctatctattgtcaggaaaactaaatcacagaatacttggagaggtcccaagactaaactaggattggaggtgcttattgacggtgtgggacagctagcgctgctggaaacaatcacaagaagagagcagaaccattttaacttttctacatcgaagaatggcataaagttaggaaaagatgtagcattggtctgtctgtctgtctgtctgcctgtctgtcttctcagaatcatgaagcactaaggagtaagtaagaacagtttctggggaccgacagacctaggctactgctcattaggaaacatgccatggttgaaggtcacttagctttaaatgtacattttaacagactcttgaatgttcttgtgtgccactggggaaatgaggtcgggagcacagttagacagatggttaagtaaaagctggcctgcagcctcttggtgaatgtagtttgccattgtttaccacagagctttcctgtcatggaaaggagtaaatggatggattgttcttgtaccattttacgatggcttgctttaggataagtcagagtttttacatattagataatatggcagataatcagaacagtaatatcaccaggattttttgttttaattttaagacaagggtctcagggtctcagtgtcccagagtgaccctgaactcaatgtttagctgagggtgactttgaacttgtgatcccaattctcctgcttttactcctcaagtattaggattacagacttgcaccacatcctcagttgtgtgtttactcaaggcagggatgagcccagagctgagcatcctaagcaagcactctgcgaactgagctacatcccagagttcataccaggatttaaggatctcaataggatagaatcaaaacagatactagtaagataaaaaccagtagtgatagaacggaagtcttgcttctagataatagcatcttgccttcaaaaacttaactctgactatagagaacaaagacatcttagattcttaattcatgtgaaaaaaatctgaaacttaatttgctataaactttacttcagttgtatgtttttctgtgagtgattaatctcatgtatatggaaatataatgtttgtgagaccattttaaaaacaagtcactgggtaattttattatgggataggaaaagtcagtcttttccatagttgactctattagtaattatactttcttcggagcatgtctggcaatgctgtagtaatatctgctattggtcctgatagaagttactacttgacaagaggcctgggtgacgtgcatttggattcagttgtactgataggctatgacgtgttcccttcatgcacagattcatcctccctggagtgaagagcacaatgcttgtttccatgtctaatgaatgcatttaagaattaataaaagactttctttaaaatctaggtttaattagtaataaattaaaatttcctgaaagttaggcttcttttaagaaccagtaagtttatatataacattttgaaagttaacctatgtttttaaataaaaaatttaaaattttcttacactgggattatctttttgcaacagttgcacagtatccttttgaagaccataacccaccacagctagaacttatcaaacccttctgtgaagatcttgaccaatggctaagtgaagatgacaatcatgttgcagcaattcactgtaaagctggaaagggacggactggtgtaatgatttgtgcatatttattgcatcggggcaaatttttaaaggcacaagaggccctagatttttatggggaagtaaggaccagagacaaaaaggtaagctgtttactttttccttcctccctctttgtggaccaagaatttattgggaaacaggttttctccctcttgctttattgaggtataaccaacaaagtcttaatctacttacagtgtgatgctttgagaactgttatattgtggttgtatccacttagtgtatccctcatccctggtatccccaccctcttccttagctgtactgagaacatccaagacctacctggagtaggtgctaggcacacagtatggattttgatgacaacttgaatgccattacctagtaaagcaaggtatttaatttgatggtaaataaaacattttctgatgggggtattcactagtatagttaactaatcaaagattcattggttattcagaaaactaaagactgttgaattagtggcatgttttgtctatggtacaattgaaaacaaaagcaaattcttggactgctttttcagaggactcgtttagttagtgtaacaccaagattctttgcatgtttttctttctccaagcacagcacctatagtacttcagatgaattgaaagctcagggtagcagtgaaagtgccccaacataaggtcataaactcacttaacctttgagttggtttgcagtcttttttgtagacattgtaagtgacaacatcagtttgcaatgccaagggttggacatggctgctctggggagtaagacatttgaaacttgattctagtattaaatttggacttgtgccccacccccgcttctcttctgcctcctctcccttctgtctttctcctcctctactccattcttcccccttctcctttttttgagccctgattttatctggatcaactttgggccatgcccatcacactaaggtctgtggctgcagcggtcctgggccctgtacttctctttcacctgctttttaaaaaccctgtcgttataactcttttgagtttgtacaagaatatcaagactgtttgttcattggtgggagttcacaaaattacatctttaatgcagtaaaaaagtcatgtgttagaaaatcagatttaagctagagactcctcaactctgactcccgatgaagtgttcagatgttctgttattcgatgtatgtggtatatacataaccataaattgttgttggtagcttccatttgccttcagacaaaatataaaggaacttctaacaaattatgtctcatttctcccatttaaaaaatcagtaccccttacctgagaacagtaggtatctaaatgggttgattctgttcaatagtgaaatttatgataaacaagttttaaaaacaagttgaaagcttgccattgtttgactcttacatcatccttgctctcagtgttatttttattcttgtttagtgaaaataaattatgaaaactcttatttcacctatgagagaaatatggaacataatatgtttttgaccaattaaagtaggctgtgtcagataaaatctctaagactagatacgatcatctattagtttctttgccttcaagatcattatctctgtggggcaggaaaagattatggaccattttaattttcaggttaaagcattaaactgcttgacagcacagcgttgtctggcttctagatatcagtggacctgt

SEQ ID NO: 99 - DNA, 3160 bp, M. musculus
cctcccctcgcccggcgcggtcccgtccgcctctcgctcgcctcccgcctcccctcggtcttccgaggcgcccgggctcccggcgcggcggcggagggggcgggcaggccggcgggcggtgatgtggcaggactctttatgcgctgcggcaggatacgcgctcggcgctgggacgcgactgcgctcagttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggcggcacatccagggacccgggccggttttaaacctcccgtccgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaaaggttttgctaccattctaatgcatgtattcgggttagggcaatggaggggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaaaaaaaaaa

SEQ ID NO: 100 - DNA, 20 bp, Artificial Sequence
  antisense Oligonucleotide
aggggagagagcaactctcc

SEQ ID NO: 101 - DNA, 20 bp, Artificial Sequence
  antisense Oligonucleotide
atcaatattgttcctgtata

SEQ ID NO: 102 - DNA, 20 bp, Artificial Sequence
  antisense Oligonucleotide
cttgtaatggtttttatgct

SEQ ID NO: 103 - DNA, 20 bp, Artificial Sequence
  antisense Oligonucleotide
aatttggcggtgtcataatg

SEQ ID NO: 104 - DNA, 20 bp, Artificial Sequence
  antisense Oligonucleotide
tggtccttacttccccataa

SEQ ID NO: 105 - DNA, 20 bp, Artificial Sequence
  antisense Oligonucleotide
ccactgaacattggaatagt

SEQ ID NO: 106 - DNA, 20 bp, Artificial Sequence
  antisense Oligonucleotide
tcttgttctgtttgtggaag

SEQ ID NO: 107 - DNA, 20 bp, Artificial Sequence
  antisense Oligonucleotide
gagagaagtatcggttggcc

SEQ ID NO: 108 - DNA, 20 bp, Artificial Sequence
  antisense Oligonucleotide
aggacagcagccaatctctc

SEQ ID NO: 109 - DNA, 20 bp, Artificial Sequence
  antisense Oligonucleotide
ctgctagcctctggatttga

SEQ ID NO: 110 - DNA, 20 bp, Artificial Sequence
  antisense Oligonucleotide
tagtgcggacctacccacga

SEQ ID NO: 111 - DNA, 20 bp, Artificial Sequence
  antisense Oligonucleotide
ttctacctcgcgcgatttac

SEQ ID NO: 112 - DNA, 1579 bp, M. musculus
tgccctgcatggtgtctttgcctcggctgtgcgcgctatggggctgcttgttgacagcggtccatctagggcagtgtgttacgtgcagtgacaaacagtacctccacgatggccagtgctgtgatttgtgccagccaggaagccgactgacaagccactgcacagctcttgagaagacccaatgccacccatgtgactcaggcgaattctcagcccagtggaacagggagattcgctgtcaccagcacagacactgtgaacccaatcaagggcttcgggttaagaaggagggcaccgcagaatcagacactgtctgtacctgtaaggaaggacaacactgcaccagcaaggattgcgaggcatgtgctcagcacacgccctgtatccctggctttggagttatggagatggccactgagaccactgataccgtctgtcatccctgcccagtcggcttcttctccaatcagtcatcacttttcgaaaagtgttatccctggacaagctgtgaggataagaacttggaggtcctacagaaaggaacgagtcagactaatgtcatctgtggtttaaagtcccggatgcgagccctgctggtcattcctgtcgtgatgggcatcctcatcaccattttcggggtgtttctctatatcaaaaaggtggtcaagaaaccaaaggataatgagatgttaccccctgcggctcgacggcaagatccccaggagatggaagattatcccggtcataacaccgctgctccagtgcaggagacactgcacgggtgtcagcctgtcacacaggaggatggtaaagagagtcgcatctcagtgcaggagcggcaggtgacagacagcatagccttgaggcccctggtctgaaccctggaactgctttggaggcgatggctgcttgctgacctttgaagtttgagatgagccaagacagagcccagtgcagctaactctcatgcctgccccctgtcatttctcaacttgctttttaaggatggagggaaagctcgggcatcgggaggtccacagtgatatctaccaagtgcagcagtgcaggacccagagttgtcttgctgcggcgttcactgtaaggagtcgtggctacaggagtccgtggcccgcagcttgtgctcgtagagggcacctggttgccatcagcagggtactggctaaataaatctgtaattatttatacaatggcatctcagaaactctagcaggtggggcagaaaacaggtagtggaatgatgggtagagaaacagcttttaaaacacattccaaggcaggtaagatggcttttgtgggtaaaggagcttgctgcccaaacccggttacctgattttgatccctgggacttcatggtaaaagggagagaaccaaatccagagggttgtcatttgacctccatgtgtgctctgtggtaatgtaccccgtgtgtgcacatgtgcacatatcctaaaatggatgtggtggtgtattgtagaaattatttaatccgccctgggtttctacctgtgtgttaccatttagttcttgaataaagacacactcaacctttatatttaca

SEQ ID NO: 113 - DNA, 2000 bp, M. musculus
gtccccccttgtccttccaagctgttcgcaccacagcctttcagtccctgctcgccgcccgtgtgccccgggaccctgaccttcgcacccctggacccattggctcctttctccttccatcccgccgaactccgactctcgagccgccgttgtctctgggacatggtcctctgcgtacagggatcttgtcctttgctggctgtggagcaaattgggcggcggcctctgtgggcccagtccctggagctgcccgggccagccatgcagcccttacccactggggcattcccagaggaagtgacagaggagacccctgtccaggcagagaatgaaccgaaggtgctagaccctgagggggatctgctgtgcatagccaagacgttctcctaccttcgggaatctgggtggtactggggttctattacagccagcgaggcccggcagcacctacagaagatgccggagggtacattcctagttcgagacagcacccaccccagctacctgttcacactgtcagtcaaaaccacccgtggccccaccaacgtgcggatcgagtacgccgattctagcttccggctggactctaactgcttgtcaagacctcgaatcctggccttcccagatgtggtcagccttgtgcagcactatgtggcctcctgtgcagctgacacccggagcgacagcccggatcctgctcccaccccagccctgcctatgtctaagcaagatgcacctagtgactcggtgctgcctatccccgtggctactgcagtgcacctgaaactggtgcagccctttgtgcgcaggagcagtgcccgcagcttacaacatctgtgtcggctagtcatcaaccgtctggtggccgacgtggactgcttacccctgccccggcgtatggccgactacctccgacagtaccccttccaactctgactgagccaggcaccctgctctgcctcacacagtcacatcctggagggaacacagtccccagctggacttggggttctgctgtcctttcttcagtcatcctggtgcctgcatgcatgtgacagctggaccagagaatgccagcaagaacaaggcaggtggaggagggattgtcacacaactctgaggtcaacgcctctaggtacaatatggctctttgtggtgagccatgtatcagagcgagacaggcaggacctcgtctctccacagaggctggacctaggtctccactcacttgcctgcccttgccacctgaactgtgtctattctcccagccctggtttctcagtctgctgagtagggcaggccccctacccatgtatagaatagcgagcctgtttctgggagaatatcagccagaggttgatcatgccaaggccccttatggggacgcagactgggctaggggactacacagttatacagtatttatttatttattctccttgcaggggttgggggtggaatgatggcgtgagccatcccacttctctgccctgtgctctgggtggtccagagacccccaggtctggttcttccctgtggagacccccatcccaaaacattgttgggcccaaagtagtctcgaatgtcctgggcccatccacctgcgtatggatgtgcccacttttttctcccaagcctcttttgggaggctgggtggccagacagacaggagccagaaacacaagggctcccactcttctcctcacagggcagcaccatggcttcatagagctggcttctctatgttgtgccccacctcacccccctgccgaggggcgtgtgctgggtcgggaagtggatgcttatccaagggccgcagatgtagctcccttgtgtccgtttcctgcctaggaagttgcctgcacgtgagagagggagaaatacatacacacctaacaagactttagaaaacaagtgttagaacacaagaaccagtttgggagtttttcttccactgatttttttctgtaatgataataaaattatgccttccacttatg

SEQ ID NO: 114 - DNA, 15 bp, Artificial Sequence
  antisense Oligonucleotide
cacagatgacattag

SEQ ID NO: 115 - DNA, 19 bp, Artificial Sequence
  antisense Oligonucleotide
ttccatcccgccgaactcc